(19) United States
(12) Patent Application Publication
(10) Pub. No.: US 2025/0367180 A1
(43) Pub. Date: Dec. 4, 2025

(54) STABLE OPHTHALMIC FORMULATIONS OF A FLUORINATED INTEGRIN ANTAGONIST

(71) Applicant: OcuTerra Therapeutics, Inc., Boston, MA (US)

(72) Inventors: D. Scott Edwards, Bedford, MA (US); Ben C. Askew, Marshfield, MA (US)

(21) Appl. No.: 18/876,377

(22) PCT Filed: Jun. 27, 2023
(86) PCT No.: PCT/US2023/026304

§ 371 Date: Dec. 18, 2024

Related U.S. Application Data
(60) Provisional application No. 63/355,813, filed on Jun. 27, 2022.

(51) Int. Cl. A61K 31/444; A61K 9/00; A61K 9/08; A61K 47/02; A61K 47/18; A61K 47/40; C07D 471/04

(52) U.S. Cl. CPC A61K 31/444; A61K 9/0048; A61K 9/08; A61K 47/02; A61K 47/183; A61K 47/186; A61K 47/40; C07D 471/04

(57) ABSTRACT

The invention provides stable ophthalmic formulations, a unit dose containing such formulations, medical kits, and methods for making and using such formulations and unit doses to treat patients suffering from a disorder mediated by an αv integrin, such as diabetic retinopathy.

### CROSS-REFERENCE TO RELATED APPLICATIONS

This application claims the benefit of and priority to U.S. Provisional Patent Application Ser. No. 63/355,813, filed Jun. 27, 2022, the contents of which are hereby incorporated by reference in their entirety.

### FIELD OF THE INVENTION

The invention provides stable ophthalmic formulations, a unit dose containing such formulations, medical kits, and methods for making and using such formulations and unit doses to treat patients suffering from a disorder mediated by an αv integrin, such as diabetic retinopathy.

### BACKGROUND

Disorders mediated by αv integrins impact a significant number of patients. For example, age-related macular degeneration (AMD) is the leading cause of blindness in people over the age of 55, and diabetic retinopathy (DR) is the leading cause of blindness in people under age 55. Both diseases are characterized by new blood vessel growth—choroidal neovascularization in AMD and retinal neovascularization in DR. Macular edema occurs when fluid and protein deposits collect on or under the macula of the eye (a yellow central area of the retina) and cause it to thicken and swell (edema). Diabetic macular edema (DME) is similarly caused by leaking macular capillaries. DME is the most common cause of visual loss in both proliferative and non-proliferative DR. Thrombosis of central retinal vein (CRV) and its branches is the second most prevalent vascular pathology after DR, and results in abrupt decrease in visual acuity and is accompanied by macular edema. Thus, anti-angiogenesis treatments are useful in combating these conditions.

Proteins that are αv integrins have been shown to be involved in ocular angiogenesis. Expression of αv integrins is upregulated in various diseases or conditions, such as AMD and DR, and in mouse model of oxygen-induced retinopathy (OIR) or retinopathy of prematurity (ROP) model. Also, αvβ3 is expressed in new vessels after photocoagulation, but not in normal choroidal vessels, in the laser-induced choroidal neovascularization model for AMD. Administration of αv integrins antagonists, such as a cyclic RGD peptide, have been shown to inhibit retinal and choroidal neovascularization.

Certain compounds that inhibit integrin are described in international patent application publication WO 2014/124302. Additional formulations suitable for ophthalmic use would benefit patients.

The present invention addresses this need for additional formulations and provides other related advantages.

### SUMMARY

The invention provides stable ophthalmic formulations, a unit dose containing such formulations, medical kits, and methods for making and using such formulations and unit doses to treat patients suffering from a disorder mediated by an αv integrin, such as diabetic retinopathy. One benefit of the formulations is achieving good stability during storage while also being suitable for topical administration to the eye of a patient. For example, aqueous ophthalmic formulations containing a cyclodextrin and a compound of Formula I desirably contain at least a 1.25:1 mole ratio of cyclodextrin to compound of Formula I in order to minimize the formation of the solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate upon storage of the aqueous ophthalmic formulation. Formula I has the structure:

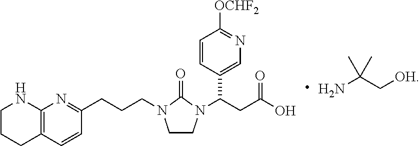

Experimental results described herein show formation of the solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate upon storage of a aqueous ophthalmic formulation containing a 1.1:1 mole ratio of hydroxypropyl-β-cyclodextrin to compound of Formula I. By contrast, no solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate was observed in aqueous ophthalmic formulations containing a 1.25:1 mole ratio of hydroxypropyl-β-cyclodextrin to compound of Formula I. These features and benefits, and other features and benefits of the ophthalmic formulations are described in more detail below.

Accordingly, one aspect of the invention provides an aqueous, ophthalmic solution, comprising:
a. from 4.7% (w/v) to 5.3% (w/v) of a compound of Formula I:

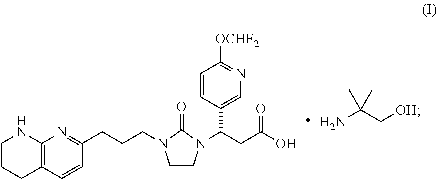

b. from 14% (w/v) to 18% (w/v) of a cyclodextrin;
c. from 0.1% (w/v) to 5% (w/v) of a buffer;
d. from 0.01% (w/v) to 1% (w/v) of a preservative; and
e. at least 75% (w/v) water;
wherein the solution has a pH in the range of 7.5 to 8.7.
   In certain embodiments, the cyclodextrin is 2-hydroxypropyl-β-cyclodextrin. In certain embodiments, the buffer comprises boric acid.

Another aspect of the invention provides an aqueous, ophthalmic solution, comprising:
a. from 4.9% (w/v) to 5.1% (w/v) of a compound of Formula I:

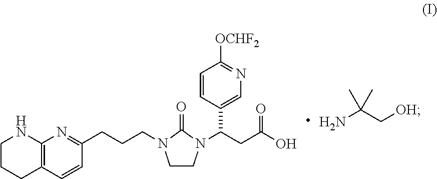

b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.2% (w/v) of a benzalkonium salt; and
e. at least 75% (w/v) water;
wherein the solution has a pH in the range of 7.8 to 8.5.

Another aspect of the invention provides an aqueous, ophthalmic solution, comprising:
a. 5.5% (w/v) of a compound of Formula I:

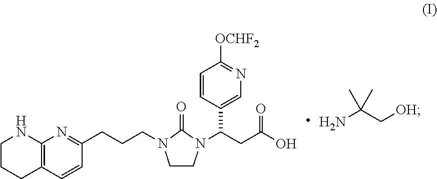

b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water;
wherein the solution has a pH in the range of 7.8 to 8.5.

Another aspect of the invention provides an aqueous, ophthalmic solution, comprising:
a. 5.5% (w/v) of a compound of Formula I:

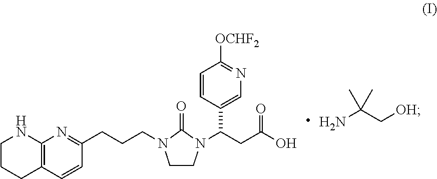

b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water;
wherein the solution has a pH in the range of 7.8 to 8.5.

Another aspect of the invention provides an aqueous, ophthalmic solution, consisting of:
a. from 4.9% (w/v) to 5.1% (w/v) of a compound of Formula I:

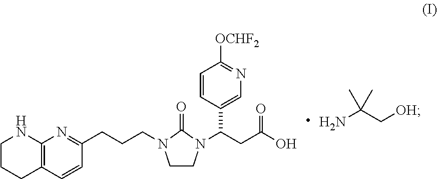

b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.5% (w/v) of a benzalkonium salt;
e. at least 75% (w/v) water; and
f. one or more excipients independently selected from the group consisting of a pH adjuster and a tonicity modifier;
wherein the solution has a pH in the range of 7.8 to 8.5.

Another aspect of the invention provides an aqueous, ophthalmic solution, consisting of:
a. 5.5% (w/v) of a compound of Formula I:

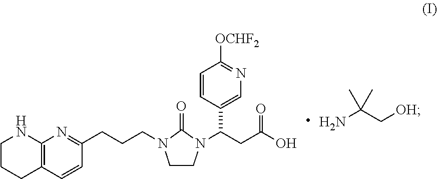

b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

Another aspect of the invention provides an aqueous, ophthalmic solution, consisting of:
a. 5.5% (w/v) of a compound of Formula I:

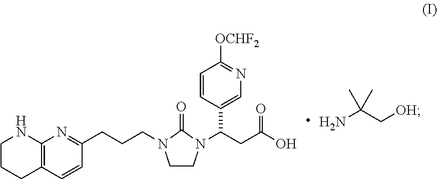

b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

Another aspect of the invention provides an aqueous, ophthalmic solution, comprising:
a. from about 4% (w/v) to about 6% (w/v) of a compound of Formula II or a pharmaceutically acceptable salt thereof:

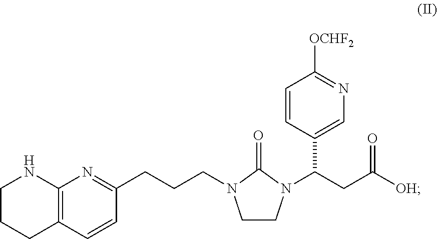 [SMILES: O=C(O)C[C@@H](c1ccc(OC(F)F)nc1)N1CCN(CCCc2ccc3c(n2)NCCC3)C1=O]

b. from 14% (w/v) to 18% (w/v) of a cyclodextrin;
c. a buffer;
d. a preservative; and
e. at least 75% (w/v) water;
wherein the solution has a pH in the range of 7.5 to 8.7.

Another aspect of the invention provides a method of treating a disorder mediated by an αv integrin. The method comprises topically administering to an eye of a subject in need thereof a therapeutically effective amount of a solution described herein to treat the disorder. In certain embodiments, the disorder is macular degeneration, diabetic retinopathy, macular edema, diabetic macular edema, or macular edema following retinal vein occlusion. In certain embodiments, the disorder is diabetic retinopathy.

Another aspect of the invention provides the compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid hydrate. In certain embodiments, the compound is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

Another aspect of the invention provides methods for preparing ophthalmic formulations. For example, in one aspect, the invention provides a method of preparing an aqueous, ophthalmic solution, wherein the method comprises:
a. providing a first mixture comprising water, a cyclodextrin, and a compound of Formula I:

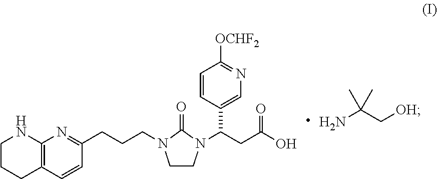

b. admixing a preservative and the first mixture, to thereby provide the aqueous, ophthalmic solution.

### BRIEF DESCRIPTION OF THE DRAWINGS

FIG. 1 depicts a ¹H NMR spectrum (400 MHz, in deuterated DMSO) of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate, as further described in Example 5.

### DETAILED DESCRIPTION

The invention provides stable ophthalmic formulations, a unit dose containing such formulations, medical kits, and methods for making and using such formulations and unit doses to treat patients suffering from a disorder mediated by an αv integrin, such as diabetic retinopathy. One benefit of the formulations is achieving good stability during storage while also being suitable for topical administration to the eye of a patient. For example, aqueous ophthalmic formulations containing a cyclodextrin and a compound of Formula I desirably contain at least a 1.25:1 mole ratio of cyclodextrin to compound of Formula I in order to minimize the formation of the solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate upon storage of the aqueous ophthalmic formulation. Formula I has the structure:

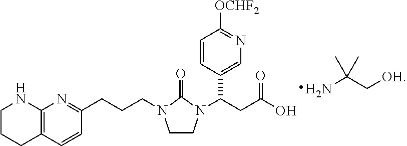

Experimental results described herein show formation of the solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate upon storage of a aqueous ophthalmic formulation containing a 1.1:1 mole ratio of hydroxypropyl-β-cyclodextrin to compound of Formula I. By contrast, no solid precipitate compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate was observed in aqueous ophthalmic formulations containing a 1.25:1 mole ratio of hydroxypropyl-β-cyclodextrin to compound of Formula I. These features and benefits, and other features and benefits of the ophthalmic formulations are described in more detail below. The formulations contain an αv integrin antagonist and, therefore, are useful for treating disorders mediated by an αv integrin.

A wide variety of disorders can be treated by inhibiting processes mediated by αv integrins. Compounds that are αv integrin antagonists represent a useful class of drugs for treating those disorders. Integrins are heterodimeric transmembrane proteins through which cells attach and communicate with extracellular matrices and other cells. The αv integrins are key receptors involved in mediating cell migration and angiogenesis. Antagonists of the integrins αvβ3 and αvβ5 are useful for treating and preventing, for example, bone resorption, osteoporosis, vascular restenosis, diabetic retinopathy, macular degeneration, angiogenesis, atherosclerosis, inflammation, viral disease, tumor growth, and metastasis.

Proteins that are αv integrins have also been found to be involved in ocular angiogenesis, a process that can lead to various ocular diseases, such as age-related macular degeneration (AMD), diabetic retinopathy (DR), diabetic macular edema (DME), and macular edema following retinal vein occlusion (RVO). Pro-angiogenic growth factors, including VEGF and FGF, are up-regulated in AMD and DR, which, in turn, stimulate αv integrin expression. In the well-established mouse model of oxygen-induced retinopathy (OIR) or retinopathy of prematurity (ROP) model, αv integrins and the ligand osteopontin are overexpressed in neovascular endothelial cells during the peak time of retinal vessel growth. Cyclic peptides mimicking the arginine-glycine-asparagine (RGD) binding motif, through which αv integrins bind to their extracellular matrix ligands, have been shown to inhibit retinal neo-vascularization in the mouse OIR model via various routes of administration (e.g., subcutaneous, intraperitoneal, periocular, or topical). Also, in the laser-induced choroidal neovascularization model (rats), a well-accepted model for AMD, integrins αvβ3 and von Willebrand factor are expressed on endothelial cells of new vessels after photocoagulation, but not in normal choroidal vessels. In this model, intravitreal injection of a cyclic RGD peptide significantly reduces the development of choroidal neovascularization. In humans, expression of αvβ3 and αvβ5, which are not expressed in normal retinal tissue, is observed in vascular cells in the eyes of DR patients, and high levels of αvβ5 expression is primarily observed in ocular tissues in AMD patients.

Disorders of the retina (which is located at the back of the eye), including macular degeneration, DR, DME, and macular edema following RVO, are difficult to treat by systemic administration (e.g., oral, intravenous, intra-nasally, or inhalation) because the retina is difficult to access from systemic circulation due to the blood-retinal barrier. Therapies that topically administer a formulation to the eye would be beneficial to patients.

### I. DEFINITIONS

To facilitate an understanding of the present invention, a number of terms and phrases are defined below.

The terms “a” and “an” as used herein mean “one or more” and include the plural unless the context is inappropriate.

The term “about” means within 10% of the stated value. In certain embodiments, the value may be within 8%, 6%, 4%, 2%, or 1% of the stated value.

An “αv integrin antagonist” refers to a compound which binds to and inhibits or interferes with the function of either αvβ3 or αvβ5, or a compound which binds to and inhibits or interferes with the function of both αvβ3 and αvβ5 (i.e., a dual αvβ3/αvβ5 antagonist). The compounds bind to the receptors as antagonists, blocking or interfering with the binding of the native agonist, such as vitronectin, while not provoking a biological response themselves.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Specific stereoisomers can also be obtained selectively using stereomerically pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁻, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. OPHTHALMIC FORMULATIONS

One aspect of the invention provides ophthalmic formulations. Exemplary ophthalmic formulations are described in more detail below. One benefit of the formulations is achieving good stability during storage while also being suitable for topical administration to the eye of a patient.

### A. First Formulation

One aspect of the invention provides an aqueous, ophthalmic solution, comprising:
a. from 4.7% (w/v) to 5.3% (w/v) of a compound of Formula I:

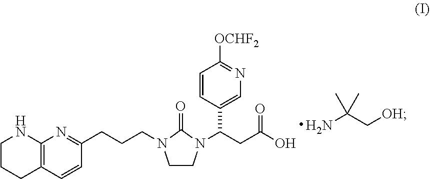

b. from 14% (w/v) to 18% (w/v) of a cyclodextrin;
c. from 0.1% (w/v) to 5% (w/v) of a buffer;
d. from 0.01% (w/v) to 1% (w/v) of a preservative; and
e. at least 75% (w/v) water;
wherein the solution has a pH in the range of 7.5 to 8.7.

The solution may be further characterized according to a variety of features, such as the identity of the cyclodextrin, the identity of the buffer, and the identity of the preservative. These and other features are described in more detail below.

### Identity & Amount of the Cyclodextrin

The solution may be further characterized according to the identity and/or amount of the cyclodextrin. For example, in certain embodiments, the cyclodextrin is 2-hydroxypropyl-β-cyclodextrin. In certain embodiments, the cyclodextrin is hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, or a mixture thereof. In certain embodiments, the cyclodextrin is β-cyclodextrin sulfobutyl ether, hydroxypropyl-β-cyclodextrin, sulphated β-cyclodextrin (S-β-CD), and maltosyl-β-cyclodextrin, or a mixture thereof. In certain embodiments, the cyclodextrin is β-cyclodextrin sulfobutyl ether or hydroxypropyl-β-cyclodextrin, or a mixture thereof.

In certain embodiments, the solution comprises from 15% (w/v) to 18% (w/v) of the cyclodextrin. In certain embodiments, the solution comprises from 15% (w/v) to 17% (w/v) of the cyclodextrin. In certain embodiments, the solution comprises from 15% (w/v) to 16% (w/v) of the cyclodextrin. In certain embodiments, the solution comprises from 15.2% (w/v) to 15.8% (w/v) of the cyclodextrin. In certain embodiments, the solution comprises from 15.3% (w/v) to 15.7% (w/v) of the cyclodextrin. In certain embodiments, the solution comprises from 15.4% (w/v) to 15.6% (w/v) of the cyclodextrin. In certain embodiments, the solution comprises 15.5% (w/v) of the cyclodextrin.

In certain embodiments, the cyclodextrin has a molecular weight in the range of from about 1200 g/mol to about 1600 g/mol. In certain embodiments, the cyclodextrin has a molecular weight in the range of from about 1300 g/mol to about 1500 g/mol. In certain embodiments, the cyclodextrin has a molecular weight in the range of from about 1350 g/mol to about 1450 g/mol. In certain embodiments, the cyclodextrin has a molecular weight of about 1400 g/mol.

In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.85. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.8. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.55 to about 0.77. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.59 to about 0.73.

### Buffer

The solution may be further characterized according to the identity and/or amount of the buffer. For example, in certain embodiments, the buffer comprises an organic acid. In certain embodiments, the buffer comprises boric acid. In certain embodiments, the buffer is a mixture of boric acid and an alkali metal borate. In certain embodiments, the buffer is a mixture of boric acid and sodium borate. In certain embodiments, the buffer comprises citric acid. In certain embodiments, the buffer is a mixture of citric acid and an alkali metal citrate. In certain embodiments, the buffer is a mixture of citric acid and sodium citrate.

In certain embodiments, the solution comprises from 0.1% (w/v) to 2.5% (w/v) of the buffer. In certain embodiments, the solution comprises from 0.25% (w/v) to 2.5% (w/v) of the buffer. In certain embodiments, the solution comprises from 0.5% (w/v) to 1.5% (w/v) of the buffer. In certain embodiments, the solution comprises from 0.75% (w/v) to 1.25% (w/v) of the buffer. In certain embodiments, the solution comprises from 0.8% (w/v) to 1.2% (w/v) of the buffer. In certain embodiments, the solution comprises from 0.9% (w/v) to 1.1% (w/v) of the buffer. In certain embodiments, the solution comprises 1% (w/v) of the buffer.

### Preservative

The solution may be further characterized according to the identity and/or amount of the preservative. For example, in certain embodiments, the preservative comprises a benzalkonium salt. In certain embodiments, the preservative comprises a benzalkonium halide. In certain embodiments, the preservative comprises benzalkonium chloride. In certain embodiments, the preservative is chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, sorbic acid, or a mixture thereof. In certain embodiments, the preservative is a benzalkonium halide (e.g., benzalkonium chloride), benzoxonium halide (e.g., benzoxonium chloride), chlorhexidine gluconate, benzethonium chloride, cetyl pyridinium chloride, potassium sorbate, sodium benzoate, ethyl p-hydroxybenzoate, butyl p-hydroxybenzoate, or propylaminopropyl biguanide, or a mixture thereof.

In certain embodiments, the solution comprises from 0.01% (w/v) to 0.1% (w/v) of the preservative. In certain embodiments, the solution comprises from 0.01% (w/v) to 0.05% (w/v) of the preservative. In certain embodiments, the solution comprises from 0.012% (w/v) to 0.05% (w/v) of the preservative. In certain embodiments, the solution comprises from 0.015% (w/v) to 0.05% (w/v) of the preservative. In certain embodiments, the solution comprises from 0.012% (w/v) to 0.03% (w/v) of the preservative. In certain embodiments, the solution comprises from 0.012% (w/v) to 0.025% (w/v) of the preservative. In certain embodiments, the solution comprises from 0.015% (w/v) to 0.025% (w/v) of the preservative. In certain embodiments, the solution comprises from 0.012% (w/v) to 0.02% (w/v) of the preservative. In certain embodiments, the solution comprises from 0.015% (w/v) to 0.02% (w/v) of the preservative. In certain embodiments, the solution comprises 0.02% (w/v) of the preservative.

### Chelating Agent

The solution may be further characterized according to the identity and/or amount of a chelating agent. For example, in certain embodiments, the solution further comprises a chelating agent.

In certain embodiments, the solution further comprises from 0.001% (w/v) to 2% (w/v) of a chelating agent. In certain embodiments, the solution further comprises from 0.01% (w/v) to 1% (w/v) of a chelating agent. In certain embodiments, the solution further comprises from 0.01% (w/v) to 0.5% (w/v) of a chelating agent. In certain embodiments, the solution further comprises from 0.05% (w/v) to 0.5% (w/v) of a chelating agent. In certain embodiments, the solution further comprises from 0.05% (w/v) to 0.1% (w/v) of a chelating agent. In certain embodiments, the solution further comprises from 0.01% (w/v) to 0.1% (w/v) of a chelating agent. In certain embodiments, the solution further comprises 0.1% (w/v) of a chelating agent.

In certain embodiments, the chelating agent comprises ethylenediaminetetraacetic acid or a salt thereof. In certain embodiments, the chelating agent is sodium ethylenediaminetetraacetate. In certain embodiments, the chelating agent is an alkali metal ethylenediaminetetraacetate.

### Water

The solution may be further characterized according to the amount of water in the solution. For example, in certain embodiments, the solution comprises at least 77% (w/v) water. In certain embodiments, the solution comprises at least 78% (w/v) water. In certain embodiments, the solution comprises from 70% (w/v) to 80% (w/v) water. In certain embodiments, the solution comprises from 75% (w/v) to 80% (w/v) water. In certain embodiments, the solution comprises from 75% (w/v) to 79% (w/v) water. In certain embodiments, the solution comprises from 75% (w/v) to 78.5% (w/v) water. In certain embodiments, the solution comprises from 75% (w/v) to 78.3% (w/v) water. In certain embodiments, the solution comprises from 77% (w/v) to 79% (w/v) water. In certain embodiments, the solution comprises from 77% (w/v) to 78% (w/v) water.

### pH of the Solution

The solution may be further characterized according to the pH of the solution. For example, in certain embodiments, the solution has a pH in the range of 7.5 to 8.5. In certain embodiments, the solution has a pH in the range of 7.8 to 8.5. In certain embodiments, the solution has a pH in the range of 7.8 to 8.2. In certain embodiments, the solution has a pH in the range of 7.9 to 8.1. In certain embodiments, the solution has a pH of 8.0. In certain embodiments, the solution has a pH of about 8.0. In certain embodiments, the solution has a pH in the range of about 7.5 to about 8.5.

### Tonicity Modifier

The solution may be further characterized according to the identity and/or amount of a tonicity modifier. For example, in certain embodiments, the solution further comprises a tonicity modifier. In certain embodiments, the solution further comprises about 0.01% (w/w) to about 5% (w/w) of a tonicity modifier. In certain embodiments, the solution further comprises about 0.1% (w/w) to about 2% (w/w) of a tonicity modifier.

### B. Second Formulation

Another aspect of the invention provides an aqueous, ophthalmic solution, comprising:
a. from 4.9% (w/v) to 5.1% (w/v) of a compound of Formula I:

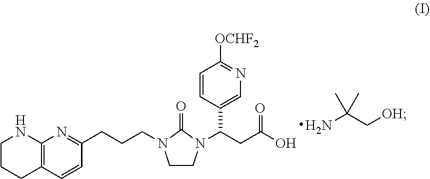

b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.2% (w/v) of a benzalkonium salt; and
e. at least 75% (w/v) water;
wherein the solution has a pH in the range of 7.8 to 8.5.

In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### C. Third Formulation

Another aspect of the invention provides an aqueous, ophthalmic solution, comprising:
a. 5.5% (w/v) of a compound of Formula I:

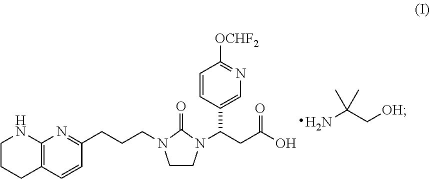

b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water;
wherein the solution has a pH in the range of 7.8 to 8.5.

In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### D. Fourth Formulation

Another aspect of the invention provides an aqueous, ophthalmic solution, comprising:
a. 5.5% (w/v) of a compound of Formula I:

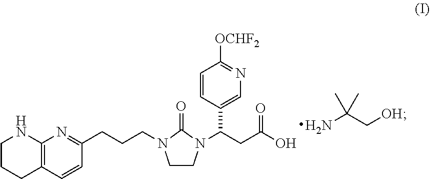

b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water;
wherein the solution has a pH in the range of 7.8 to 8.5.

In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### E. Fifth Formulation

Another aspect of the invention provides an aqueous, ophthalmic solution, consisting of:
a. from 4.9% (w/v) to 5.1% (w/v) of a compound of Formula I:

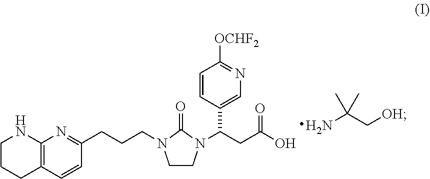

b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.5% (w/v) of a benzalkonium salt;
e. at least 75% (w/v) water; and
f. one or more excipients independently selected from the group consisting of a pH adjuster and a tonicity modifier;
wherein the solution has a pH in the range of 7.8 to 8.5.

In certain embodiments, the one or more excipients is a pH adjuster. In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### F. Sixth Formulation

Another aspect of the invention provides an aqueous, ophthalmic solution, consisting of:
a. 5.5% (w/v) of a compound of Formula I:

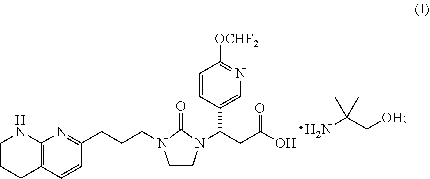

b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

In certain embodiments, the one or more excipients is a pH adjuster. In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### G. Seventh Formulation

Another aspect of the invention provides an aqueous, ophthalmic solution, consisting of:
a. 5.5% (w/v) of a compound of Formula I:

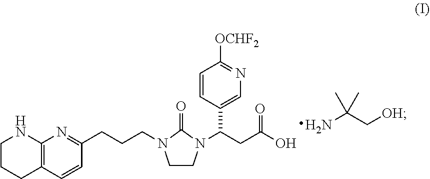

b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

In certain embodiments, the one or more excipients is a pH adjuster. In certain embodiments, benzalkonium salt is a benzalkonium halide. In certain embodiments, benzalkonium salt is benzalkonium chloride.

The solution may be further characterized according to the amount of 2-hydroxypropyl-β-cyclodextrin, amount of the buffer, amount of the benzalkonium salt, and other features as described in more detail below.

### H. Exemplary Further Embodiments for the Second Through Seventh Formulations

The Second through Seventh Formulations above may be further characterized according to features described herein below. For example, in certain embodiments, the 2-hydroxypropyl-β-cyclodextrin has a molecular weight in the range of from about 1300 g/mol to about 1500 g/mol. In certain embodiments, the 2-hydroxypropyl-β-cyclodextrin has a molecular weight in the range of from about 1350 g/mol to about 1450 g/mol. In certain embodiments, the 2-hydroxypropyl-β-cyclodextrin has a molecular weight of about 1400 g/mol.

In certain embodiments, the mole ratio of 2-hydroxylpropyl substituents per glucose moiety in the 2-hydroxypropyl-β-cyclodextrin is in the range of from about 0.5 to about 0.85. In certain embodiments, the mole ratio of 2-hydroxylpropyl substituents per glucose moiety in the 2-hydroxypropyl-β-cyclodextrin is in the range of from about 0.5 to about 0.8. In certain embodiments, the mole ratio of 2-hydroxylpropyl substituents per glucose moiety in the 2-hydroxypropyl-β-cyclodextrin is in the range of from about 0.55 to about 0.77. In certain embodiments, the mole ratio of 2-hydroxylpropyl substituents per glucose moiety in the 2-hydroxypropyl-β-cyclodextrin is in the range of from about 0.59 to about 0.73.

In certain embodiments, the solution has a pH in the range of 7.8 to 8.2. In certain embodiments, the solution has a pH of 8.0.

In certain embodiments, the solution has at least 77% (w/v) water. In certain embodiments, the solution has at least 78% (w/v) water.

### I. Exemplary Further Embodiments for the First Through Seventh Formulations

The First through Seventh Formulations above may be further characterized according to features described herein below. For example, in certain embodiments, less than 1% of the compound of Formula I degrades upon storage at 25° C. for 2 weeks. In certain embodiments, less than 0.5% of the compound of Formula I degrades upon storage at 25° C. for 2 weeks. In certain embodiments, less than 0.1% of the compound of Formula I degrades upon storage at 25° C. for 2 weeks. In certain embodiments, less than 1% of the compound of Formula I degrades upon storage at 25° C. for 24 weeks. In certain embodiments, less than 0.5% of the compound of Formula I degrades upon storage at 25° C. for 24 weeks. In certain embodiments, less than 1% of the compound of Formula I degrades upon storage at 40° C. for 2 weeks. In certain embodiments, less than 0.5% of the compound of Formula I degrades upon storage at 40° C. for 2 weeks. In certain embodiments, less than 0.1% of the compound of Formula I degrades upon storage at 40° C. for 2 weeks. In certain embodiments, less than 1% of the compound of Formula I degrades upon storage at 40° C. for 24 weeks. In certain embodiments, less than 0.5% of the compound of Formula I degrades upon storage at 40° C. for 24 weeks.

In certain embodiments, storage of the solution at 25° C. for 2 weeks results in a formulation containing less than 1% (w/w) of any solid precipitate that forms from the solution. In certain embodiments, storage of the solution at 25° C. for 24 weeks results in a formulation containing less than 1% (w/w) of any solid precipitate that forms from the solution. In certain embodiments, storage of the solution at 40° C. for 2 weeks results in a formulation containing less than 1% (w/w) of any solid precipitate that forms from the solution. In certain embodiments, storage of the solution at 40° C. for 24 weeks results in a formulation containing less than 1% (w/w) of any solid precipitate that forms from the solution. In certain embodiments, after storage of the solution at 25° C. for 2 weeks, there is no solid precipitate that forms from the solution. In certain embodiments, after storage of the solution at 25° C. for 24 weeks, there is no solid precipitate that forms from the solution. In certain embodiments, after storage of the solution at 40° C. for 2 weeks, there is no solid precipitate that forms from the solution. In certain embodiments, after storage of the solution at 40° C. for 24 weeks, there is no solid precipitate that forms from the solution. In certain embodiments, the solid precipitate comprises (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate. In certain embodiments, the solid precipitate is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

### J. Eighth Formulation

Another aspect of the invention provides an aqueous, ophthalmic solution, comprising:
a. from about 4% (w/v) to about 6% (w/v) of a compound of Formula II or a pharmaceutically acceptable salt thereof:

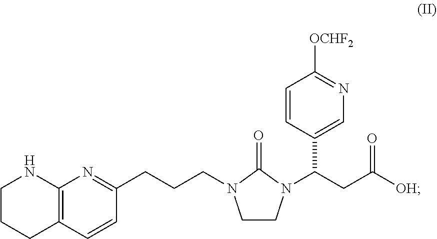 [SMILES: O=C(O)C[C@@H](c1ccc(OC(F)F)nc1)N1CCN(CCCc2ccc3c(n2)NCCC3)C1=O]

b. from 14% (w/v) to 18% (w/v) of a cyclodextrin;
c. a buffer;
d. a preservative; and
e. at least 75% (w/v) water;
wherein the solution has a pH in the range of 7.5 to 8.7.

The solution may be further characterized according to the identity and/or amount of cyclodextrin, buffer, preservative, and other features as described in more detail below.

For example, in certain embodiments, the identity and/or amount of cyclodextrin, buffer, and preservative are as set forth above for any one of the First through Seventh Formulations. In certain embodiments, the amount of water is as set forth above for any one of the First through Seventh Formulations. In certain embodiments, the pH of the solution is as set forth above for any one of the First through Seventh Formulations.

In certain embodiments, the cyclodextrin is 2-hydroxypropyl-β-cyclodextrin. In certain embodiments, the solution comprises from 15% (w/v) to 18% (w/v) of the cyclodextrin. In certain embodiments, the solution comprises from 15% (w/v) to 17% (w/v) of the cyclodextrin. In certain embodiments, the solution comprises from 15% (w/v) to 16% (w/v) of the cyclodextrin. In certain embodiments, the solution comprises from 15.2% (w/v) to 15.8% (w/v) of the cyclodextrin. In certain embodiments, the solution comprises 15.5% (w/v) of the cyclodextrin.

In certain embodiments, the buffer comprises boric acid.

In certain embodiments, the preservative comprises a benzalkonium salt.

### K. Nineth Formulation

Another aspect of the invention provides an aqueous solution, comprising:
a. a compound of Formula I:

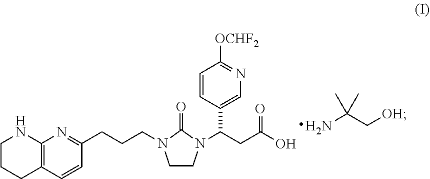

b. a cyclodextrin;
c. a buffer;
d. a preservative; and
e. at least 75% (w/v) water;
wherein the solution has a pH in the range of 7.5 to 8.7.

The solution may be further characterized according to the amount of compound of Formula I, identity and/or amount of cyclodextrin, buffer, benzalkonium salt, and other features as described in more detail below. In certain embodiments, the solution is an ophthalmic solution.

### Amount of Compound of Formula I

The solution may be further characterized according to amount of the compound of Formula I. For example, in certain embodiments, the solution comprises from about 0.5% (w/w) to about 7.5% (w/w) of a compound of Formula I. In certain embodiments, the solution comprises from about 0.5% (w/w) to about 1.0% (w/w) of a compound of Formula I. In certain embodiments, the solution comprises from about 1.0% (w/w) to about 1.25% (w/w) of a compound of Formula I. In certain embodiments, the solution comprises from about 1.25% (w/w) to about 2.5% (w/w) of a compound of Formula I. In certain embodiments, the solution comprises from about 2.5% (w/w) to about 5.0% (w/w) of a compound of Formula I. In certain embodiments, the solution comprises from about 5.0% (w/w) to about 7.5% (w/w) of a compound of Formula I. In certain embodiments, the solution comprises from about 0.3% (w/w) to about 0.7% (w/w) of a compound of Formula I. In certain embodiments, the solution comprises about 0.5% (w/w) of a compound of Formula I. In certain embodiments, the solution comprises from about 0.8% (w/w) to about 1.2% (w/w) of a compound of Formula I. In certain embodiments, the solution comprises about 1.0% (w/w) of a compound of Formula I. In certain embodiments, the solution comprises from about 1.05% (w/w) to about 1.45% (w/w) of a compound of Formula I. In certain embodiments, the solution comprises about 1.25% (w/w) of a compound of Formula I. In certain embodiments, the solution comprises from about 2.3% (w/w) to about 2.7% (w/w) of a compound of Formula I. In certain embodiments, the solution comprises about 2.5% (w/w) of a compound of Formula I. In certain embodiments, the solution comprises from about 4% (w/w) to about 6% (w/w) of a compound of Formula I. In certain embodiments, the solution comprises from about 4.5% (w/w) to about 5.5% (w/w) of a compound of Formula I. In certain embodiments, the solution comprises from about 4.8% (w/w) to about 5.2% (w/w) of a compound of Formula I. In certain embodiments, the solution comprises about 5.0% (w/w) of a compound of Formula I. In certain embodiments, the solution comprises from about 7% (w/w) to about 8% (w/w) of a compound of Formula I. In certain embodiments, the solution comprises from about 7.3% (w/w) to about 7.7% (w/w) of a compound of Formula I. In certain embodiments, the solution comprises about 7.5% (w/w) of a compound of Formula I.

In certain embodiments, the solution comprises from about 0.5% (w/v) to about 7.5% (w/v) of a compound of Formula I. In certain embodiments, the solution comprises from about 0.5% (w/v) to about 1.0% (w/v) of a compound of Formula I. In certain embodiments, the solution comprises from about 1.0% (w/v) to about 1.25% (w/v) of a compound of Formula I. In certain embodiments, the solution comprises from about 1.25% (w/v) to about 2.5% (w/v) of a compound of Formula I. In certain embodiments, the solution comprises from about 2.5% (w/v) to about 5.0% (w/v) of a compound of Formula I. In certain embodiments, the solution comprises from about 5.0% (w/v) to about 7.5% (w/v) of a compound of Formula I. In certain embodiments, the solution comprises from about 0.3% (w/v) to about 0.7% (w/v) of a compound of Formula I. In certain embodiments, the solution comprises about 0.5% (w/v) of a compound of Formula I. In certain embodiments, the solution comprises from about 0.8% (w/v) to about 1.2% (w/v) of a compound of Formula I. In certain embodiments, the solution comprises about 1.0% (w/v) of a compound of Formula I. In certain embodiments, the solution comprises from about 1.05% (w/v) to about 1.45% (w/v) of a compound of Formula I. In certain embodiments, the solution comprises about 1.25% (w/v) of a compound of Formula I. In certain embodiments, the solution comprises from about 2.3% (w/v) to about 2.7% (w/v) of a compound of Formula I. In certain embodiments, the solution comprises about 2.5% (w/v) of a compound of Formula I. In certain embodiments, the solution comprises from about 4% (w/v) to about 6% (w/v) of a compound of Formula I. In certain embodiments, the solution comprises from about 4.5% (w/v) to about 5.5% (w/v) of a compound of Formula I. In certain embodiments, the solution comprises from about 4.8% (w/v) to about 5.2% (w/v) of a compound of Formula I. In certain embodiments, the solution comprises about 5.0% (w/v) of a compound of Formula I. In certain embodiments, the solution comprises from about 7% (w/v) to about 8% (w/v) of a compound of Formula I. In certain embodiments, the solution comprises from about 7.3% (w/v) to about 7.7% (w/v) of a compound of Formula I. In certain embodiments, the solution comprises about 7.5% (w/v) of a compound of Formula I.

### Identity & Amount of the Cyclodextrin

The solution may be further characterized according to the identity and/or amount of the cyclodextrin. For example, in certain embodiments, the cyclodextrin is 2-hydroxypropyl-β-cyclodextrin. In certain embodiments, the cyclodextrin is hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, or a mixture thereof. In certain embodiments, the cyclodextrin is β-cyclodextrin sulfobutyl ether, hydroxypropyl-β-cyclodextrin, sulphated β-cyclodextrin (S-β-CD), and maltosyl-β-cyclodextrin, or a mixture thereof. In certain embodiments, the cyclodextrin is β-cyclodextrin sulfobutyl ether or hydroxypropyl-β-cyclodextrin, or a mixture thereof.

In certain embodiments, the mole ratio of cyclodextrin to compound of Formula I is at least 1.25:1. In certain embodiments, the mole ratio of cyclodextrin to compound of Formula I is from 1.25:1 to 2:1. In certain embodiments, the mole ratio of cyclodextrin to compound of Formula I is from 1.25:1 to 1.75:1. In certain embodiments, the mole ratio of cyclodextrin to compound of Formula I is from 1.25:1 to 1.5:1. In certain embodiments, the mole ratio of cyclodextrin to compound of Formula I is from 1.25:1 to 1.45:1. In certain embodiments, the mole ratio of cyclodextrin to compound of Formula I is from 1.25:1 to 1.4:1. In certain embodiments, the mole ratio of cyclodextrin to compound of Formula I is from 1.25:1 to 1.35:1. In certain embodiments, the mole ratio of cyclodextrin to compound of Formula I is from 1.25:1 to 1.3:1.

In certain embodiments, the solution comprises from 15% (w/v) to 18% (w/v) of the cyclodextrin. In certain embodiments, the solution comprises from 15% (w/v) to 17% (w/v) of the cyclodextrin. In certain embodiments, the solution comprises from 15% (w/v) to 16% (w/v) of the cyclodextrin. In certain embodiments, the solution comprises from 15.2% (w/v) to 15.8% (w/v) of the cyclodextrin. In certain embodiments, the solution comprises from 15.3% (w/v) to 15.7% (w/v) of the cyclodextrin. In certain embodiments, the solution comprises from 15.4% (w/v) to 15.6% (w/v) of the cyclodextrin. In certain embodiments, the solution comprises 15.5% (w/v) of the cyclodextrin.

In certain embodiments, the cyclodextrin has a molecular weight in the range of from about 1200 g/mol to about 1600 g/mol. In certain embodiments, the cyclodextrin has a molecular weight in the range of from about 1300 g/mol to about 1500 g/mol. In certain embodiments, the cyclodextrin has a molecular weight in the range of from about 1350 g/mol to about 1450 g/mol. In certain embodiments, the cyclodextrin has a molecular weight of about 1400 g/mol. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.85. In certain embodiments, the cyclodextrin is a 2-hydroxypropyl-β-cyclodextrin in which the of 2-hydroxylpropyl substituents per glucose moiety in the β-cyclodextrin is in the range of from about 0.5 to about 0.8. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.55 to about 0.77. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.59 to about 0.73.

### Buffer

The solution may be further characterized according to the identity and/or amount of the buffer. For example, in certain embodiments, the buffer comprises an organic acid. In certain embodiments, the buffer comprises boric acid. In certain embodiments, the buffer is a mixture of boric acid and an alkali metal borate. In certain embodiments, the buffer is a mixture of boric acid and sodium borate. In certain embodiments, the buffer comprises citric acid. In certain embodiments, the buffer is a mixture of citric acid and an alkali metal citrate. In certain embodiments, the buffer is a mixture of citric acid and sodium citrate.

In certain embodiments, the solution comprises from 0.1% (w/v) to 2.5% (w/v) of the buffer. In certain embodiments, the solution comprises from 0.25% (w/v) to 2.5% (w/v) of the buffer. In certain embodiments, the solution comprises from 0.5% (w/v) to 1.5% (w/v) of the buffer. In certain embodiments, the solution comprises from 0.75% (w/v) to 1.25% (w/v) of the buffer. In certain embodiments, the solution comprises from 0.8% (w/v) to 1.2% (w/v) of the buffer. In certain embodiments, the solution comprises from 0.9% (w/v) to 1.1% (w/v) of the buffer. In certain embodiments, the solution comprises 1% (w/v) of the buffer.

### Preservative

The solution may be further characterized according to the identity and/or amount of the preservative. For example, in certain embodiments, the preservative comprises a benzalkonium salt. In certain embodiments, the preservative comprises a benzalkonium halide. In certain embodiments, the preservative comprises benzalkonium chloride. In certain embodiments, the preservative is chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, sorbic acid, or a mixture thereof. In certain embodiments, the preservative is a benzalkonium halide (e.g., benzalkonium chloride), benzoxonium halide (e.g., benzoxonium chloride), chlorhexidine gluconate, benzethonium chloride, cetyl pyridinium chloride, potassium sorbate, sodium benzoate, ethyl p-hydroxybenzoate, butyl p-hydroxybenzoate, or propylaminopropyl biguanide, or a mixture thereof.

In certain embodiments, the solution comprises from 0.01% (w/v) to 0.1% (w/v) of the preservative. In certain embodiments, the solution comprises from 0.01% (w/v) to 0.05% (w/v) of the preservative. In certain embodiments, the solution comprises from 0.012% (w/v) to 0.05% (w/v) of the preservative. In certain embodiments, the solution comprises from 0.015% (w/v) to 0.05% (w/v) of the preservative. In certain embodiments, the solution comprises from 0.012% (w/v) to 0.03% (w/v) of the preservative. In certain embodiments, the solution comprises from 0.012% (w/v) to 0.025% (w/v) of the preservative. In certain embodiments, the solution comprises from 0.015% (w/v) to 0.025% (w/v) of the preservative. In certain embodiments, the solution comprises from 0.012% (w/v) to 0.02% (w/v) of the preservative. In certain embodiments, the solution comprises from 0.015% (w/v) to 0.02% (w/v) of the preservative. In certain embodiments, the solution comprises 0.02% (w/v) of the preservative.

### Chelating Agent

The solution may be further characterized according to the identity and/or amount of a chelating agent. For example, in certain embodiments, the solution further comprises a chelating agent.

In certain embodiments, the solution further comprises from 0.001% (w/v) to 2% (w/v) of a chelating agent. In certain embodiments, the solution further comprises from 0.01% (w/v) to 1% (w/v) of a chelating agent. In certain embodiments, the solution further comprises from 0.01% (w/v) to 0.5% (w/v) of a chelating agent. In certain embodiments, the solution further comprises from 0.05% (w/v) to 0.5% (w/v) of a chelating agent. In certain embodiments, the solution further comprises from 0.05% (w/v) to 0.1% (w/v) of a chelating agent. In certain embodiments, the solution further comprises from 0.01% (w/v) to 0.1% (w/v) of a chelating agent. In certain embodiments, the solution further comprises 0.1% (w/v) of a chelating agent.

In certain embodiments, the chelating agent comprises ethylenediaminetetraacetic acid or a salt thereof. In certain embodiments, the chelating agent is sodium ethylenediaminetetraacetate.

### Water

The solution may be further characterized according to the amount of water in the solution. For example, in certain embodiments, the solution comprises at least 77% (w/v) water. In certain embodiments, the solution comprises at least 78% (w/v) water. In certain embodiments, the solution comprises from 70% (w/v) to 80% (w/v) water. In certain embodiments, the solution comprises from 75% (w/v) to 80% (w/v) water. In certain embodiments, the solution comprises from 75% (w/v) to 79% (w/v) water. In certain embodiments, the solution comprises from 75% (w/v) to 78.5% (w/v) water. In certain embodiments, the solution comprises from 75% (w/v) to 78.3% (w/v) water. In certain embodiments, the solution comprises from 77% (w/v) to 79% (w/v) water. In certain embodiments, the solution comprises from 77% (w/v) to 78% (w/v) water.

### pH of the Solution

The solution may be further characterized according to the pH of the solution. For example, in certain embodiments, the solution has a pH in the range of 7.5 to 8.5. In certain embodiments, the solution has a pH in the range of 7.8 to 8.5. In certain embodiments, the solution has a pH in the range of 7.8 to 8.2. In certain embodiments, the solution has a pH in the range of 7.9 to 8.1. In certain embodiments, the solution has a pH of 8.0.

### Additional Characterization

In certain embodiments, the amount of compound of Formula I and/or the identity and/or amount of cyclodextrin, buffer, and preservative are as set forth above for any one of the First through Seventh Formulations. In certain embodiments, the amount of water is as set forth above for any one of the First through Seventh Formulations. In certain embodiments, the pH of the solution is as set forth above for any one of the First through Seventh Formulations. In certain embodiments, the solution is further characterized according to one or more features set forth above for any one of the First through Seventh Formulations.

### L. Tenth Formulation

Another aspect of the invention provides an aqueous solution, comprising:
a. a compound of Formula II or a pharmaceutically acceptable salt thereof:

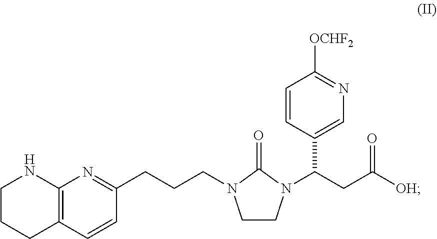 [SMILES: O=C(O)C[C@@H](c1ccc(OC(F)F)nc1)N1CCN(CCCc2ccc3c(n2)NCCC3)C1=O]

b. a cyclodextrin;
c. a buffer;
d. a preservative; and
e. water.

The solution may be further characterized according to the identity and/or amount of cyclodextrin, buffer, benzalkonium salt, and other features as described in more detail below. In certain embodiments, the solution is an ophthalmic solution.

### Amount of Compound of Formula II

The solution may be further characterized according to amount of the compound of Formula II. For example, in certain embodiments, the solution comprises from about 0.5% (w/w) to about 7.5% (w/w) of a compound of Formula II. In certain embodiments, the solution comprises from about 0.5% (w/w) to about 1.0% (w/w) of a compound of Formula II. In certain embodiments, the solution comprises from about 1.0% (w/w) to about 1.25% (w/w) of a compound of Formula II. In certain embodiments, the solution comprises from about 1.25% (w/w) to about 2.5% (w/w) of a compound of Formula II. In certain embodiments, the solution comprises from about 2.5% (w/w) to about 5.0% (w/w) of a compound of Formula II. In certain embodiments, the solution comprises from about 5.0% (w/w) to about 7.5% (w/w) of a compound of Formula II. In certain embodiments, the solution comprises from about 0.3% (w/w) to about 0.7% (w/w) of a compound of Formula II. In certain embodiments, the solution comprises about 0.5% (w/w) of a compound of Formula II. In certain embodiments, the solution comprises from about 0.8% (w/w) to about 1.2% (w/w) of a compound of Formula II. In certain embodiments, the solution comprises about 1.0% (w/w) of a compound of Formula II. In certain embodiments, the solution comprises from about 1.05% (w/w) to about 1.45% (w/w) of a compound of Formula II. In certain embodiments, the solution comprises about 1.25% (w/w) of a compound of Formula II. In certain embodiments, the solution comprises from about 2.3% (w/w) to about 2.7% (w/w) of a compound of Formula II. In certain embodiments, the solution comprises about 2.5% (w/w) of a compound of Formula II. In certain embodiments, the solution comprises from about 4% (w/w) to about 6% (w/w) of a compound of Formula II. In certain embodiments, the solution comprises from about 4.5% (w/w) to about 5.5% (w/w) of a compound of Formula II. In certain embodiments, the solution comprises from about 4.8% (w/w) to about 5.2% (w/w) of a compound of Formula II. In certain embodiments, the solution comprises about 5.0% (w/w) of a compound of Formula II. In certain embodiments, the solution comprises from about 7% (w/w) to about 8% (w/w) of a compound of Formula II. In certain embodiments, the solution comprises from about 7.3% (w/w) to about 7.7% (w/w) of a compound of Formula II. In certain embodiments, the solution comprises about 7.5% (w/w) of a compound of Formula II.

In certain embodiments, the solution comprises from about 0.5% (w/v) to about 7.5% (w/v) of a compound of Formula II. In certain embodiments, the solution comprises from about 0.5% (w/v) to about 1.0% (w/v) of a compound of Formula II. In certain embodiments, the solution comprises from about 1.0% (w/v) to about 1.25% (w/v) of a compound of Formula II. In certain embodiments, the solution comprises from about 1.25% (w/v) to about 2.5% (w/v) of a compound of Formula II. In certain embodiments, the solution comprises from about 2.5% (w/v) to about 5.0% (w/v) of a compound of Formula II. In certain embodiments, the solution comprises from about 5.0% (w/v) to about 7.5% (w/v) of a compound of Formula II. In certain embodiments, the solution comprises from about 0.3% (w/v) to about 0.7% (w/v) of a compound of Formula II. In certain embodiments, the solution comprises about 0.5% (w/v) of a compound of Formula II. In certain embodiments, the solution comprises from about 0.8% (w/v) to about 1.2% (w/v) of a compound of Formula II. In certain embodiments, the solution comprises about 1.0% (w/v) of a compound of Formula II. In certain embodiments, the solution comprises from about 1.05% (w/v) to about 1.45% (w/v) of a compound of Formula II. In certain embodiments, the solution comprises about 1.25% (w/v) of a compound of Formula II. In certain embodiments, the solution comprises from about 2.3% (w/v) to about 2.7% (w/v) of a compound of Formula II. In certain embodiments, the solution comprises about 2.5% (w/v) of a compound of Formula II. In certain embodiments, the solution comprises from about 4% (w/v) to about 6% (w/v) of a compound of Formula II. In certain embodiments, the solution comprises from about 4.5% (w/v) to about 5.5% (w/v) of a compound of Formula II. In certain embodiments, the solution comprises from about 4.8% (w/v) to about 5.2% (w/v) of a compound of Formula II. In certain embodiments, the solution comprises about 5.0% (w/v) of a compound of Formula II. In certain embodiments, the solution comprises from about 7% (w/v) to about 8% (w/v) of a compound of Formula II. In certain embodiments, the solution comprises from about 7.3% (w/v) to about 7.7% (w/v) of a compound of Formula II. In certain embodiments, the solution comprises about 7.5% (w/v) of a compound of Formula II.

In certain embodiments the compound of Formula II is a 2-amino-2-methylpropan-1-ol salt of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid.

### Identity & Amount of the Cyclodextrin

The solution may be further characterized according to the identity and/or amount of the cyclodextrin. For example, in certain embodiments, the cyclodextrin is 2-hydroxypropyl-β-cyclodextrin. In certain embodiments, the cyclodextrin is hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, or a mixture thereof. In certain embodiments, the cyclodextrin is β-cyclodextrin sulfobutyl ether, hydroxypropyl-β-cyclodextrin, sulphated β-cyclodextrin (S-β-CD), and maltosyl-β-cyclodextrin, or a mixture thereof. In certain embodiments, the cyclodextrin is β-cyclodextrin sulfobutyl ether or hydroxypropyl-β-cyclodextrin, or a mixture thereof.

In certain embodiments, the mole ratio of cyclodextrin to compound of Formula II is at least 1.25:1. In certain embodiments, the mole ratio of cyclodextrin to compound of Formula II is from 1.25:1 to 2:1. In certain embodiments, the mole ratio of cyclodextrin to compound of Formula II is from 1.25:1 to 1.75:1. In certain embodiments, the mole ratio of cyclodextrin to compound of Formula II is from 1.25:1 to 1.5:1. In certain embodiments, the mole ratio of cyclodextrin to compound of Formula II is from 1.25:1 to 1.45:1. In certain embodiments, the mole ratio of cyclodextrin to compound of Formula II is from 1.25:1 to 1.4:1. In certain embodiments, the mole ratio of cyclodextrin to compound of Formula II is from 1.25:1 to 1.35:1. In certain embodiments, the mole ratio of cyclodextrin to compound of Formula II is from 1.25:1 to 1.3:1.

In certain embodiments, the solution comprises from 15% (w/v) to 18% (w/v) of the cyclodextrin. In certain embodiments, the solution comprises from 15% (w/v) to 17% (w/v) of the cyclodextrin. In certain embodiments, the solution comprises from 15% (w/v) to 16% (w/v) of the cyclodextrin. In certain embodiments, the solution comprises from 15.2% (w/v) to 15.8% (w/v) of the cyclodextrin. In certain embodiments, the solution comprises from 15.3% (w/v) to 15.7% (w/v) of the cyclodextrin. In certain embodiments, the solution comprises from 15.4% (w/v) to 15.6% (w/v) of the cyclodextrin. In certain embodiments, the solution comprises 15.5% (w/v) of the cyclodextrin.

In certain embodiments, the cyclodextrin has a molecular weight in the range of from about 1200 g/mol to about 1600 g/mol. In certain embodiments, the cyclodextrin has a molecular weight in the range of from about 1300 g/mol to about 1500 g/mol. In certain embodiments, the cyclodextrin has a molecular weight in the range of from about 1350 g/mol to about 1450 g/mol. In certain embodiments, the cyclodextrin has a molecular weight of about 1400 g/mol. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.85. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.8. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.55 to about 0.77. In certain embodiments, the cyclodextrin is a 2-hydroxypropyl-β-cyclodextrin in which the mole ratio of 2-hydroxylpropyl substituents per glucose moiety in the β-cyclodextrin is in the range of from about 0.59 to about 0.73.

### Buffer

The solution may be further characterized according to the identity and/or amount of the buffer. For example, in certain embodiments, the buffer comprises an organic acid. In certain embodiments, the buffer comprises boric acid. In certain embodiments, the buffer is a mixture of boric acid and an alkali metal borate. In certain embodiments, the buffer is a mixture of boric acid and sodium borate. In certain embodiments, the buffer comprises citric acid. In certain embodiments, the buffer is a mixture of citric acid and an alkali metal citrate. In certain embodiments, the buffer is a mixture of citric acid and sodium citrate.

In certain embodiments, the solution comprises from 0.1% (w/v) to 2.5% (w/v) of the buffer. In certain embodiments, the solution comprises from 0.25% (w/v) to 2.5% (w/v) of the buffer. In certain embodiments, the solution comprises from 0.5% (w/v) to 1.5% (w/v) of the buffer. In certain embodiments, the solution comprises from 0.75% (w/v) to 1.25% (w/v) of the buffer. In certain embodiments, the solution comprises from 0.8% (w/v) to 1.2% (w/v) of the buffer. In certain embodiments, the solution comprises from 0.9% (w/v) to 1.1% (w/v) of the buffer. In certain embodiments, the solution comprises 1% (w/v) of the buffer.

### Preservative

The solution may be further characterized according to the identity and/or amount of the preservative. For example, in certain embodiments, the preservative comprises a benzalkonium salt. In certain embodiments, the preservative comprises a benzalkonium halide. In certain embodiments, the preservative comprises benzalkonium chloride. In certain embodiments, the preservative is chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, sorbic acid, or a mixture thereof. In certain embodiments, the preservative is a benzalkonium halide (e.g., benzalkonium chloride), benzoxonium halide (e.g., benzoxonium chloride), chlorhexidine gluconate, benzethonium chloride, cetyl pyridinium chloride, potassium sorbate, sodium benzoate, ethyl p-hydroxybenzoate, butyl p-hydroxybenzoate, or propylaminopropyl biguanide, or a mixture thereof.

In certain embodiments, the solution comprises from 0.01% (w/v) to 0.1% (w/v) of the preservative. In certain embodiments, the solution comprises from 0.01% (w/v) to 0.05% (w/v) of the preservative. In certain embodiments, the solution comprises from 0.012% (w/v) to 0.05% (w/v) of the preservative. In certain embodiments, the solution comprises from 0.015% (w/v) to 0.05% (w/v) of the preservative. In certain embodiments, the solution comprises from 0.012% (w/v) to 0.03% (w/v) of the preservative. In certain embodiments, the solution comprises from 0.012% (w/v) to 0.025% (w/v) of the preservative. In certain embodiments, the solution comprises from 0.015% (w/v) to 0.025% (w/v) of the preservative. In certain embodiments, the solution comprises from 0.012% (w/v) to 0.02% (w/v) of the preservative. In certain embodiments, the solution comprises from 0.015% (w/v) to 0.02% (w/v) of the preservative. In certain embodiments, the solution comprises 0.02% (w/v) of the preservative.

### Chelating Agent

The solution may be further characterized according to the identity and/or amount of a chelating agent. For example, in certain embodiments, the solution further comprises a chelating agent.

In certain embodiments, the solution further comprises from 0.001% (w/v) to 2% (w/v) of a chelating agent. In certain embodiments, the solution further comprises from 0.01% (w/v) to 1% (w/v) of a chelating agent. In certain embodiments, the solution further comprises from 0.01% (w/v) to 0.5% (w/v) of a chelating agent. In certain embodiments, the solution further comprises from 0.05% (w/v) to 0.5% (w/v) of a chelating agent. In certain embodiments, the solution further comprises from 0.05% (w/v) to 0.1% (w/v) of a chelating agent. In certain embodiments, the solution further comprises from 0.01% (w/v) to 0.1% (w/v) of a chelating agent. In certain embodiments, the solution further comprises 0.1% (w/v) of a chelating agent.

In certain embodiments, the chelating agent comprises ethylenediaminetetraacetic acid or a salt thereof. In certain embodiments, the chelating agent is sodium ethylenediaminetetraacetate.

### Water

The solution may be further characterized according to the amount of water in the solution. For example, in certain embodiments, the solution comprises at least 77% (w/v) water. In certain embodiments, the solution comprises at least 78% (w/v) water.

### pH of the Solution

The solution may be further characterized according to the pH of the solution. For example, in certain embodiments, the solution has a pH in the range of 7.5 to 8.5. In certain embodiments, the solution has a pH in the range of 7.8 to 8.5. In certain embodiments, the solution has a pH in the range of 7.8 to 8.2. In certain embodiments, the solution has a pH in the range of 7.9 to 8.1. In certain embodiments, the solution has a pH of 8.0.

### Additional Characterization

In certain embodiments, the identity and/or amount of cyclodextrin, buffer, and preservative are as set forth above for any one of the First through Seventh Formulations. In certain embodiments, the amount of water is as set forth above for any one of the First through Seventh Formulations. In certain embodiments, the pH of the solution is as set forth above for any one of the First through Seventh Formulations. In certain embodiments, the solution is further characterized according to one or more features set forth above for any one of the First through Seventh Formulations.

### N. Eleventh Formulation

Another aspect of the invention provides an aqueous solution, comprising:
a. a compound of Formula II or a pharmaceutically acceptable salt thereof:

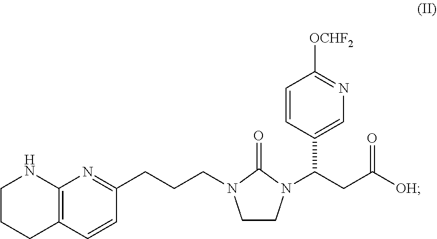 [SMILES: O=C(O)C[C@@H](c1ccc(OC(F)F)nc1)N1CCN(CCCc2ccc3c(n2)NCCC3)C1=O]

b. a cyclodextrin; and
c. water.

The solution may be further characterized according to the identity and/or amount of the compound of Formula II, cyclodextrin and any buffer and/or benzalkonium salt, and other features. In certain embodiments, the identity and/or amount of cyclodextrin, buffer, and preservative are as set forth above for any one of the First through Seventh Formulations. In certain embodiments, the amount of water is as set forth above for any one of the First through Seventh Formulations. In certain embodiments, the pH of the solution is as set forth above for any one of the First through Seventh Formulations. In certain embodiments, the solution is further characterized according to one or more features set forth above for any one of the First through Tenth Formulations. In certain embodiments, the solution is further characterized according to one or more features set forth above for the Twelfth Formulation.

In certain embodiments, the solution is an ophthalmic solution.

### M. Twelfth Formulation

Another aspect of the invention relates to a formulation of an αv integrin antagonist having the structure:

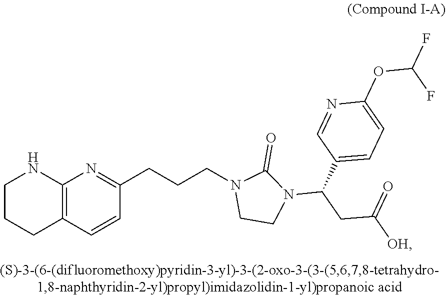 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof. The formulation may further comprise a pharmaceutically acceptable carrier or excipient. In one embodiment, the formulation of Compound I-A is for topical administration to the eye.

In an embodiment, the pharmaceutically acceptable salt of Compound I-A is the 1-hydroxy-2-methylpropan-2-aminium salt having the structure:

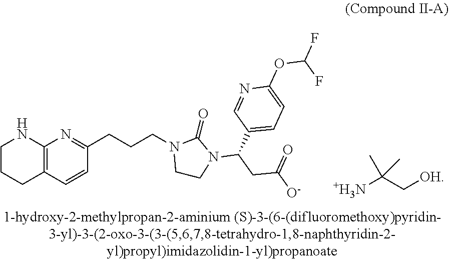

In one embodiment, the formulation of the present application comprises Compound I-A, or a pharmaceutically acceptable salt or solvate thereof (e.g., Compound II-A), and a solubility enhancing agent, and optionally one or more additional pharmaceutically acceptable carriers or excipients. In a further embodiment, the solubility enhancing agent is a cyclodextrin. In a further embodiment, the cyclodextrin is hydroxypropyl-β-cyclodextrin (HPβCD) or sulfobutyl ether β-cyclodextrin. In a further embodiment, the cyclodextrin is HPβCD.

In one embodiment, the formulation of the present application comprises Compound II-A, and a solubility enhancing agent, and optionally one or more additional pharmaceutically acceptable carriers or excipients. In a further embodiment, the solubility enhancing agent is a cyclodextrin. In a further embodiment, the cyclodextrin is HPβCD or sulfobutyl ether β-cyclodextrin. In a further embodiment, the cyclodextrin is HPβCD.

For topical ocular administration, the formulations are provided as an ophthalmic formulation comprising Compound I-A, or a pharmaceutically acceptable salt thereof (e.g., Compound II-A).

Additional exemplary embodiments are provided herein below:

(A1) In one embodiment, the formulations provided herein comprise Compound I-A, or a pharmaceutically acceptable salt thereof (e.g., Compound II-A), in an amount between about 0.05% w/w and about 30% w/w, between about 0.1% w/w and about 15.0% w/w, between about 0.2% w/w and about 10.0% w/w, between about 0.3% w/w and about 8.0% w/w, between about 0.4% w/w and about 6.0% w/w, between about 0.5% w/w and about 5.5% w/w, between about 1% w/w and about 5.0% w/w, between about 0.5% w/w and about 1.5% w/w, between about 2.0% w/w and about 3.0% w/w, or between about 4.5% w/w and about 5.5% w/w.

(A2) In one embodiment, the formulations provided herein comprise Compound I-A, or a pharmaceutically acceptable salt thereof (e.g., Compound II-A), in an amount between about 0.05% w/w and about 0.5% w/w, between about 0.5% w/w and about 1% w/w, between about 1% w/w and about 1.25% w/w, between about 1.25% w/w and about 1.5% w/w, between about 1.5% w/w and about 1.75% w/w, between about 1.75% w/w and about 2.0% w/w, between about 2.0% w/w and about 2.5% w/w, between about 2.5% w/w and about 3.0% w/w, between about 3.0% w/w and about 3.5% w/w, between about 3.5% w/w and about 4.0% w/w, between about 4.5% w/w and about 5.0% w/w, between about 5.0% w/w and about 5.5% w/w, between about 5.5% w/w and about 6.0% w/w, between about 6.0% w/w and about 6.5% w/w, between about 6.5% w/w and about 7.0% w/w, between about 7.0% w/w and about 8.0% w/w, between about 8.0% w/w and about 9.0% w/w, between about 9.0% w/w and about 10.0% w/w, between about 10.0% w/w and about 11.0% w/w, between about 11.0% w/w and about 12.0% w/w, between about 12.0% w/w and about 13.0% w/w, between about 13.0% w/w and about 14.0% w/w, between about 14.0% w/w and about 15.0% w/w, between about 15.0% w/w and about 16.0% w/w, between about 16.0% w/w and about 18.0% w/w, between about 18.0% w/w and about 20.0% w/w, between about 20.0% w/w and about 22.0% w/w, between about 22.0% w/w and about 24.0% w/w, between about 24.0% w/w and about 26.0% w/w, between about 26.0% w/w and about 28.0% w/w, or between about 28.0% w/w and about 30.0% w/w, or any combination thereof (e.g., between about 1.5% w/w and about 6.0% w/w, between about 1.5% w/w and about 10.0% w/w, or between about 1.5% w/w and about 20.0% w/w, etc.).

(A3) In one embodiment, the formulations provided herein comprise Compound I-A, or a pharmaceutically acceptable salt thereof (e.g., Compound II-A), in an amount of about 0.5% w/w, about 1.0% w/w, about 1.1% w/w, about 1.2% w/w, about 1.25% w/w, about 1.3% w/w, about 1.4% w/w, about 1.5% w/w, about 1.6% w/w, about 1.7% w/w, about 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5.0% w/w, about 5.1% w/w, about 5.2% w/w, about 5.3% w/w, about 5.4% w/w, about 5.5% w/w, about 6.0% w/w, about 6.5% w/w, about 7.0% w/w, about 7.5% w/w, about 8.0% w/w, about 8.5% w/w, about 9.0% w/w, about 9.5% w/w, about 10% w/w, about 11% w/w, about 12% w/w, about 12.5% w/w, about 13% w/w, about 14% w/w, about 15% w/w, about 16% w/w, about 17% w/w, about 17.5% w/w, about 18% w/w, about 19% w/w, or about 20% w/w.

(A4) In one embodiment, the formulations provided herein comprise Compound I-A, or a pharmaceutically acceptable salt thereof (e.g., Compound II-A), in an amount of about 0.5% w/w, about 1.25% w/w, about 2.5% w/w, about 5.0% w/w, or about 7.5% w/w.

(A5) In one embodiment, the formulations provided herein comprise Compound I-A, or a pharmaceutically acceptable salt thereof (e.g., Compound II-A), in an amount between about 7.0% w/w and about 8.0% w/w, between about 7.0% w/w and about 7.9% w/w, between about 7.0% w/w and about 7.8% w/w, between about 7.0% w/w and about 7.7% w/w, between about 7.0% w/w and about 7.6% w/w, between about 7.1% w/w and about 8.0% w/w, between about 7.1% w/w and about 7.9% w/w, between about 7.1% w/w and about 7.8% w/w, between about 7.1% w/w and about 7.7% w/w, between about 7.1% w/w and about 7.6% w/w, between about 7.2% w/w and about 8.0% w/w, between about 7.2% w/w and about 7.9% w/w, between about 7.2% w/w and about 7.8% w/w, between about 7.2% w/w and about 7.7% w/w, between about 7.2% w/w and about 7.6% w/w, between about 7.3% w/w and about 8.0% w/w, between about 7.3% w/w and about 7.9% w/w, between about 7.3% w/w and about 7.8% w/w, between about 7.3% w/w and about 7.7% w/w, between about 7.3% w/w and about 7.6% w/w, between about 7.4% w/w and about 8.0% w/w, between about 7.4% w/w and about 7.9% w/w, between about 7.4% w/w and about 7.8% w/w, between about 7.4% w/w and about 7.7% w/w, or between about 7.4% w/w and about 7.6% w/w (e.g., about 7.5% w/w).

(A6) In one embodiment, the formulations provided herein comprise Compound I-A, or a pharmaceutically acceptable salt thereof (e.g., Compound II-A), in an amount between about 4.2% w/w and about 5.2% w/w, between about 4.2% w/w and about 5.1% w/w, between about 4.2% w/w and about 5.0% w/w, between about 4.2% w/w and about 4.9% w/w, between about 4.2% w/w and about 4.8% w/w, between about 4.3% w/w and about 5.2% w/w, between about 4.3% w/w and about 5.1% w/w, between about 4.3% w/w and about 5.0% w/w, between about 4.3% w/w and about 4.9% w/w, between about 4.3% w/w and about 4.8% w/w, between about 4.4% w/w and about 5.2% w/w, between about 4.4% w/w and about 5.1% w/w, between about 4.4% w/w and about 5.0% w/w, between about 4.4% w/w and about 4.9% w/w, between about 4.4% w/w and about 4.8% w/w, between about 4.5% w/w and about 5.2% w/w, 4.5% w/w and about 5.1% w/w, 4.5% w/w and about 5.0% w/w, between about 4.5% w/w and about 4.9% w/w, between about 4.5% w/w and about 4.8% w/w, between about 4.6% w/w and about 5.2% w/w, 4.6% w/w and about 5.1% w/w, 4.6% w/w and about 5.0% w/w, between about 4.6% w/w and about 4.9% w/w, or between about 4.6% w/w and about 4.8% w/w (e.g., about 4.7% w/w).

(A7) In one embodiment, the formulations provided herein comprise Compound I-A, or a pharmaceutically acceptable salt thereof (e.g., Compound II-A), in an amount between about 1.9% w/w and about 2.9% w/w, between about 1.9% w/w and about 2.8% w/w, between about 1.9% w/w and about 2.7% w/w, between about 1.9% w/w and about 2.6% w/w, between about 1.9% w/w and about 2.5% w/w, between about 2.0% w/w and about 2.9% w/w, between about 2.0% w/w and about 2.8% w/w, between about 2.0% w/w and about 2.7% w/w, between about 2.0% w/w and about 2.6% w/w, between about 2.0% w/w and about 2.5% w/w, between about 2.1% w/w and about 2.9% w/w, between about 2.1% w/w and about 2.8% w/w, between about 2.1% w/w and about 2.7% w/w, between about 2.1% w/w and about 2.6% w/w, between about 2.1% w/w and about 2.5% w/w, between about 2.2% w/w and about 2.9% w/w, between about 2.2% w/w and about 2.8% w/w, between about 2.2% w/w and about 2.7% w/w, between about 2.2% w/w and about 2.6% w/w, between about 2.2% w/w and about 2.5% w/w, between about 2.3% w/w and about 2.9% w/w, between about 2.3% w/w and about 2.8% w/w, between about 2.3% w/w and about 2.7% w/w, between about 2.3% w/w and about 2.6% w/w, or between about 2.3% w/w and about 2.5% w/w (e.g., about 2.4% w/w).

(A8) In one embodiment, the formulations provided herein comprise Compound I-A, or a pharmaceutically acceptable salt thereof (e.g., Compound II-A), in an amount between about 0.5% w/w and about 1.5% w/w, between about 0.5% w/w and about 1.4% w/w, between about 0.5% w/w and about 1.3% w/w, between about 0.5% w/w and about 1.2% w/w, between about 0.5% w/w and about 1.1% w/w, between about 0.6% w/w and about 1.5% w/w, between about 0.6% w/w and about 1.4% w/w, between about 0.6% w/w and about 1.3% w/w, between about 0.6% w/w and about 1.2% w/w, between about 0.6% w/w and about 1.1% w/w, between about 0.7% w/w and about 1.5% w/w, between about 0.7% w/w and about 1.4% w/w, between about 0.7% w/w and about 1.3% w/w, between about 0.7% w/w and about 1.2% w/w, between about 0.7% w/w and about 1.1% w/w, between about 0.8% w/w and about 1.5% w/w, between about 0.8% w/w and about 1.4% w/w, between about 0.8% w/w and about 1.3% w/w, between about 0.8% w/w and about 1.2% w/w, between about 0.8% w/w and about 1.1% w/w, between about 0.9% w/w and about 1.5% w/w, between about 0.9% w/w and about 1.4% w/w, between about 0.9% w/w and about 1.3% w/w, between about 0.9% w/w and about 1.2% w/w, or between about 0.9% w/w and about 1.1% w/w (e.g., about 1.0% w/w).

(A9) In one embodiment, the formulations provided herein comprise Compound I-A, or a pharmaceutically acceptable salt thereof (e.g., Compound II-A), in an amount of greater than about 0.5% w/w, greater than about 0.6% w/w, greater than about 0.7% w/w, greater than about 0.8% w/w, greater than about 0.9% w/w, greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2.0% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3.0% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w.

(A10) In one embodiment, the formulations provided herein comprise Compound I-A in concentrations as provided in any one in (A1)-(A9).]

(A11) In another embodiment, the formulations provided herein comprise Compound II-A, in concentrations as provided in any one in (A1)-(A9).

The formulation of the present application may be in the form of an aqueous solution comprising an aqueous vehicle. The aqueous vehicle component of the ophthalmic formulation may comprise water and at least one ophthalmically acceptable excipient. Particularly, the aqueous vehicle comprises a solution of the one or more ophthalmically acceptable excipients in water.

Suitable ophthalmically acceptable excipients include those selected from the group consisting of a solubility enhancing agent, chelating agent, preservative, tonicity agent, viscosity/suspending agent, buffering agent, and pH modifying agent, and a mixture thereof. Particularly, the ophthalmically acceptable excipient is selected from the group consisting of a solubility enhancing agent, chelating agent, preservative, tonicity agent, viscosity/suspending agent, and pH modifying agent, and a mixture thereof.

Any suitable ophthalmically acceptable solubility enhancing agent can be used. Examples of solubility enhancing agents include cyclodextrin, such as hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof. In one embodiment, a solubility enhancing agent includes β-cyclodextrin sulfobutyl ether, hydroxypropyl-β-cyclodextrin, sulphated β-cyclodextrin (S-β-CD), and maltosyl-β-cyclodextrin, and mixtures thereof. In one embodiment, a solubility enhancing agent includes β-cyclodextrin sulfobutyl ether, and hydroxypropyl-β-cyclodextrin, and mixtures thereof. A solubility enhancing agent may be added to the formulations of the present application as provided herein.

(B1) In one embodiment, the formulations provided herein comprise a solubility enhancing agent (e.g., cyclodextrin (e.g., HPβCD)) in an amount of about 0.5% w/w to about 25% w/w, about 0.5% w/w to about 23% w/w, about 0.5% w/w to about 20% w/w, about 1.0% w/w to about 18% w/w, about 1.5% w/w to about 16% w/w, about 2.0% w/w to about 15.5% w/w, about 2.5% w/w to about 15% w/w, about 3.0% w/w to about 15% w/w, about 1.5% w/w to about 10% w/w, about 2.0% w/w to about 8.0% w/w, about 2.0% w/w to about 6.0% w/w, about 2.5% w/w to about 6.0% w/w, about 2.5% w/w to about 3.5% w/w, about 7.0% w/w to about 8.0% w/w, or about 14% w/w to about 15% w/w.

(B2) In another embodiment, the formulations provided herein comprise a solubility enhancing agent (e.g., cyclodextrin (e.g., HPβCD)) in an amount of about 0.5% w/w to about 1.0% w/w, about 1.0% w/w to about 1.5% w/w, about 1.5% w/w to about 2.0% w/w, about 2.0% w/w to about 2.5% w/w, about 2.5% w/w to about 3.0% w/w, about 3.0% w/w to about 3.5% w/w, about 3.5% w/w to about 4.0% w/w, about 4.0% w/w to about 4.5% w/w, about 4.5% w/w to about 5.0% w/w, about 5.0% w/w to about 5.5% w/w, about 5.5% w/w to about 6.0% w/w, about 6.0% w/w to about 6.5% w/w, about 6.5% w/w to about 7.0% w/w, about 7.0% w/w to about 7.5% w/w, about 7.5% w/w to about 8.0% w/w, about 8.0% w/w to about 8.5% w/w, about 8.5% w/w to about 9.0% w/w, about 9.0% w/w to about 9.5% w/w, about 9.5% w/w to about 10.0% w/w, about 10.0% w/w to about 10.5% w/w, about 10.5% w/w to about 11.0% w/w, about 11.0% w/w to about 11.5% w/w, about 11.5% w/w to about 12.0% w/w, about 12.0% w/w to about 12.5% w/w, about 12.5% w/w to about 13.0% w/w, about 13.0% w/w to about 14.0% w/w, about 14.0% w/w to about 15.0% w/w, about 15.0% w/w to about 16.0% w/w, about 16.0% w/w to about 17.0% w/w, or about 17.0% w/w to about 18.0% w/w, about 18.0% w/w to about 19.0% w/w, about 19.0% w/w to about 20.0% w/w, about 20.0% w/w to about 21.0% w/w, about 21.0% w/w to about 22.0% w/w, about 22.0% w/w to about 23.0% w/w, about 23.0% w/w to about 24.0% w/w, about 24.0% w/w to about 25.0% w/w, or any combination thereof (e.g., about 2.5% w/w to about 3.5% w/w, about 7.0% w/w to about 8.0% w/w, about 14.0% w/w to about 15.0% w/w, or about 23.0% w/w to about 24.0% w/w, etc.).

(B3) In a further embodiment, the formulations provided herein comprise a solubility enhancing agent (e.g., cyclodextrin (e.g., HPβCD)) in an amount of greater than about 0.5% w/w, greater than about 1.0% w/w, greater than about 1.5% w/w, greater than about 2.0% w/w, greater than about 2.5% w/w, greater than about 3.0% w/w, greater than about 3.5% w/w, greater than about 4.0% w/w, greater than about 4.1% w/w, greater than about 4.2% w/w, greater than about 4.3% w/w, greater than about 4.4% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, greater than about 20.0% w/w, greater than about 21.0% w/w, greater than about 22.0% w/w, greater than about 23.0% w/w, greater than about 24.0% w/w, or greater than about 25.0% w/w.

(B4) In one embodiment, the formulations provided herein comprise a solubility enhancing agent (e.g., cyclodextrin (e.g., HPβCD)) in an amount between about 22.7% w/w and about 23.7% w/w, between about 22.7% and about 23.6% w/w, between about 22.7% and about 23.5% w/w, between about 22.7% and about 23.4% w/w, between about 22.7% and about 23.3% w/w, between about 22.8% and about 23.7% w/w, between about 22.8% and about 23.6% w/w, between about 22.8% and about 23.5% w/w, between about 22.8% and about 23.4% w/w, between about 22.8% and about 23.3% w/w, between about 22.9% and about 23.7% w/w, between about 22.9% and about 23.6% w/w, between about 22.9% and about 23.5% w/w, between about 22.9% and about 23.4% w/w, between about 22.9% and about 23.3% w/w, between about 23.0% and about 23.7% w/w, between about 23.0% and about 23.6% w/w, between about 23.0% and about 23.5% w/w, between about 23.0% and about 23.4% w/w, between about 23.0% and about 23.3% w/w, between about 23.1% and about 23.7% w/w, between about 23.1% and about 23.6% w/w, between about 23.1% and about 23.5% w/w, between about 23.1% and about 23.4% w/w, between about 23.1% and about 23.3% w/w, between about 23.2% and about 23.7% w/w, between about 23.2% and about 23.6% w/w, between about 23.2% and about 23.5% w/w, between about 23.2% and about 23.4% w/w, or between about 23.2% and about 23.3% w/w (e.g., about 23.25% w/w).

(B5) In one embodiment, the formulations provided herein comprise a solubility enhancing agent (e.g., cyclodextrin (e.g., HPβCD)) in an amount between about 14.1% w/w and about 15.1% w/w, between about 14.1% w/w and about 15.0% w/w, between about 14.1% w/w and about 14.9% w/w, between about 14.1% w/w and about 14.8% w/w, between about 14.1% w/w and about 14.7% w/w, between about 14.2% w/w and about 15.1% w/w, between about 14.2% w/w and about 15.0% w/w, between about 14.2% w/w and about 14.9% w/w, between about 14.2% w/w and about 14.8% w/w, between about 14.2% w/w and about 14.7% w/w, between about 14.3% w/w and about 15.1% w/w, between about 14.3% w/w and about 15.0% w/w, between about 14.3% w/w and about 14.9% w/w, between about 14.3% w/w and about 14.8% w/w, between about 14.3% w/w and about 14.7% w/w, between about 14.4% w/w and about 15.1% w/w, between about 14.4% w/w and about 15.0% w/w, between about 14.4% w/w and about 14.9% w/w, between about 14.4% w/w and about 14.8% w/w, between about 14.4% w/w and about 14.7% w/w, between about 14.5% w/w and about 15.1% w/w, between about 14.5% w/w and about 15.0% w/w, between about 14.5% w/w and about 14.9% w/w, between about 14.5% w/w and about 14.8% w/w, or between about 14.5% w/w and about 14.7% w/w (e.g., about 14.6% w/w).

(B6) In one embodiment, the formulations provided herein comprise a solubility enhancing agent (e.g., cyclodextrin (e.g., HPβCD)) in an amount between about 7.0% w/w and about 8.0% w/w, between about 7.0% w/w and about 7.9% w/w, between about 7.0% w/w and about 7.8% w/w, between about 7.0% w/w and about 7.7% w/w, between about 7.0% w/w and about 7.6% w/w, between about 7.1% w/w and about 8.0% w/w, between about 7.1% w/w and about 7.9% w/w, between about 7.1% w/w and about 7.8% w/w, between about 7.1% w/w and about 7.7% w/w, between about 7.1% w/w and about 7.6% w/w, between about 7.2% w/w and about 8.0% w/w, between about 7.2% w/w and about 7.9% w/w, between about 7.2% w/w and about 7.8% w/w, between about 7.2% w/w and about 7.7% w/w, between about 7.2% w/w and about 7.6% w/w, between about 7.3% w/w and about 8.0% w/w, between about 7.3% w/w and about 7.9% w/w, between about 7.3% w/w and about 7.8% w/w, between about 7.3% w/w and about 7.7% w/w, between about 7.3% w/w and about 7.6% w/w, between about 7.4% w/w and about 8.0% w/w, between about 7.4% w/w and about 7.9% w/w, between about 7.4% w/w and about 7.8% w/w, between about 7.4% w/w and about 7.7% w/w, or between about 7.4% w/w and about 7.6% w/w (e.g., about 7.5% w/w).

(B7) In one embodiment, the formulations provided herein comprise a solubility enhancing agent (e.g., cyclodextrin (e.g., HPβCD)) in an amount between about 2.6% w/w and about 3.6% w/w, between about 2.6% w/w and about 3.5% w/w, between about 2.6% w/w and about 3.4% w/w, between about 2.6% w/w and about 3.3% w/w, between about 2.6% w/w and about 3.2% w/w, between about 2.7% w/w and about 3.6% w/w, between about 2.7% w/w and about 3.5% w/w, between about 2.7% w/w and about 3.4% w/w, between about 2.7% w/w and about 3.3% w/w, between about 2.7% w/w and about 3.2% w/w, between about 2.8% w/w and about 3.6% w/w, between about 2.8% w/w and about 3.5% w/w, between about 2.8% w/w and about 3.4% w/w, between about 2.8% w/w and about 3.3% w/w, between about 2.8% w/w and about 3.2% w/w, between about 2.9% w/w and about 3.6% w/w, between about 2.9% w/w and about 3.5% w/w, between about 2.9% w/w and about 3.4% w/w, between about 2.9% w/w and about 3.3% w/w, between about 2.9% w/w and about 3.2% w/w, between about 3.0% w/w and about 3.6% w/w, between about 3.0% w/w and about 3.5% w/w, between about 3.0% w/w and about 3.4% w/w, between about 3.0% w/w and about 3.3% w/w, or between about 3.0% w/w and about 3.2% w/w (e.g., about 3.1% w/w).

(B8) In one embodiment, the formulations provided herein comprise a solubility enhancing agent (e.g., cyclodextrin (e.g., HPβCD)), wherein the weight ratio between the solubility enhancing agent (e.g., cyclodextrin (e.g., HPβCD)) and Compound I-A, or the pharmaceutically acceptable salt thereof (e.g., Compound II-A) is greater than about 1:1, greater than about 1.1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

(B9) In one embodiment, the formulations provided herein comprise a solubility enhancing agent (e.g., cyclodextrin (e.g., HPβCD)), wherein the weight ratio between the solubility enhancing agent (e.g., cyclodextrin (e.g., HPβCD)) and Compound I-A, or the pharmaceutically acceptable salt thereof (e.g., Compound II-A) is from about 1:1 to about 6:1, from about 1:1 to about 5.5:1, from about 1:1 to about 5:1, from about 1:1 to about 4.5:1, from about 1:1 to about 4:1, from about 1:1 to about 3.9:1, from about 1:1 to about 3.8:1, from about 1:1 to about 3.7:1, from about 1:1 to about 3.6:1, from about 1:1 to about 3.5:1, from about 1:1 to about 3.4:1, from about 1:1 to about 3.3:1, from about 1:1 to about 3.2:1, from about 1:1 to about 3.1:1, from about 1.2:1 to about 6:1, from about 1.2:1 to about 5:1, from about 1.2:1 to about 4.5:1, from about 1.2:1 to about 4:1, from about 1.2:1 to about 3.9:1, from about 1.2:1 to about 3.8:1, from about 1.2:1 to about 3.7:1, from about 1.2:1 to about 3.6:1, from about 1.2:1 to about 3.5:1, from about 1.2:1 to about 3.4:1, from about 1.2:1 to about 3.3:1, from about 1.2:1 to about 3.2:1, from about 1.2:1 to about 3.1:1, from about 1.25:1 to about 6:1, from about 1.25:1 to about 5:1, from about 1.25:1 to about 4.5:1, from about 1.25:1 to about 4:1, from about 1.25:1 to about 3.9:1, from about 1.25:1 to about 3.8:1, from about 1.25:1 to about 3.7:1, from about 1.25:1 to about 3.6:1, from about 1.25:1 to about 3.5:1, from about 1.25:1 to about 3.4:1, from about 1.25:1 to about 3.3:1, from about 1.25:1 to about 3.2:1, from about 1.25:1 to about 3.1:1, from about 1.3:1 to about 6:1, from about 1.3:1 to about 5:1, from about 1.3:1 to about 4.5:1, from about 1.3:1 to about 4:1, from about 1.3:1 to about 3.9:1, from about 1.3:1 to about 3.8:1, from about 1.3:1 to about 3.7:1, from about 1.3:1 to about 3.6:1, from about 1.3:1 to about 3.5:1, from about 1.3:1 to about 3.4:1, from about 1.3:1 to about 3.3:1, from about 1.3:1 to about 3.2:1, from about 1.3:1 to about 3.1:1, from about 1.4:1 to about 6:1, from about 1.4:1 to about 5:1, from about 1.4:1 to about 4.5:1, from about 1.4:1 to about 4:1, from about 1.4:1 to about 3.9:1, from about 1.4:1 to about 3.8:1, from about 1.4:1 to about 3.7:1, from about 1.4:1 to about 3.6:1, from about 1.4:1 to about 3.5:1, from about 1.4:1 to about 3.4:1, from about 1.4:1 to about 3.3:1, from about 1.4:1 to about 3.2:1, from about 1.4:1 to about 3.1:1, from about 1.5:1 to about 6:1, from about 1.5:1 to about 5:1, from about 1.5:1 to about 4.5:1, from about 1.5:1 to about 4:1, from about 1.5:1 to about 3.9:1, from about 1.5:1 to about 3.8:1, from about 1.5:1 to about 3.7:1, from about 1.5:1 to about 3.6:1, from about 1.5:1 to about 3.5:1, from about 1.5:1 to about 3.4:1, from about 1.5:1 to about 3.3:1, from about 1.5:1 to about 3.2:1, from about 1.5:1 to about 3.1:1, from about 1.6:1 to about 6:1, from about 1.6:1 to about 5:1, from about 1.6:1 to about 4.5:1, from about 1.6:1 to about 4:1, from about 1.6:1 to about 3.9:1, from about 1.6:1 to about 3.8:1, from about 1.6:1 to about 3.7:1, from about 1.6:1 to about 3.6:1, from about 1.6:1 to about 3.5:1, from about 1.6:1 to about 3.4:1, from about 1.6:1 to about 3.3:1, from about 1.6:1 to about 3.2:1, from about 1.6:1 to about 3.1:1, from about 1.7:1 to about 6:1, from about 1.7:1 to about 5:1, from about 1.7:1 to about 4.5:1, from about 1.7:1 to about 4:1, from about 1.7:1 to about 3.9:1, from about 1.7:1 to about 3.8:1, from about 1.7:1 to about 3.7:1, from about 1.7:1 to about 3.6:1, from about 1.7:1 to about 3.5:1, from about 1.7:1 to about 3.4:1, from about 1.7:1 to about 3.3:1, from about 1.7:1 to about 3.2:1, from about 1.7:1 to about 3.1:1, from about 1.8:1 to about 6:1, from about 1.8:1 to about 5:1, from about 1.8:1 to about 4.5:1, from about 1.8:1 to about 4:1, from about 1.8:1 to about 3.9:1, from about 1.8:1 to about 3.8:1, from about 1.8:1 to about 3.7:1, from about 1.8:1 to about 3.6:1, from about 1.8:1 to about 3.5:1, from about 1.8:1 to about 3.4:1, from about 1.8:1 to about 3.3:1, from about 1.8:1 to about 3.2:1, from about 1.8:1 to about 3.1:1, from about 1.9:1 to about 6:1, from about 1.9:1 to about 5:1, from about 1.9:1 to about 4.5:1, from about 1.9:1 to about 4:1, from about 1.9:1 to about 3.9:1, from about 1.9:1 to about 3.8:1, from about 1.9:1 to about 3.7:1, from about 1.9:1 to about 3.6:1, from about 1.9:1 to about 3.5:1, from about 1.9:1 to about 3.4:1, from about 1.9:1 to about 3.3:1, from about 1.9:1 to about 3.2:1, from about 1.9:1 to about 3.1:1, from about 2:1 to about 6:1, from about 2:1 to about 5:1, from about 2:1 to about 4.5:1, from about 2:1 to about 4:1, from about 2:1 to about 3.9:1, from about 2:1 to about 3.8:1, from about 2:1 to about 3.7:1, from about 2:1 to about 3.6:1, from about 2:1 to about 3.5:1, from about 2:1 to about 3.4:1, from about 2:1 to about 3.3:1, from about 2:1 to about 3.2:1, from about 2:1 to about 3.1:1, from about 2.1:1 to about 6:1, from about 2.1:1 to about 5:1, from about 2.1:1 to about 4.5:1, from about 2.1:1 to about 4:1, from about 2.1:1 to about 3.9:1, from about 2.1:1 to about 3.8:1, from about 2.1:1 to about 3.7:1, from about 2.1:1 to about 3.6:1, from about 2.1:1 to about 3.5:1, from about 2.1:1 to about 3.4:1, from about 2.1:1 to about 3.3:1, from about 2.1:1 to about 3.2:1, from about 2.1:1 to about 3.1:1, from about 2.2:1 to about 6:1, from about 2.2:1 to about 5:1, from about 2.2:1 to about 4.5:1, from about 2.2:1 to about 4:1, from about 2.2:1 to about 3.9:1, from about 2.2:1 to about 3.8:1, from about 2.2:1 to about 3.7:1, from about 2.2:1 to about 3.6:1, from about 2.2:1 to about 3.5:1, from about 2.2:1 to about 3.4:1, from about 2.2:1 to about 3.3:1, from about 2.2:1 to about 3.2:1, from about 2.2:1 to about 3.1:1, from about 2.3:1 to about 6:1, from about 2.3:1 to about 5:1, from about 2.3:1 to about 4.5:1, from about 2.3:1 to about 4:1, from about 2.3:1 to about 3.9:1, from about 2.3:1 to about 3.8:1, from about 2.3:1 to about 3.7:1, from about 2.3:1 to about 3.6:1, from about 2.3:1 to about 3.5:1, from about 2.3:1 to about 3.4:1, from about 2.3:1 to about 3.3:1, from about 2.3:1 to about 3.2:1, from about 2.3:1 to about 3.1:1, from about 2.4:1 to about 6:1, from about 2.4:1 to about 5:1, from about 2.4:1 to about 4.5:1, from about 2.4:1 to about 4:1, from about 2.4:1 to about 3.9:1, from about 2.4:1 to about 3.8:1, from about 2.4:1 to about 3.7:1, from about 2.4:1 to about 3.6:1, from about 2.4:1 to about 3.5:1, from about 2.4:1 to about 3.4:1, from about 2.4:1 to about 3.3:1, from about 2.4:1 to about 3.2:1, from about 2.4:1 to about 3.1:1, from about 2.5:1 to about 6:1, from about 2.5:1 to about 5:1, from about 2.5:1 to about 4.5:1, from about 2.5:1 to about 4:1, from about 2.5:1 to about 3.9:1, from about 2.5:1 to about 3.8:1, from about 2.5:1 to about 3.7:1, from about 2.5:1 to about 3.6:1, from about 2.5:1 to about 3.5:1, from about 2.5:1 to about 3.4:1, from about 2.5:1 to about 3.3:1, from about 2.5:1 to about 3.2:1, from about 2.5:1 to about 3.1:1, from about 2.6:1 to about 6:1, from about 2.6:1 to about 5:1, from about 2.6:1 to about 4.5:1, from about 2.6:1 to about 4:1, from about 2.6:1 to about 3.9:1, from about 2.6:1 to about 3.8:1, from about 2.6:1 to about 3.7:1, from about 2.6:1 to about 3.6:1, from about 2.6:1 to about 3.5:1, from about 2.6:1 to about 3.4:1, from about 2.6:1 to about 3.3:1, from about 2.6:1 to about 3.2:1, from about 2.6:1 to about 3.1:1, from about 2.7:1 to about 6:1, from about 2.7:1 to about 5:1, from about 2.7:1 to about 4.5:1, from about 2.7:1 to about 4:1, from about 2.7:1 to about 3.9:1, from about 2.7:1 to about 3.8:1, from about 2.7:1 to about 3.7:1, from about 2.7:1 to about 3.6:1, from about 2.7:1 to about 3.5:1, from about 2.7:1 to about 3.4:1, from about 2.7:1 to about 3.3:1, from about 2.7:1 to about 3.2:1, from about 2.7:1 to about 3.1:1, from about 2.8:1 to about 6:1, from about 2.8:1 to about 5:1, from about 2.8:1 to about 4.5:1, from about 2.8:1 to about 4:1, from about 2.8:1 to about 3.9:1, from about 2.8:1 to about 3.8:1, from about 2.8:1 to about 3.7:1, from about 2.8:1 to about 3.6:1, from about 2.8:1 to about 3.5:1, from about 2.8:1 to about 3.4:1, from about 2.8:1 to about 3.3:1, from about 2.8:1 to about 3.2:1, from about 2.8:1 to about 3.1:1, or from about 2.9:1 to about 6:1, from about 2.9:1 to about 5:1, from about 2.9:1 to about 4.5:1, from about 2.9:1 to about 4:1, from about 2.9:1 to about 3.9:1, from about 2.9:1 to about 3.8:1, from about 2.9:1 to about 3.7:1, from about 2.9:1 to about 3.6:1, from about 2.9:1 to about 3.5:1, from about 2.9:1 to about 3.4:1, from about 2.9:1 to about 3.3:1, from about 2.9:1 to about 3.2:1, or from about 2.9:1 to about 3.1:1 (e.g., about 3:1).

(B10) In one embodiment, the formulations provided herein comprise a solubility enhancing agent (e.g., cyclodextrin (e.g., HPβCD)), wherein the molar ratio between the solubility enhancing agent (e.g., cyclodextrin (e.g., HPβCD)) and Compound I-A, or the pharmaceutically acceptable salt thereof (e.g., Compound II-A) is greater than about 0.8:1, greater than about 0.9:1, greater than about 1.0:1, greater than about 1.1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2.0:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3.0:1, greater than about 3.5:1, greater than about 4.0:1, greater than about 4.5:1, greater than about 5.0:1, greater than about 5.5:1, greater than about 6.0:1, greater than about 6.5:1, greater than about 7.0:1, greater than about 7.5:1, greater than about 8.0:1, greater than about 8.5:1, greater than about 9.0:1, greater than about 9.5:1, or greater than about 10.0:1.

(B11) In one embodiment, the formulations provided herein comprise a solubility enhancing agent (e.g., cyclodextrin (e.g., HPβCD)), wherein the molar ratio between the solubility enhancing agent (e.g., cyclodextrin (e.g., HPβCD)) and Compound I-A, or the pharmaceutically acceptable salt thereof (e.g., Compound II-A) is from about 0.8:1 to about 5:1, from about 0.8:1 to about 4.5:1, from about 0.8:1 to about 4:1, from about 0.8:1 to about 3.5:1, from about 0.8:1 to about 3:1, from about 0.8:1 to about 2.5:1, from about 0.8:1 to about 2.0:1, from about 0.8:1 to about 1.9:1, from about 0.8:1 to about 1.8:1, from about 0.8:1 to about 1.7:1, from about 0.8:1 to about 1.6:1, from about 0.8:1 to about 1.5:1, from about 0.8:1 to about 1.4:1, from about 0.8:1 to about 1.3:1, from about 0.8:1 to about 1.2:1, from about 0.8:1 to about 1.1:1, from about 0.8:1 to about 1:1, from about 0.9:1 to about 5:1, from about 0.9:1 to about 4.5:1, from about 0.9:1 to about 4:1, from about 0.9:1 to about 3.5:1, from about 0.9:1 to about 3:1, from about 0.9:1 to about 2.5:1, from about 0.9:1 to about 2.0:1, from about 0.9:1 to about 1.9:1, from about 0.9:1 to about 1.8:1, from about 0.9:1 to about 1.7:1, from about 0.9:1 to about 1.6:1, from about 0.9:1 to about 1.5:1, from about 0.9:1 to about 1.4:1, from about 0.9:1 to about 1.3:1, from about 0.9:1 to about 1.2:1, from about 0.9:1 to about 1.1:1, from about 0.9:1 to about 1:1, from about 1:1 to about 5:1, from about 1:1 to about 4.5:1, from about 1:1 to about 4:1, from about 1:1 to about 3.5:1, from about 1:1 to about 3:1, from about 1:1 to about 2.5:1, from about 1:1 to about 2.0:1, from about 1:1 to about 1.9:1, from about 1:1 to about 1.8:1, from about 1:1 to about 1.7:1, from about 1:1 to about 1.6:1, from about 1:1 to about 1.5:1, from about 1:1 to about 1.4:1, from about 1:1 to about 1.3:1, from about 1:1 to about 1.2:1, from about 1:1 to about 1.1:1, from about 1.1:1 to about 5:1, from about 1.1:1 to about 4.5:1, from about 1.1:1 to about 4:1, from about 1.1:1 to about 3.5:1, from about 1.1:1 to about 3:1, from about 1.1:1 to about 2.5:1, from about 1.1:1 to about 2.0:1, from about 1.1:1 to about 1.9:1, from about 1.1:1 to about 1.8:1, from about 1.1:1 to about 1.7:1, from about 1.1:1 to about 1.6:1, from about 1.1:1 to about 1.5:1, from about 1.1:1 to about 1.4:1, from about 1.1:1 to about 1.3:1, from about 1.1:1 to about 1.2:1, from about 1.15:1 to about 1.35:1, from about 1.2:1 to about 1.3:1 (e.g., about 1.25:1), from about 1.2:1 to about 5:1, from about 1.2:1 to about 4.5:1, from about 1.2:1 to about 4:1, from about 1.2:1 to about 3.5:1, from about 1.2:1 to about 3:1, from about 1.2:1 to about 2.5:1, from about 1.2:1 to about 2.0:1, from about 1.2:1 to about 1.9:1, from about 1.2:1 to about 1.8:1, from about 1.2:1 to about 1.7:1, from about 1.2:1 to about 1.6:1, from about 1.2:1 to about 1.5:1, from about 1.2:1 to about 1.4:1, from about 1.2:1 to about 1.3:1, from about 1.3:1 to about 5:1, from about 1.3:1 to about 4.5:1, from about 1.3:1 to about 4:1, from about 1.3:1 to about 3.5:1, from about 1.3:1 to about 3:1, from about 1.3:1 to about 2.5:1, from about 1.3:1 to about 2.0:1, from about 1.3:1 to about 1.9:1, from about 1.3:1 to about 1.8:1, from about 1.3:1 to about 1.7:1, from about 1.3:1 to about 1.6:1, from about 1.3:1 to about 1.5:1, from about 1.3:1 to about 1.4:1, from about 1.4:1 to about 5:1, from about 1.4:1 to about 4.5:1, from about 1.4:1 to about 4:1, from about 1.4:1 to about 3.5:1, from about 1.4:1 to about 3:1, from about 1.4:1 to about 2.5:1, from about 1.4:1 to about 2.0:1, from about 1.4:1 to about 1.9:1, from about 1.4:1 to about 1.8:1, from about 1.4:1 to about 1.7:1, from about 1.4:1 to about 1.6:1, from about 1.4:1 to about 1.5:1, from about 1.5:1 to about 5:1, from about 1.5:1 to about 4.5:1, from about 1.5:1 to about 4:1, from about 1.5:1 to about 3.5:1, from about 1.5:1 to about 3:1, from about 1.5:1 to about 2.5:1, from about 1.5:1 to about 2.0:1, from about 1.5:1 to about 1.9:1, from about 1.5:1 to about 1.8:1, from about 1.5:1 to about 1.7:1, or from about 1.5:1 to about 1.6:1.

(B12) In one embodiment, the solubility enhancing agent present in the formulation according to any one in (B1)-(B11) is a cyclodextrin.

(B13) In a further embodiment, the cyclodextrin according to (B12) is a β-cyclodextrin sulfobutyl ether. In an even further embodiment, the β-cyclodextrin sulfobutyl ether is sulfobutyl ether β-cyclodextrin-7 or sulfobutyl ether β-cyclodextrin-4.

(B14) In yet another embodiment, the cyclodextrin according to (B12) is HPβCD.

Any suitable ophthalmically acceptable chelating agent can be used. Examples of chelating agents include those selected from the group consisting of ethylenediaminetetraacetic acid and metal salts thereof, including disodium edetate, trisodium edetate, and tetrasodium edetate, and mixtures thereof. The chelating agent(s) may be added to the formulations of the present application as provided herein:

(C1) In one embodiment, the chelating agent(s) may be added in an amount of about 0.001% w/w to about 1.0% w/w, about 0.005% w/w to about 0.75% w/w, about 0.01% w/w to about 0.5% w/w, about 0.05% w/w to about 0.2% w/w, or about 0.075% w/w to about 0.15% w/w.

(C2) In another embodiment, the chelating agent(s) may be added in an amount of about 0.001% w/w to about 0.005% w/w, about 0.005% w/w to about 0.01% w/w, about 0.01% w/w to about 0.02% w/w, about 0.02% w/w to about 0.03% w/w, about 0.03% w/w to about 0.04% w/w, about 0.04% w/w to about 0.05% w/w, about 0.05% w/w to about 0.06% w/w, about 0.06% w/w to about 0.07% w/w, about 0.07% w/w to about 0.075% w/w, about 0.075% w/w to about 0.08% w/w, about 0.08% w/w to about 0.085% w/w, about 0.085% w/w to about 0.09% w/w, about 0.09% w/w to about 0.095% w/w, about 0.095% w/w to about 0.1% w/w, about 0.1% w/w to about 0.105% w/w, about 0.105% w/w to about 0.11% w/w, about 0.11% w/w to about 0.115% w/w, about 0.115% w/w to about 0.12% w/w, about 0.12% w/w to about 0.125% w/w, about 0.125% w/w to about 0.13% w/w, about 0.13% w/w to about 0.135% w/w, about 0.135% w/w to about 0.14% w/w, about 0.14% w/w to about 0.145% w/w, about 0.145% w/w to about 0.15% w/w, about 0.15% w/w to about 0.16% w/w, about 0.16% w/w to about 0.17% w/w, about 0.17% w/w to about 0.18% w/w, about 0.18% w/w to about 0.19% w/w, or about 0.19% w/w to about 0.2% w/w, or any combination thereof (e.g., about 0.005% w/w to about 0.2% w/w, about 0.01% w/w to about 0.1% w/w, etc.).

(C3) In a further embodiment, the chelating agent(s) may be added in an amount of about 0.1% w/w.

(C4) In one embodiment, the chelating agent present in the formulation according to (C1)-(C3) is an ethylenediaminetetraacetic acid salt. In a further embodiment, the ethylenediaminetetraacetic acid salt is disodium edetate.

Any suitable ophthalmically acceptable preservatives can be used. Examples of preservatives include those selected from the group consisting of quaternary ammonium salts such as benzalkonium halides (particularly benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, sorbic acid, and mixtures thereof. Particularly, the preservative is a quaternary ammonium salt such as benzalkonium halides (particularly benzalkonium chloride), benzoxonium halides (particularly benzoxonium chloride), chlorhexidine gluconate, benzethonium chloride, cetyl pyridinium chloride, potassium sorbate, sodium benzoate, ethyl p-hydroxybenzoate, butyl p-hydroxybenzoate, or propylaminopropyl biguanide, or mixtures thereof.

The preservative(s) may be added to the formulations of the present application as provided herein:

(D1) In one embodiment, the preservative(s) may be added in an amount between about 0.001% w/w and about 0.5% w/w, between about 0.0025% w/w and about 0.1% w/w, between about 0.005% w/w and about 0.05% w/w, and between about 0.01% w/w and about 0.025% w/w.

(D2) In another embodiment, the preservative(s) may be added in an amount between about 0.001% w/w and about 0.003% w/w, between about 0.003% w/w and about 0.005% w/w, between about 0.005% w/w and about 0.0075% w/w, between about 0.0075% w/w and about 0.01% w/w, between about 0.01% w/w and about 0.015% w/w, between about 0.015% w/w and about 0.02% w/w, between about 0.02% w/w and about 0.025% w/w, between about 0.025% w/w and about 0.03% w/w, between about 0.03% w/w and about 0.035% w/w, between about 0.035% w/w and about 0.04% w/w, between about 0.04% w/w and about 0.045% w/w, between about 0.045% w/w and about 0.05% w/w, between about 0.05% w/w and about 0.055% w/w, between about 0.055% w/w and about 0.06% w/w, between about 0.065% w/w and about 0.07% w/w, between about 0.07% w/w and about 0.075% w/w, between about 0.075% w/w and about 0.08% w/w, between about 0.08% w/w and about 0.085% w/w, between about 0.085% w/w and about 0.09% w/w, between about 0.09% w/w and about 0.095% w/w, between about 0.095% w/w and about 0.1% w/w, between about 0.1% w/w and about 0.125% w/w, between about 0.125% w/w and about 0.15% w/w, between about 0.15% w/w and about 0.175% w/w, between about 0.175% w/w and about 0.2% w/w, between about 0.2% w/w and about 0.3% w/w, between about 0.3% w/w and about 0.4% w/w, or between about 0.4% w/w and about 0.5% w/w, or any combination thereof (e.g., between about 0.005% w/w and about 0.02% w/w, between about 0.01% w/w and about 0.02% w/w, etc.).

(D3) In a further embodiment, the preservative(s) may be added in an amount of about 0.005% w/w, about 0.01% w/w, about 0.015% w/w, or about 0.02% w/w.

(D4) In one embodiment, the preservative present in the formulation according to (D1)-(D3) is a benzalkonium halide. In a further embodiment, the benzalkonium halide is BAC.

(D5) In another embodiment, the preservative present in the formulation according to (D1)-(D3) is a benzoxonium halide. In yet another embodiment, the benzoxonium halide is benzoxonium chloride.

Any suitable ophthalmically acceptable tonicity agent can be used to adjust the tonicity (osmotic pressure) in order to achieve an ophthalmically compatible formulation. The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. Particularly, the tonicity agent is selected from the group consisting of glycerin, mannitol, potassium chloride, and sodium chloride. More particularly mannitol, or sodium chloride, or a mixture thereof is employed. More particularly, sodium chloride is used. The tonicity agent(s) may be used in an amount of about 0.05% w/w to about 8% w/w.

When a mixture of mannitol and sodium chloride is used as tonicity agents, the weight ratio of mannitol:sodium chloride is about 4:1 to about 15:1, more particularly about 6:1 to about 14:1, or about 8:1 to about 14:1

Any suitable ophthalmically acceptable viscosity/suspending agent can be used. Suitable viscosity/suspending agents include those selected from the group consisting of cellulose derivatives, such as methyl cellulose, ethyl cellulose, hydroxyethylcellulose, carboxymethyl cellulose, hydroxypropylmethyl cellulose, and cross-linked acrylic acid polymers (carbomers), such as polymers of acrylic acid cross-linked with polyalkenyl ethers or divinyl glycol (polyvinylpolypyrrolidone, Carbopols—such as Carbopol 934, Carbopol 934P, Carbopol 971, Carbopol 974 and Carbopol 974P), polyethylene glycols (such as polyethylene glycol 300, polyethylene glycol 400, polyethylene glycol 6000), water-soluble polymers such as polyvinylpyrrolidone, and a mixture thereof. The viscosity/suspending agent(s) may be added to the formulations of the present application as provided herein:

(E1) In one embodiment, the viscosity/suspending agent(s) may be added in an amount between about 0.1% w/w and about 20% w/w, between about 0.25% w/w and about 15% w/w, between about 0.5% w/w and about 10% w/w, between about 0.5% w/w and about 5% w/w, or between about 0.5% w/w and about 3% w/w.

(E2) In another embodiment, the viscosity/suspending agent(s) may be added in an amount between about 0.1% w/w and about 0.3% w/w, between about 0.3% w/w and about 0.5% w/w, between about 0.5% w/w and about 0.75% w/w, between about 0.75% w/w and about 1% w/w, between about 1% w/w and about 1.25% w/w, between about 1.25% w/w and about 1.5% w/w, between about 1.5% w/w and about 1.75% w/w, between about 1.75% w/w and about 2% w/w, between about 2% w/w and about 2.25% w/w, between about 2.25% w/w and about 2.5% w/w, between about 2.5% w/w and about 2.75% w/w, between about 2.75% w/w and about 3% w/w, between about 3% w/w and about 3.5% w/w, between about 3.5% w/w and about 4% w/w, between about 4% w/w and about 4.5% w/w, between about 4.5% w/w and about 5% w/w, between about 5% w/w and about 6% w/w, between about 6% w/w and about 7% w/w, between about 7% w/w and about 8% w/w, between about 8% w/w and about 9% w/w, between about 9% w/w and about 10% w/w, between about 10% w/w and about 12.5% w/w, between about 12.5% w/w and about 15% w/w, between about 15% w/w and about 17.5% w/w, or between about 17.5% w/w and about 20% w/w, or any combination thereof (e.g., between about 0.1% w/w and about 10% w/w, between about 0.5% w/w and about 2% w/w, etc.).

(E3) In another embodiment, the viscosity/suspending agent(s) may be added in an amount of about 1% w/w or about 2% w/w.

(E4) In one embodiment, the viscosity/suspending agent present in the formulation according to (E1)-(E3) is a polyethylene glycol. In a further embodiment, the polyethylene glycol is polyethylene glycol 6000 (PEG 6000).

(E5) In one embodiment, the viscosity/suspending agent present in the formulation according to (E1)-(E3) is a water-soluble polymer. In a further embodiment, the water-soluble polymer according is polyvinylpyrrolidone.

Any suitable ophthalmically acceptable buffering agent can be used to stabilize the pH of the formulation. When used, the buffering agent may be selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof. Particularly, the buffering agent may be a borate buffer (such as boric acid, or salts thereof including disodium tetraborate) or a citrate buffer (such as citric acid, or salts thereof including sodium citrate). The buffering agent(s) may be added to the formulations of the present application as provided herein:

(F1) In one embodiment, the buffering agent(s) may be added in an amount between about 0.01% w/w and about 10% w/w, between about 0.1% w/w and about 5% w/w, between about 0.5% w/w and about 3% w/w, or between about 0.5% w/w and about 2% w/w.

(F2) In another embodiment, the buffering agent(s) may be added in an amount between about 0.01% w/w and about 0.05% w/w, between about 0.05% w/w and about 0.1% w/w, between about 0.1% w/w and about 0.2% w/w, between about 0.2% w/w and about 0.3% w/w, between about 0.3% w/w and about 0.4% w/w, between about 0.4% w/w and about 0.5% w/w, between about 0.5% w/w and about 0.6% w/w, between about 0.6% w/w and about 0.7% w/w, between about 0.7% w/w and about 0.8% w/w, between about 0.8% w/w and about 0.9% w/w, between about 0.9% w/w and about 1% w/w, between about 1% w/w and about 1.5% w/w, between about 1.5% w/w and about 2% w/w, between about 2% w/w and about 2.5% w/w, between about 2.5% w/w and about 3% w/w, between about 3% w/w and about 3.5% w/w, between about 3.5% w/w and about 4% w/w, between about 4% w/w and about 4.5% w/w, between about 4.5% w/w and about 5% w/w, between about 5% w/w and about 6% w/w, between about 6% w/w and about 7% w/w, between about 7% w/w and about 8% w/w, between about 8% w/w and about 9% w/w, or between about 9% w/w and about 10% w/w, or any combination thereof (e.g., between about 0.1% w/w and about 5% w/w, between about 0.5% w/w and about 2% w/w, etc.).

(F3) In another embodiment, the buffering agent(s) may be added in an amount of about 1% w/w.

(F4) In one embodiment, the buffering agent present in the formulation according to (F1)-(F3) comprises boric acid buffer.

(F5) In another embodiment, the buffering agent present in the formulation according to (F1)-(F3) comprises citrate buffer.

In order to adjust the formulation to an ophthalmically acceptable pH (typically a pH range of about 5.0 to about 9.0, about 5.5 to about 8.5, about 6.0 to about 8.5, about 7.0 to about 8.5, about 7.5 to about 8.3, about 7.5 to about 8.3, or about 7.5 to about 8.0), the formulation may contain a pH modifying agent. In one embodiment, the pH of the formulation is adjusted to a value of about 8. The pH modifying agent is typically a mineral acid or metal hydroxide base, selected from the group of potassium hydroxide, sodium hydroxide, hydrochloric acid, and boric acid, and mixtures thereof, and particularly sodium hydroxide and/or hydrochloride acid. These acidic and/or basic pH modifying agents are added to adjust the formulation to the target ophthalmically acceptable pH range. Hence it may not be necessary to use both acid and base—depending on the formulation, the addition of one of the acid or base may be sufficient to bring the mixture to the desired pH range. The pH modifying agent(s) may be present in any amount necessary to adjust the pH of the formulation to an ophthalmically acceptable pH, such as the pH described above. The pH modifying agent(s) may be added to the formulations of the present application as provided herein.

The ophthalmic formulation for topical administration to the eye may further comprise a wetting agent. In any embodiment of the present application the wetting agent is preferably a non-ionic wetting agent. More preferably, the wetting agent is water soluble or swellable. Most preferably the wetting agent is water soluble. “Water soluble” is to be understood in the manner used in standard texts such as the “Handbook of Pharmaceutical Excipients” (Raymond C Rowe, Paul J Sheskey and Sian C Owen, Fifth Edition, Pharmaceutical Press and American Pharmacists Association 2006). Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Specific examples of suitable wetting agents include those selected from the group consisting of: polyoxyethylene-polyoxypropylene block copolymers (poloxamers) such as: polyoxyethylene (160) polyoxypropylene (30) glycol [Pluronic® F68], polyoxyethylene (42) polyoxypropylene (67) glycol [Pluronic® P123], polyoxyethylene (54) polyoxypropylene (39) glycol [Pluronic® P85], polyoxyethylene (196) polyoxypropylene (67) glycol [Poloxamer 407, Pluronic® F127], polyoxyethylene (20) polyoxypropylene (20) glycol [Pluronic® L44], polyoxyethylenated sorbitan esters (polysorbates) such as poly(oxyethylene)sorbitan monopalmitate (polysorbate 40), poly(oxyethylene)sorbitan monostearate (polysorbate 60), poly(oxyethylene)sorbitan tristearate (polysorbate 65), poly(oxyethylene) sorbitan monooleate (polysorbate 80), poly(oxyethylene) sorbitan monolaurate, poly(oxyethylene) sorbitan trioleate, polyethoxylated ethers of castor oils such as polyoxyethylene hydrogenated castor oil 10, polyoxyethylene hydrogenated castor oil 40, polyoxyethylene hydrogenated castor oil 50 and polyoxyethylene hydrogenated castor oil 60, polyoxyl 40 stearate, sucrose fatty esters, and polyoxyethylene fatty esters, and mixtures thereof.

Particularly, the wetting agent is selected from the group consisting of: polyoxyethylene-polyoxypropylene block copolymers (poloxamers) such as: polyoxyethylene (160) polyoxypropylene (30) glycol [Pluronic® F68], polyoxyethylene (42) polyoxypropylene (67) glycol [Pluronic® P123], polyoxyethylene (54) polyoxypropylene (39) glycol [Pluronic® P85], polyoxyethylene (196) polyoxypropylene (67) glycol [Poloxamer 407, Pluronic® F127], and polyoxyethylene (20) polyoxypropylene (20) glycol [Pluronic® L44], polyoxyethylenated sorbitan esters (polysorbates) such as poly(oxyethylene)sorbitan monopalmitate (polysorbate 40), poly(oxyethylene)sorbitan monostearate (polysorbate 60), poly(oxyethylene)sorbitan tristearate (polysorbate 65), poly(oxyethylene) sorbitan monooleate (polysorbate 80), poly(oxyethylene) sorbitan monolaurate, and poly(oxyethylene) sorbitan trioleate, and mixtures thereof.

(G1) In one embodiment, formulations for topical administration to the eye of the present application comprise Compound I-A, or a pharmaceutically acceptable salt or solvate thereof (e.g., Compound II-A), and a solubility enhancing agent.

(G2) In one embodiment, formulations for topical administration to the eye of the present application comprise Compound I-A, or a pharmaceutically acceptable salt or solvate thereof (e.g., Compound II-A), a solubility enhancing agent, and a preservative.

(G3) In one embodiment, formulations for topical administration to the eye of the present application comprise Compound I-A, or a pharmaceutically acceptable salt or solvate thereof (e.g., Compound II-A), a solubility enhancing agent, a preservative, a buffering agent, and a pH modifying agent.

(H) In another embodiment, formulations for topical administration to the eye of the present application (e.g., as described herein, such as in (G1)-(G3)) further comprise a viscosity/suspending agent. In another embodiment, formulations for topical administration to the eye of the present application (e.g., as described herein, such as in (G1)-(G3)) further comprise a chelating agent. In yet another embodiment, formulations for topical administration to the eye of the present application (e.g., as described herein, such as in (G1)-(G3)) further comprise a viscosity/suspending agent and a chelating agent.

(I) In another embodiment, formulations for topical administration to the eye of the present application (e.g., as described herein, such as in (G1)-(G3) and (H)) further comprise a tonicity agent.

(J1) In one embodiment, formulations for topical administration to the eye of the present application comprise Compound I-A, or a pharmaceutically acceptable salt or solvate thereof (e.g., Compound II-A) as set forth in (A1)-(A11). In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J2) In another embodiment, formulations for topical administration to the eye of the present application comprise Compound I-A, or a pharmaceutically acceptable salt or solvate thereof (e.g., Compound II-A) as set forth in (A1)-(A11), and a solubility enhancing agent. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J3) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent, and a preservative. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J4) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent, a preservative, a buffering agent, and a pH modifying agent. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J5) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent, a preservative, and a chelating agent. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J6) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent, a preservative, a buffering agent, a pH modifying agent, and a chelating agent. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J7) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent, a preservative, a buffering agent, a pH modifying agent, a chelating agent, and a viscosity/suspending agent. In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(J8) In another embodiment, formulations for topical administration to the eye of the present application comprise Compound I-A, or a pharmaceutically acceptable salt or solvate thereof (e.g., Compound II-A) as set forth in (A1)-(A11), and a pharmaceutically acceptable excipient as described, where applicable, in any one of (B1)-(B14), (C1)-(C4), (D1)-(D5), (E1)-(E5), (F1)-(F5), (G1)-G3), (H), and (I). In a further embodiment, the formulations comprise Compound I-A, or a pharmaceutically acceptable salt or solvate thereof (e.g., Compound II-A) as set forth in (A3) or (A4). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11).

(K1) In one embodiment, formulations for topical administration to the eye of the present application comprise Compound I-A, or a pharmaceutically acceptable salt or solvate thereof (e.g., Compound II-A), and a solubility enhancing agent as set forth in (B1)-(B14). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B111). In a further embodiment, the formulations comprise a solubility enhancing agent as set forth in (B12)-(B14).

(K2) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), and a preservative. In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(K3) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), a preservative, a pH modifying agent, and a buffering agent. In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(K4) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), a preservative, and a chelating agent. In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(K5) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), a preservative, a pH modifying agent, a buffering agent, and a chelating agent. In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(K6) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), a preservative, a pH modifying agent, a buffering agent, a chelating agent, and a viscosity/suspending agent. In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(K7) In another embodiment, formulations for topical administration to the eye of the present application comprise Compound I-A, or a pharmaceutically acceptable salt or solvate thereof (e.g., Compound II-A), a solubility enhancing agent as set forth in (B1)-(B14), and one or more additional pharmaceutically acceptable excipients as described, where applicable, in any one of (C1)-(C4), (D1)-(D5), (E1)-(E5), (F1)-(F5), (G1)-G3), (H), and (I). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise a solubility enhancing agent as set forth in (B12)-(B14).

(L1) In one embodiment, formulations for topical administration to the eye of the present application comprise Compound I-A, or a pharmaceutically acceptable salt or solvate thereof (e.g., Compound II-A), a solubility enhancing agent, and a preservative as set forth in (D1)-(D5). In a further embodiment, the formulations comprise a preservative as set forth in (D4).

(L2) In another embodiment, formulations for topical administration to the eye of the present application comprise Compound I-A, or a pharmaceutically acceptable salt or solvate thereof (e.g., Compound II-A), a solubility enhancing agent, a preservative as set forth in (D1)-(D5), a pH modifying agent, and a buffering agent. In a further embodiment, the formulations comprise a preservative as set forth in (D4).

(L3) In another embodiment, formulations for topical administration to the eye of the present application comprise Compound I-A, or a pharmaceutically acceptable salt or solvate thereof (e.g., Compound II-A), a solubility enhancing agent, a preservative as set forth in (D1)-(D5), and a chelating agent. In a further embodiment, the formulations comprise a preservative as set forth in (D4).

(L4) In another embodiment, formulations for topical administration to the eye of the present application comprise Compound I-A, or a pharmaceutically acceptable salt or solvate thereof (e.g., Compound II-A), a solubility enhancing agent, a preservative as set forth in (D1)-(D5), a pH modifying agent, a buffering agent, and a chelating agent. In a further embodiment, the formulations comprise a preservative as set forth in (D4).

(L5) In another embodiment, formulations for topical administration to the eye of the present application comprise Compound I-A, or a pharmaceutically acceptable salt or solvate thereof (e.g., Compound II-A), a solubility enhancing agent, a preservative as set forth in (D1)-(D5), a pH modifying agent, a buffering agent, a chelating agent, and a viscosity/suspending agent. In a further embodiment, the formulations comprise a preservative as set forth in (D4).

(L6) In another embodiment, formulations for topical administration to the eye of the present application comprise Compound I-A, or a pharmaceutically acceptable salt or solvate thereof (e.g., Compound II-A), a preservative as set forth in (D1)-(D5), and one or more additional pharmaceutically acceptable excipients as described, where applicable, in any one of (B1)-(B14), (C1)-(C4), (E1)-(E5), (F1)-(F5), (G1)-G3), (H), and (I). In a further embodiment, the formulations comprise a preservative as set forth in (D4).

(M1) In one embodiment, formulations for topical administration to the eye of the present application comprise Compound I-A, or a pharmaceutically acceptable salt or solvate thereof (e.g., Compound II-A), a solubility enhancing agent, and a chelating agent as set forth in (C1)-(C4). In a further embodiment, the formulations comprise a chelating agent as set forth in (C4).

(M2) In another embodiment, formulations for topical administration to the eye of the present application comprise Compound I-A, or a pharmaceutically acceptable salt or solvate thereof (e.g., Compound II-A), a solubility enhancing agent, a preservative, and a chelating agent as set forth in (C1)-(C4). In a further embodiment, the formulations comprise a chelating agent as set forth in (C4).

(M3) In another embodiment, formulations for topical administration to the eye of the present application comprise Compound I-A, or a pharmaceutically acceptable salt or solvate thereof (e.g., Compound II-A), a solubility enhancing agent, a preservative, a pH modifying agent, a buffering agent, and a chelating agent as set forth in (C1)-(C4). In a further embodiment, the formulations comprise a chelating agent as set forth in (C4).

(M4) In another embodiment, formulations for topical administration to the eye of the present application comprise Compound I-A, or a pharmaceutically acceptable salt or solvate thereof (e.g., Compound II-A), a solubility enhancing agent, a preservative, a pH modifying agent, a buffering agent, a chelating agent as set forth in (C1)-(C4), and a viscosity/suspending agent. In a further embodiment, the formulations comprise a chelating agent as set forth in (C4).

(M5) In another embodiment, formulations for topical administration to the eye of the present application comprise Compound I-A, or a pharmaceutically acceptable salt or solvate thereof (e.g., Compound II-A), a chelating agent as set forth in (C1)-(C4), and one or more additional pharmaceutically acceptable excipients as described, where applicable, in any one of (B1)-(B14), (D1)-(D5), (E1)-(E5), (F1)-(F5), (G1)-G3), (H), and (I). In a further embodiment, the formulations comprise a chelating agent as set forth in (C4).

(N1) In one embodiment, formulations for topical administration to the eye of the present application comprise Compound I-A, or a pharmaceutically acceptable salt or solvate thereof (e.g., Compound II-A) as set forth in (A1)-(A11), and a solubility enhancing agent as set forth in (B1)-(B14). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(N2) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and a preservative. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(N3) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, a buffering agent, and a pH modifying agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(N4) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, and a chelating agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(N5) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, a buffering agent, a pH modifying agent, and a chelating agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14).

(N6) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, a buffering agent, a pH modifying agent, a chelating agent, and a viscosity/suspending agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(N7) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and one or more additional pharmaceutically acceptable excipients as described, where applicable, in any one of (C1)-(C4), (D1)-(D5), (E1)-(E5), (F1)-(F5), (G1)-G3), (H), and (I). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In a further embodiment, the formulations comprise Compound I-A, or a pharmaceutically acceptable salt or solvate thereof (e.g., Compound II-A) as set forth in (A5)-(A9). In a further embodiment, the formulations comprise Compound I-A, or a pharmaceutically acceptable salt or solvate thereof (e.g., Compound II-A) as set forth in (A10)-(A11). In a further embodiment, the formulations comprise a solubility enhancing agent as set forth in (B4)-(B7). In a further embodiment, the formulations comprise a solubility enhancing agent as set forth in (B8). In a further embodiment, the formulations comprise a solubility enhancing agent as set forth in (B9). In a further embodiment, the formulations comprise a solubility enhancing agent as set forth in (B10). In a further embodiment, the formulations comprise a solubility enhancing agent as set forth in (B11). In a further embodiment, the formulations comprise Compound I-A, or a pharmaceutically acceptable salt or solvate thereof (e.g., Compound II-A) as set forth in (A5)-(A9) and a solubility enhancing agent as set forth in (B4)-(B7). In a further embodiment, the formulations comprise Compound I-A, or a pharmaceutically acceptable salt or solvate thereof (e.g., Compound II-A) as set forth in (A5) and a solubility enhancing agent as set forth in (B4). In a further embodiment, the formulations comprise Compound I-A, or a pharmaceutically acceptable salt or solvate thereof (e.g., Compound II-A) as set forth in (A6) and a solubility enhancing agent as set forth in (B5). In a further embodiment, the formulations comprise Compound I-A, or a pharmaceutically acceptable salt or solvate thereof (e.g., Compound II-A) as set forth in (A7) and a solubility enhancing agent as set forth in (B6). In a further embodiment, the formulations comprise Compound I-A, or a pharmaceutically acceptable salt or solvate thereof (e.g., Compound II-A) as set forth in (A8) and a solubility enhancing agent as set forth in (B7). In a further embodiment, the formulations comprise Compound I-A, or a pharmaceutically acceptable salt or solvate thereof (e.g., Compound II-A) as set forth in (A8) and a solubility enhancing agent as set forth in (B8).

(O1) In one embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and a preservative as set forth in (D1)-(D5). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(O2) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), a buffering agent, and a pH modifying agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(O3) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), and a chelating agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(O4) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), a buffering agent, a pH modifying agent, and a chelating agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(O5) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), a buffering agent, a pH modifying agent, a chelating agent, and a viscosity/suspending agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(O6) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and a preservative as set forth in (D1)-(D5), and one or more additional pharmaceutically acceptable excipients as described, where applicable, in any one of (C1)-(C4), (E1)-(E5), (F1)-(F5), (G1)-G3), (H), and (I). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4).

(P1) In one embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and a chelating agent as set forth in (C1)-(C4). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4).

(P2) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, and a chelating agent as set forth in (C1)-(C4). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4).

(P3) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, a chelating agent as set forth in (C1)-(C4), a buffering agent, and a pH modifying agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4).

(P4) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative, a chelating agent as set forth in (C1)-(C4), a buffering agent, a pH modifying agent, and a viscosity/suspending agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4).

(P5) In a further embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), and a chelating agent as set forth in (C1)-(C4), and one or more additional pharmaceutically acceptable excipients as described, where applicable, in any one of (D1)-(D5), (E1)-(E5), (F1)-(F5), (G1)-G3), (H), and (I). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in ((B12)-(B14). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4).

(Q1) In one embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), and a chelating agent as set forth in (C1)-(C4). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4). In another further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), a preservative as set forth in (D4), and a chelating agent as set forth in (C4).

(Q2) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), a chelating agent as set forth in (C1)-(C4), a buffering agent, and a pH modifying agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4). In another further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), a preservative as set forth in (D4), and a chelating agent as set forth in (C4).

(Q3) In another embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), a chelating agent as set forth in (C1)-(C4), a buffering agent, a pH modifying agent, and a viscosity/suspending agent. In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4). In another further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), a preservative as set forth in (D4), and a chelating agent as set forth in (C4).

(Q4) In a further embodiment, formulations for topical administration to the eye of the present application comprise Compound I-A, or a pharmaceutically acceptable salt or solvate thereof (e.g., Compound II-A) as set forth in (A1)-(A11), a solubility enhancing agent as set forth in (B1)-(B14), a preservative as set forth in (D1)-(D5), and a chelating agent as set forth in (C1)-(C4), and one or more additional pharmaceutically acceptable excipients as described, where applicable, in any one of (E1)-(E5), (F1)-(F5), (G1)-G3), (H), and (I). In a further embodiment, the formulations comprise Compound I-A, or a pharmaceutically acceptable salt or solvate thereof (e.g., Compound II-A) as set forth in (A3) or (A4). In another further embodiment, the formulations comprise a solubility enhancing agent as set forth in (B12)-(B14). In another further embodiment, the formulations comprise a preservative as set forth in (D4). In another further embodiment, the formulations comprise a chelating agent as set forth in (C4). In a further embodiment, the formulations comprise Compound I-A, or a pharmaceutically acceptable salt or solvate thereof (e.g., Compound II-A) as set forth in (A3) or (A4) and a solubility enhancing agent as set forth in (B12)-(B14). In another further embodiment, the formulations comprise Compound I-A, or a pharmaceutically acceptable salt or solvate thereof (e.g., Compound II-A) as set forth in (A3) or (A4) and a preservative as set forth in (D4). In another further embodiment, the formulations comprise Compound I-A, or a pharmaceutically acceptable salt or solvate thereof (e.g., Compound II-A) as set forth in (A3) or (A4) and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a preservative as set forth in (D4). In another further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), and a chelating agent as set forth in (C4). In a further embodiment, the formulations comprise Compound I-A, or a pharmaceutically acceptable salt or solvate thereof (e.g., Compound II-A) as set forth in (A3) or (A4), a solubility enhancing agent as set forth in (B12)-(B14), a preservative as set forth in (D4), and a chelating agent as set forth in (C4).

(R1) In one embodiment, formulations for topical administration to the eye of the present application comprise Compound I-A, or a pharmaceutically acceptable salt or solvate thereof (e.g., Compound II-A), a solubility enhancing agent, a preservative, and a chelating agent, where applicable, as set forth herein (e.g, in (A1)-(A11), (B1)-(B14), (C1)-(C4), (D1)-(D5), (E1)-(E5), (F1)-(F5), (G1)-G3), (H), and (I)), a buffering agent as set forth in (F1)-(F6), and a pH modifying agent.

(R2) In one embodiment, formulations for topical administration to the eye of the present application comprise Compound I-A, or a pharmaceutically acceptable salt or solvate thereof (e.g., Compound II-A), a solubility enhancing agent, a preservative, and a chelating agent, where applicable, as set forth herein (e.g, in (A1)-(A11), (B1)-(B14), (C1)-(C4), (D1)-(D5), (E1)-(E5), (F1)-(F5), (G1)-G3), (H), and (I)), a buffering agent as set forth in (F1)-(F6), a pH modifying agent, and a viscosity/suspending agent.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 0.5% w/w, greater than about 0.6% w/w, greater than about 0.7% w/w, greater than about 0.8% w/w, greater than about 0.9% w/w, greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 0.5% w/w, greater than about 0.6% w/w, greater than about 0.7% w/w, greater than about 0.8% w/w, greater than about 0.9% w/w, greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 0.5% w/w, greater than about 0.6% w/w, greater than about 0.7% w/w, greater than about 0.8% w/w, greater than about 0.9% w/w, greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the solubility enhancing agent (e.g., cyclodextrin (e.g., HPβCD)) is presented in an amount of greater than about 0.5% w/w, greater than about 1.0% w/w, greater than about 1.5% w/w, greater than about 2.0% w/w, greater than about 2.5% w/w, greater than about 3.0% w/w, greater than about 3.5% w/w, greater than about 4.0% w/w, greater than about 4.1% w/w, greater than about 4.2% w/w, greater than about 4.3% w/w, greater than about 4.4% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the solubility enhancing agent (e.g., cyclodextrin (e.g., HPβCD)) is presented in an amount of greater than about 0.5% w/w, greater than about 1.0% w/w, greater than about 1.5% w/w, greater than about 2.0% w/w, greater than about 2.5% w/w, greater than about 3.0% w/w, greater than about 3.5% w/w, greater than about 4.0% w/w, greater than about 4.1% w/w, greater than about 4.2% w/w, greater than about 4.3% w/w, greater than about 4.4% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) Compound I-A, or the pharmaceutically acceptable salt thereof (e.g., Compound II-A), is presented in an amount greater than about 0.5% w/w, greater than about 0.6% w/w, greater than about 0.7% w/w, greater than about 0.8% w/w, greater than about 0.9% w/w, greater than about 1.0% w/w, greater than about 1.1% w/w, greater than about 1.2% w/w, greater than about 1.3% w/w, greater than about 1.4% w/w, greater than about 1.5% w/w, greater than about 1.6% w/w, greater than about 1.7% w/w, greater than about 1.8% w/w, greater than about 1.9% w/w, greater than about 2% w/w, greater than about 2.1% w/w, greater than about 2.2% w/w, greater than about 2.3% w/w, greater than about 2.4% w/w, greater than about 2.5% w/w, greater than about 2.6% w/w, greater than about 2.7% w/w, greater than about 2.8% w/w, greater than about 2.9% w/w, greater than about 3% w/w, greater than about 3.1% w/w, greater than about 3.2% w/w, greater than about 3.3% w/w, greater than about 3.4% w/w, greater than about 3.5% w/w, greater than about 3.6% w/w, greater than about 3.7% w/w, greater than about 3.8% w/w, greater than about 3.9% w/w, greater than about 4.0% w/w, greater than about 4.5% w/w, greater than about 5.0% w/w, greater than about 5.5% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, or greater than about 10% w/w; and
(ii) the solubility enhancing agent (e.g., cyclodextrin (e.g., HPβCD)) is presented in an amount of greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) the solubility enhancing agent (e.g., cyclodextrin (e.g., HPβCD)) is presented in an amount of greater than about 0.5% w/w, greater than about 1.0% w/w, greater than about 1.5% w/w, greater than about 2.0% w/w, greater than about 2.5% w/w, greater than about 3.0% w/w, greater than about 3.5% w/w, greater than about 4.0% w/w, greater than about 4.1% w/w, greater than about 4.2% w/w, greater than about 4.3% w/w, greater than about 4.4% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) the solubility enhancing agent (e.g., cyclodextrin (e.g., HPβCD)) is presented in an amount of greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 1:1, greater than about 1.2:1, greater than about 1.25:1, greater than about 1.3:1, greater than about 1.4:1, greater than about 1.5:1, greater than about 1.6:1, greater than about 1.7:1, greater than about 1.8:1, greater than about 1.9:1, greater than about 2:1, greater than about 2.1:1, greater than about 2.2:1, greater than about 2.3:1, greater than about 2.4:1, greater than about 2.5:1, greater than about 2.6:1, greater than about 2.7:1, greater than about 2.8:1, greater than about 2.9:1, greater than about 3:1, greater than about 3.1:1, greater than about 3.2:1, greater than about 3.3:1, greater than about 3.4:1, greater than about 3.5:1, greater than about 3.6:1, greater than about 3.7:1, greater than about 3.8:1, greater than about 3.9:1, greater than about 4:1, greater than about 4.5:1, greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, the formulations of the present application comprise Compound I-A, or a pharmaceutically acceptable salt or solvate thereof (e.g., Compound II-A), and a solubility enhancing agent (e.g., cyclodextrin (e.g., HPβCD)), wherein:
(i) the solubility enhancing agent (e.g., cyclodextrin (e.g., HPβCD)) is presented in an amount of greater than about 0.5% w/w, greater than about 1.0% w/w, greater than about 1.5% w/w, greater than about 2.0% w/w, greater than about 2.5% w/w, greater than about 3.0% w/w, greater than about 3.5% w/w, greater than about 4.0% w/w, greater than about 4.1% w/w, greater than about 4.2% w/w, greater than about 4.3% w/w, greater than about 4.4% w/w, greater than about 4.5% w/w, greater than about 4.6% w/w, greater than about 4.7% w/w, greater than about 4.8% w/w, greater than about 4.9% w/w, greater than about 5.0% w/w, greater than about 5.1% w/w, greater than about 5.2% w/w, greater than about 5.3% w/w, greater than about 5.4% w/w, greater than about 5.5% w/w, greater than about 5.6% w/w, greater than about 5.7% w/w, greater than about 5.8% w/w, greater than about 5.9% w/w, greater than about 6.0% w/w, greater than about 6.5% w/w, greater than about 7.0% w/w, greater than about 7.5% w/w, greater than about 8.0% w/w, greater than about 8.5% w/w, greater than about 9.0% w/w, greater than about 9.5% w/w, greater than about 10.0% w/w, greater than about 10.5% w/w, greater than about 11.0% w/w, greater than about 11.5% w/w, greater than about 12.0% w/w, greater than about 12.5% w/w, greater than about 13.0% w/w, greater than about 13.5% w/w, greater than about 14.0% w/w, greater than about 14.5% w/w, greater than about 15.0% w/w, greater than about 15.5% w/w, greater than about 16.0% w/w, greater than about 16.5% w/w, greater than about 17.0% w/w, greater than about 17.5% w/w, greater than about 18.0% w/w, greater than about 18.5% w/w, greater than about 19.0% w/w, greater than about 19.5% w/w, or greater than about 20.0% w/w; and
(ii) the weight ratio between the solubility enhancing agent (e.g., cyclodextrin (e.g., HPβCD)) and Compound I-A, or the pharmaceutically acceptable salt thereof (e.g., Compound II-A), is greater than about 5:1, greater than about 5.5:1, greater than about 6:1, greater than about 6.5:1, greater than about 7:1, greater than about 7.5:1, greater than about 8:1, greater than about 8.5:1, greater than about 9:1, greater than about 9.5:1, or greater than about 10:1.

In one embodiment, formulations for topical administration to the eye of the present application comprise:
Compound I-A, or a pharmaceutically acceptable salt or solvate thereof (e.g., Compound II-A) in an amount between about 7.0% w/w and about 8.0% w/w, between about 7.1% w/w and about 7.9% w/w, between about 7.2% w/w and about 7.8% w/w, between about 7.3% w/w and about 7.7% w/w, or between about 7.4% w/w and about 7.6% w/w (e.g., about 7.5% w/w); and
a cyclodextrin (e.g., HPβCD) in an amount between about 22.7% w/w and about 23.7% w/w, between about 22.8% and about 23.6% w/w, between about 22.9% and about 23.5% w/w, between about 23.0% and about 23.4% w/w, between about 23.1% and about 23.3% w/w, or between about 23.2% and about 23.3% w/w (e.g., about 23.25% w/w).

In one embodiment, formulations for topical administration to the eye of the present application comprise:
Compound I-A, or a pharmaceutically acceptable salt or solvate thereof (e.g., Compound II-A) in an amount between about 4.2% w/w and about 5.2% w/w, between about 4.3% w/w and about 5.1% w/w, between about 4.4% w/w and about 5.0% w/w, between about 4.5% w/w and about 4.9% w/w, or between about 4.6% w/w and about 4.8% w/w (e.g., about 4.7% w/w); and
a cyclodextrin (e.g., HPβCD) in an amount between about 14.1% w/w and about 15.1% w/w, between about 14.2% w/w and about 15.0% w/w, between about 14.3% w/w and about 14.9% w/w, between about 14.4% w/w and about 14.8% w/w, or between about 14.5% w/w and about 14.7% w/w (e.g., about 14.6% w/w).

In one embodiment, formulations for topical administration to the eye of the present application comprise:
Compound I-A, or a pharmaceutically acceptable salt or solvate thereof (e.g., Compound II-A) in an amount between about 1.9% w/w and about 2.9% w/w, between about 2.0% w/w and about 2.8% w/w, between about 2.1% w/w and about 2.7% w/w, between about 2.2% w/w and about 2.6% w/w, or between about 2.3% w/w and about 2.5% w/w (e.g., about 2.4% w/w); and
a cyclodextrin (e.g., HPβCD) in an amount between about 7.0% w/w and about 8.0% w/w, between about 7.1% w/w and about 7.9% w/w, between about 7.2% w/w and about 7.8% w/w, between about 7.3% w/w and about 7.7% w/w, or between about 7.4% w/w and about 7.6% w/w (e.g., about 7.5% w/w).

In one embodiment, formulations for topical administration to the eye of the present application comprise:
Compound I-A, or a pharmaceutically acceptable salt or solvate thereof (e.g., Compound II-A) in an amount between about 0.5% w/w and about 1.5% w/w, between about 0.6% w/w and about 1.4% w/w, between about 0.7% w/w and about 1.3% w/w, between about 0.8% w/w and about 1.2% w/w, or between about 0.9% w/w and about 1.1% w/w (e.g., about 1.0% w/w); and
a cyclodextrin (e.g., HPβCD) in an amount between about 2.6% w/w and about 3.6% w/w, between about 2.7% w/w and about 3.5% w/w, between about 2.8% w/w and about 3.4% w/w, between about 2.9% w/w and about 3.3% w/w, or between about 3.0% w/w and about 3.2% w/w (e.g., about 3.1% w/w).

In one embodiment, formulations for topical administration to the eye of the present application further comprises a preservative.

In one embodiment, formulations for topical administration to the eye of the present application further comprises a chelating agent.

In one embodiment, formulations for topical administration to the eye of the present application further comprises a buffering agent.

In one embodiment, formulations for topical administration to the eye of the present application comprise:
Compound I-A, or a pharmaceutically acceptable salt or solvate thereof (e.g., Compound II-A) in an amount of about 0.5% w/w, about 1.25% w/w, about 2.5% w/w, about 5.0% w/w, about 7.5% w/w, about 10% w/w, about 12.5% w/w, about 15% w/w, about 17.5% w/w, or about 20% w/w;
a solubility enhancing agent in an amount of about 2.5% w/w or about 5.0% w/w;
a preservative in an amount of about 0.005% w/w, about 0.01% w/w, about 0.015% w/w, or about 0.02% w/w;
a chelating agent in an amount of about 0.1% w/w; and
a buffering agent in an amount of about 1% w/w.

In a further embodiment, the formulations may further comprise a pH modifying agent. In a further embodiment, the formulations may further comprise a viscosity/suspending agent.

In one embodiment, formulations for topical administration to the eye of the present application comprise:
Compound I-A, or a pharmaceutically acceptable salt or solvate thereof (e.g., Compound II-A) in an amount of about 0.5% w/w, about 1.25% w/w, about 2.5% w/w, or about 5.0% w/w;
a solubility enhancing agent in an amount of about 2.5% w/w or about 5.0% w/w;
a preservative in an amount of about 0.005% w/w, about 0.01% w/w, about 0.015% w/w, or about 0.02% w/w;
a chelating agent in an amount of about 0.1% w/w; and
a buffering agent in an amount of about 1% w/w.

In a further embodiment, the formulations may further comprise a pH modifying agent. In a further embodiment, the formulations may further comprise a viscosity/suspending agent.

In one embodiment, formulations for topical administration to the eye of the present application comprise:
Compound I-A, or a pharmaceutically acceptable salt or solvate thereof (e.g., Compound II-A) in an amount of about 0.5% w/w, about 1.25% w/w, about 2.5% w/w, about 5.0% w/w, about 7.5% w/w, about 10% w/w, about 12.5% w/w, about 15% w/w, about 17.5% w/w, or about 20% w/w;
a cyclodextrin in an amount of about 2.5% w/w or about 5.0% w/w;
a BAC in an amount of about 0.005% w/w, about 0.01% w/w, about 0.015% w/w, or about 0.02% w/w;
an EDTA in an amount of about 0.1% w/w; and
a boric acid or citrate in an amount of about 1% w/w.

In a further embodiment, the formulations may further comprise a pH modifying agent. In a further embodiment, the formulations may further comprise a viscosity/suspending agent. In a further embodiment, the viscosity/suspending agent is PEG 6000 or PVP.

In one embodiment, formulations for topical administration to the eye of the present application comprise:
Compound I-A, or a pharmaceutically acceptable salt or solvate thereof (e.g., Compound II-A) in an amount of about 0.5% w/w, about 1.25% w/w, about 2.5% w/w, or about 5.0% w/w;
a cyclodextrin in an amount of about 2.5% w/w or about 5.0% w/w;
a BAC in an amount of about 0.005% w/w, about 0.01% w/w, about 0.015% w/w, or about 0.02% w/w;
an EDTA in an amount of about 0.1% w/w; and
a boric acid or citrate in an amount of about 1% w/w.

In a further embodiment, the formulations may further comprise a pH modifying agent. In a further embodiment, the formulations may further comprise a viscosity/suspending agent. In a further embodiment, the viscosity/suspending agent is PEG 6000 or PVP.

In one embodiment, formulations for topical administration to the eye of the present application comprise:
Compound I-A, or a pharmaceutically acceptable salt or solvate thereof (e.g., Compound II-A) in an amount of about 0.5% w/w, about 1.25% w/w, about 2.5% w/w, about 5.0% w/w, about 7.5% w/w, about 10% w/w, about 12.5% w/w, about 15% w/w, about 17.5% w/w, or about 20% w/w;
a cyclodextrin in an amount of about 5.0% w/w;
a BAC in an amount of about 0.02% w/w;
a EDTA in an amount of about 0.1% w/w; and
a boric acid or citrate in an amount of about 1% w/w.

In a further embodiment, the formulations may further comprise a pH modifying agent. In a further embodiment, the formulations may further comprise a viscosity/suspending agent. In a further embodiment, the viscosity/suspending agent is PEG 6000 or PVP.

In one embodiment, formulations for topical administration to the eye of the present application comprise:
Compound I-A, or a pharmaceutically acceptable salt or solvate thereof (e.g., Compound II-A) in an amount of about 2.5% w/w or about 5.0% w/w;
a cyclodextrin in an amount of about 5.0% w/w;
a BAC in an amount of about 0.02% w/w;
a EDTA in an amount of about 0.1% w/w; and
a boric acid or citrate in an amount of about 1% w/w.

In a further embodiment, the formulations may further comprise a pH modifying agent. In a further embodiment, the formulations may further comprise a viscosity/suspending agent. In a further embodiment, the viscosity/suspending agent is PEG 6000 or PVP.

In one embodiment, formulations for topical administration to the eye of the present application comprise:
Compound I-A, or a pharmaceutically acceptable salt or solvate thereof (e.g., Compound II-A) in an amount of about 7.5% w/w; and
a cyclodextrin (e.g., HPβCD) in an amount of about 23.25% w/w.

In one embodiment, formulations for topical administration to the eye of the present application comprise:
Compound I-A, or a pharmaceutically acceptable salt or solvate thereof (e.g., Compound II-A) in an amount of about 7.5% w/w;
a cyclodextrin (e.g., HPβCD) in an amount of about 23.25% w/w;
a BAC;
a EDTA (e.g., Na-EDTA); and
a boric acid.

In one embodiment, formulations for topical administration to the eye of the present application comprise:
Compound I-A, or a pharmaceutically acceptable salt or solvate thereof (e.g., Compound II-A) in an amount of about 7.5% w/w;
a cyclodextrin (e.g., HPβCD) in an amount of about 23.25% w/w;
a BAC in an amount of about 0.02% w/w;
a EDTA (e.g., Na-EDTA) in an amount of about 0.1% w/w; and
a boric acid in an amount of about 1% w/w.

In one embodiment, formulations for topical administration to the eye of the present application comprise:
Compound I-A, or a pharmaceutically acceptable salt or solvate thereof (e.g., Compound II-A) in an amount of about 4.7% w/w; and
a cyclodextrin (e.g., HPβCD) in an amount of about 14.6% w/w.

In one embodiment, formulations for topical administration to the eye of the present application comprise:
Compound I-A, or a pharmaceutically acceptable salt or solvate thereof (e.g., Compound II-A) in an amount of about 4.7% w/w;
a cyclodextrin (e.g., HPβCD) in an amount of about 14.6% w/w;
a BAC;
a EDTA (e.g., Na-EDTA); and
a boric acid.

In one embodiment, formulations for topical administration to the eye of the present application comprise:
Compound I-A, or a pharmaceutically acceptable salt or solvate thereof (e.g., Compound II-A) in an amount of about 4.7% w/w;
a cyclodextrin (e.g., HPβCD) in an amount of about 14.6% w/w;
a BAC in an amount of about 0.02% w/w;
a EDTA (e.g., Na-EDTA) in an amount of about 0.1% w/w; and
a boric acid in an amount of about 1% w/w.

In one embodiment, formulations for topical administration to the eye of the present application comprise:
Compound I-A, or a pharmaceutically acceptable salt or solvate thereof (e.g., Compound II-A) in an amount of about 2.4% w/w; and
a cyclodextrin (e.g., HPβCD) in an amount of about 7.5% w/w.

In one embodiment, formulations for topical administration to the eye of the present application comprise:
Compound I-A, or a pharmaceutically acceptable salt or solvate thereof (e.g., Compound II-A) in an amount of about 2.4% w/w;
a cyclodextrin (e.g., HPβCD) in an amount of about 7.5% w/w;
a BAC;
a EDTA (e.g., Na-EDTA); and
a boric acid.

In one embodiment, formulations for topical administration to the eye of the present application comprise:
Compound I-A, or a pharmaceutically acceptable salt or solvate thereof (e.g., Compound II-A) in an amount of about 2.4% w/w;
a cyclodextrin (e.g., HPβCD) in an amount of about 7.5% w/w;
a BAC in an amount of about 0.02% w/w;
a EDTA (e.g., Na-EDTA) in an amount of about 0.1% w/w; and
a boric acid in an amount of about 1% w/w.

In one embodiment, formulations for topical administration to the eye of the present application comprise:
Compound I-A, or a pharmaceutically acceptable salt or solvate thereof (e.g., Compound II-A) in an amount of about 1.0% w/w; and
a cyclodextrin (e.g., HPβCD) in an amount of about 3.1% w/w.

In one embodiment, formulations for topical administration to the eye of the present application comprise:
Compound I-A, or a pharmaceutically acceptable salt or solvate thereof (e.g., Compound II-A) in an amount of about 1.0% w/w;
a cyclodextrin (e.g., HPβCD) in an amount of about 3.1% w/w;
a BAC;
a EDTA (e.g., Na-EDTA); and
a boric acid.

In one embodiment, formulations for topical administration to the eye of the present application comprise:
Compound I-A, or a pharmaceutically acceptable salt or solvate thereof (e.g., Compound II-A) in an amount of about 1.0% w/w;
a cyclodextrin (e.g., HPβCD) in an amount of about 3.1% w/w;
a BAC in an amount of about 0.02% w/w;
a EDTA (e.g., Na-EDTA) in an amount of about 0.1% w/w; and
a boric acid in an amount of about 1% w/w.

### O. Exemplary Further Features of the Formulations

The formulations may be further characterized to additional features described herein below. For example, in certain embodiments, the formulation inhibits or decreases formation of blood vessels in the eye or in a tissue from the eye, in vivo or in vitro. In one embodiment, a formulation of the present application decreases the formation of blood vessels below 90%, 80%, 70%, 60%, 50%, 40%, 30%, 20%, 10%, or 5%, as compared to that in an untreated control. In a further embodiment, a formulation of the present application decreases the formation of blood vessels below 60%, 50%, 40%, 30%, 20%, 10%, or 5%, as compared to that in an untreated control. In a further embodiment, a formulation of the present application decreases the formation of blood vessels below 40%, 30%, 20%, 10%, or 5%, as compared to that in an untreated control.

In one embodiment, a formulation of the present application is efficiently distributed to the back of the eye, e.g., retina, after topical administration. In one embodiment, a formulation of the present application is efficiently distributed to the retina within 12 hours, within 10 hours, within 8 hours, within 6 hours, within 4 hours, within 2 hours, or within 1 hour, after topical administration to the eye. In a further embodiment, a formulation of the present application is efficiently distributed to the retina within 8 hours, within 6 hours, within 4 hours, within 2 hours, or within 1 hour, after topical administration to the eye. In a further embodiment, a formulation of the present application is efficiently distributed to the retina within 2 hours or within 1 hour, after topical administration to the eye.

In one embodiment, a formulation of the present application is efficiently distributed into the eye, e.g., choroid, after topical administration. In one embodiment, a formulation of the present application is efficiently distributed to the choroid within 12 hours, within 10 hours, within 8 hours, within 6 hours, within 4 hours, within 2 hours, or within 1 hour, after topical administration to the eye. In a further embodiment, a formulation of the present application is efficiently distributed to the choroid within 8 hours, within 6 hours, within 4 hours, within 2 hours, or within 1 hour, after topical administration to the eye. In a further embodiment, a formulation of the present application is efficiently distributed to the choroid within 2 hours or within 1 hour, after topical administration to the eye.

In one embodiment, a formulation of the present application is efficiently distributed into the compartments of the eye, e.g., aqueous humor and/or sclera, after topical administration. In one embodiment, a formulation of the present application is efficiently distributed to the compartments of the eye within 12 hours, within 10 hours, within 8 hours, within 6 hours, within 4 hours, within 2 hours, or within 1 hour, after topical administration to the eye. In a further embodiment, a formulation of the present application is efficiently distributed to the compartments of the eye within 8 hours, within 6 hours, within 4 hours, within 2 hours, or within 1 hour, after topical administration to the eye. In a further embodiment, a formulation of the present application is efficiently distributed to the compartments of the eye within 2 hours or within 1 hour, after topical administration to the eye.

Also provided are a unit dose of the ophthalmic formulation described herein. In certain embodiments, the unit dose has a volume less than 10 mL. In certain embodiments, the unit dose has a volume less than 5 mL. In certain embodiments, the unit dose has a volume less than 2 mL. In certain embodiments, the unit dose has a volume of about 1, 2, 3, 4, 5, 6, 7, 8, 9, or 10 mL. In certain embodiments, the unit dose is sealed in a container that contains an inert gas, e.g., nitrogen or argon.

### III. PREPARATION OF OPHTHALMIC FORMULATIONS

Another aspect of the invention provides methods for preparing an ophthalmic formulations. For example, in one aspect, the invention provides a method of preparing an aqueous, ophthalmic solution, wherein the method comprises:
a. providing a first mixture comprising water, a cyclodextrin, and a compound of Formula I:

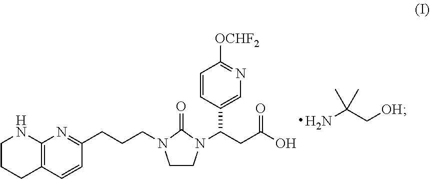

b. admixing a preservative and the first mixture, to thereby provide the aqueous, ophthalmic solution.

In certain embodiments, the aqueous, ophthalmic solution has a pH in the range of 7.5 to 8.7.

In certain embodiments, the preservative is benzalkonium halide. In certain embodiments, the preservative is benzalkonium chloride.

In certain embodiments, the first mixture further comprises a buffer. In certain embodiments, the buffer comprises an organic acid. In certain embodiments, the buffer comprises boric acid.

In certain embodiments, the solution comprises at least 75% w/w water.

In certain embodiments, the method is further characterized according to a feature described in Section II above.

### IV. HYDRATE OF (3)-3-(6-(DIFLUOROMETHOXY)PYRIDIN-3-YL)-3-(2-OXO-3-(3-(5,6,7,8-TETRAHYDRO-1,8-NAPHTHYRIDIN-2-YL)PROPYL)IMIDAZOLIDIN-1-YL)PROPANOIC ACID HYDRATE

Another aspect of the invention provides a hydrate of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid. In certain embodiments, the compound is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

In certain embodiments, the hydrate compound has the following formula:

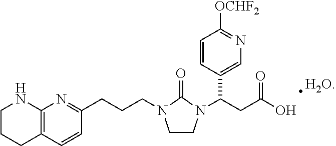 [SMILES: O=C(O)C[C@@H](c1ccc(OC(F)F)nc1)N1CCN(CCCc2ccc3c(n2)NCCC3)C1=O ; "H2O."]

In certain embodiments, the compound is in crystalline form.

Another aspect of the invention provides a pharmaceutical composition comprising a pharmaceutically acceptable carrier and a hydrate compound having the following formula:

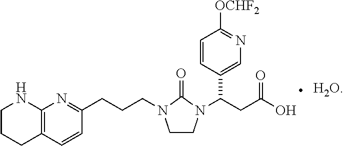 [SMILES: O=C(O)C[C@@H](c1ccc(OC(F)F)nc1)N1CCN(CCCc2ccc3c(n2)NCCC3)C1=O ; "H2O."]

### V. THERAPEUTIC APPLICATIONS

Another aspect of the invention provides a method of treating a disorder mediated by an αv integrin, wherein the method comprises topically administering to an eye of a subject in need thereof a therapeutically effective amount of a solution described herein to treat the disorder.

In certain embodiments, the αv integrin is an αvβ3 or αvβ5 integrin. In certain embodiments, the disorder is macular degeneration, diabetic retinopathy, macular edema, diabetic macular edema, or macular edema following retinal vein occlusion. In certain embodiments, the disorder is diabetic retinopathy. In certain embodiments, the subject is an adult human.

Another aspect of the invention provides a method of inhibiting the activity of an αv integrin in a subject, wherein the method comprises topically administering to an eye of a subject in need thereof an effective amount of a solution described herein to inhibit the activity of the αv integrin. In certain embodiments, the αv integrin is an αvβ3 or αvβ5 integrin. In certain embodiments, the subject is suffering from macular degeneration, diabetic retinopathy, macular edema, diabetic macular edema, or macular edema following retinal vein occlusion. In certain embodiments, the subject is suffering from diabetic retinopathy. In certain embodiments, the subject is an adult human.

Another aspect of the invention provides a method of inhibiting the formation of blood vessels in the eye of a subject, wherein the method comprises topically administering to an eye of a subject in need thereof an effective amount of a solution described herein to inhibit the formation of blood vessels in the eye of the subject. In certain embodiments, the subject is suffering from macular degeneration, diabetic retinopathy, macular edema, diabetic macular edema, or macular edema following retinal vein occlusion. In certain embodiments, the subject is suffering from diabetic retinopathy. In certain embodiments, the subject is an adult human.

As generally described above, the formulations described herein are useful for the treatment or prevention of a disorder mediated by an αv integrin, such as αvβ3 and/or αvβ5 integrin. In a further embodiment, the disorder is a disorder in which angiogenesis is involved. In a further embodiment, the disorder is a disorder in which ocular angiogenesis is involved. In one embodiment, the disorder is macular degeneration, AMD, DR, DME, or macular edema following RVO.

The present application provides a method of treating or preventing a disorder, comprising administering to a subject in need thereof a therapeutically effective amount of a formulation of the present application for topical administration to the eye of the subject. In one embodiment, the present application provides treating a disorder. In one embodiment, the present application provides preventing a disorder.

The present application further provides a method of treating or preventing AMD, DR, DME, or macular edema following RVO, comprising administering to a subject in need thereof, a therapeutically effective amount of a formulation of the present application for topical administration to the eye of the subject. In one embodiment, the present application provides treating AMD, DR, DME, or macular edema following RVO. In one embodiment, the present application provides preventing AMD, DR, DME, or macular edema following RVO.

The present application further provides a method of treating or preventing a disorder, comprising administering to a subject in need thereof a therapeutically effective amount of a formulation of the present application for topical administration to the eye of the subject, in combination with one or more therapies for treating or preventing the disorder. In one embodiment, the therapy is an anti-VEGF therapy. In a further embodiment, the anti-VEGF therapy is intravitreally injected.

The present application provides a formulation of the present application for use in treating or preventing a disorder in a subject. In one embodiment, the formulation of the present application is for use in topical administration to the eye. In one embodiment, the formulation of the present application is for use in combination with one or more therapies for treating or preventing the disorder.

The present application provides a formulation of the present application for use in the manufacture of a medicament for the treatment or prevention of a disorder in a subject. In one embodiment, the medicament is formulated for topical administration to the eye. In one embodiment, the medicament is for use in combination with one or more therapies for treating or preventing the disorder.

The present application provides use of a formulation of the present application in the manufacture of a medicament for the treatment or prevention of a disorder in a subject. In one embodiment, the medicament is formulated for topical administration to the eye. In one embodiment, the medicament is for use in combination with one or more therapies for treating or preventing the disorder.

In one embodiment, the present application provides treatment of a disorder. In one embodiment, the present application provides prevention of a disorder.

In one embodiment, the disorder is an ocular disorder. In one embodiment, the disorder is mediated by an αv integrin. In one embodiment, the disorder is mediated by an αvβ3 and/or αvβ5 integrin. In one embodiment, the disorder is a disorder in which angiogenesis is involved. In one embodiment, the disorder is a disorder in which ocular angiogenesis is involved. In one embodiment, the disorder is macular degeneration. In one embodiment, the disorder is age-related macular degeneration (AMD). In one embodiment, the disorder is diabetic retinopathy (DR). In one embodiment, the disorder is diabetic macular edema (DME). In one embodiment, the disorder is macular edema following retinal vein occlusion (RVO).

Inhibiting the function of αvβ3 and αvβ5 integrins prevents endothelial cell proliferation. Endothelial cell proliferation can result in deleterious neovascularization or angiogenesis, particularly choroidal neovascularization in the choriocapillaris, through Bruch's membrane, ultimately leading to blood and protein leakage below the macula. Bleeding, leaking, and scarring from these blood vessels eventually cause irreversible damage to the photoreceptors and rapid vision loss if left untreated.

Diabetic retinopathy, a closely related condition, is the result of microvascular retinal changes. Hyperglycemia-induced intramural pericyte death and thickening of the basement membrane lead to incompetence of the vascular walls in the retina, which affects the blood-retinal barrier and makes the retinal blood vessels more permeable. Damaged blood vessels leak fluid and lipids onto the macula, the part of the retina that provides us with detailed vision, causing the macula to swell. Eventually this can progress to develop a condition called macular edema.

Accordingly, AMD, DR, DME, and macular edema following central retinal vein occlusion (thrombosis) can be treated or prevented through administration (e.g., topical administration) of the formulations of the present application.

In one embodiment, the formulations of the present application are administered topically to the eye. In a further embodiment, the formulation of the present application is administered as an ophthalmic solution. In another embodiment, the formulation of the present application is administered as an ophthalmic emulsion, suspension, gel, or semi-gel. In another embodiment, the formulation of the present application is administered as an ophthalmic jelly, oil, ointment, cream, or spray.

The formulations of the present application are administered in dosages effective to inhibit the function of αvβ3 and/or αvβ5 integrins and thus treat or prevent a disease condition mediated by the αvβ3 and/or αvβ5 integrin.

The present application provides a method of treating or preventing a disorder mediated by an αv integrin in a subject, comprising administering to a subject in need thereof a therapeutically effective amount of a formulation of the present application. In one embodiment, the disorder is a disorder in which angiogenesis is involved. In a further embodiment, the disorder is a disorder in which ocular angiogenesis is involved.

The present application also provides a method of treating or preventing an αvβ3 and/or αvβ5 integrin-mediated disorder in a subject, comprising administering to a subject in need thereof a therapeutically effective amount of a formulation of the present application. In one embodiment, the disorder is a disorder in which ocular angiogenesis is involved. In one embodiment, the disorder is macular degeneration. In one embodiment, the disorder is age-related macular degeneration (AMD). In one embodiment, the disorder is diabetic retinopathy (DR). In one embodiment, the disorder is diabetic macular edema (DME). In one embodiment, the disorder is macular edema following retinal vein occlusion (RVO).

The present application further provides a method of treating or preventing AMD, DR, DME, or macular edema following RVO, comprising administering to a subject in need thereof, a therapeutically effective amount of a formulation of the present application. In one embodiment, the present application provides treating AMD, DR, DME, or macular edema following RVO. In one embodiment, the present application provides preventing AMD, DR, DME, or macular edema following RVO.

The present application further provides a method of treating or preventing a disorder in a subject that is mediated by an αv integrin, comprising administering to a subject in need thereof a therapeutically effective amount of a formulation of the present application, in combination with a second therapy for treating or preventing the disorder. In one embodiment, the disorder is mediated by an αv integrin. In a further embodiment, the disorder is mediated by an αvβ3 and/or αvβ5 integrin. In one embodiment, the disorder is a disorder in which angiogenesis is involved. In a further embodiment, the disorder is a disorder in which ocular angiogenesis is involved.

The second therapy can be administered via any administration routes, including oral administration in forms such as tablets, capsules (each of which includes sustained release or timed release formulations), pills, powders, granules, elixirs, tinctures, suspensions, syrups emulsions, intravenous administration (bolus or in-fusion), intraperitoneal administration, topical administration (e.g., ocular eye-drop), subcutaneous administration, intramuscular administration, transdermal (e.g., patch) administration, and intravitreal administration. In one embodiment, the second therapy is administered through intravitreal injection.

Administration of the second therapy in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present application. “Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner.

The dosage regimen utilizing the formulations of the present application is selected in accordance with a variety of factors including type, species, age, weight, sex and medical condition of the patient; the severity of the condition to be treated; and the particular compound or salt thereof employed.

Another aspect of the invention provides a method of treating a disorder mediated by an αv integrin, wherein the method comprises topically administering to an eye of a subject in need thereof a therapeutically effective amount of a hydrate of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid described herein to treat the disorder.

In certain embodiments, the αv integrin is an αvβ3 or αvβ5 integrin. In certain embodiments, the disorder is macular degeneration, diabetic retinopathy, macular edema, diabetic macular edema, or macular edema following retinal vein occlusion. In certain embodiments, the disorder is diabetic retinopathy. In certain embodiments, the subject is an adult human.

Another aspect of the invention provides a method of inhibiting the activity of an αv integrin in a subject, wherein the method comprises topically administering to an eye of a subject in need thereof an effective amount of a hydrate of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid described herein to inhibit the activity of the αv integrin. In certain embodiments, the αv integrin is an αvβ3 or αvβ5 integrin. In certain embodiments, the subject is suffering from macular degeneration, diabetic retinopathy, macular edema, diabetic macular edema, or macular edema following retinal vein occlusion. In certain embodiments, the subject is suffering from diabetic retinopathy. In certain embodiments, the subject is an adult human.

Another aspect of the invention provides a method of inhibiting the formation of blood vessels in the eye of a subject, wherein the method comprises topically administering to an eye of a subject in need thereof an effective amount of a hydrate of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid described herein to inhibit the formation of blood vessels in the eye of the subject. In certain embodiments, the subject is suffering from macular degeneration, diabetic retinopathy, macular edema, diabetic macular edema, or macular edema following retinal vein occlusion. In certain embodiments, the subject is suffering from diabetic retinopathy. In certain embodiments, the subject is an adult human.

### Medical Uses

Another aspect of the invention provides for the use of a formulation described herein (such as a formulation in Section I) in the manufacture of a medicament. In certain embodiments, the medicament is for treating a disorder described herein, such as diabetic retinopathy.

Another aspect of the invention provides for the use of a formulation described herein (such as a formulation in Section I) for treating a medical disorder, such as a medical disorder described herein, such as diabetic retinopathy.

### VI. KITS FOR USE IN MEDICAL APPLICATIONS

Another aspect of the invention provides a kit for treating a disorder. The kit comprises: i) instructions for treating a disorder described herein, such as diabetic retinopathy; and ii) a formulation described herein.

The description above describes multiple aspects and embodiments of the invention. The patent application specifically contemplates all combinations and permutations of the aspects and embodiments.

### VII. EXEMPLARY ENUMERATED EMBODIMENTS

Exemplary enumerated embodiments are set forth below:

Embodiment no. 1. A formulation for topical administration to the eye of a subject in need thereof, comprising an αv integrin antagonist having the structure:

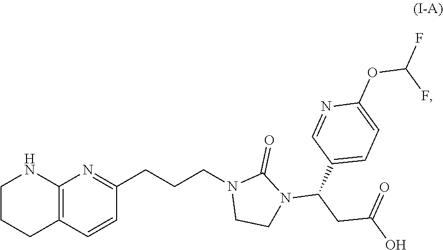 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof.

Embodiment no. 2. The formulation of embodiment 1, wherein the αv integrin antagonist has the structure:

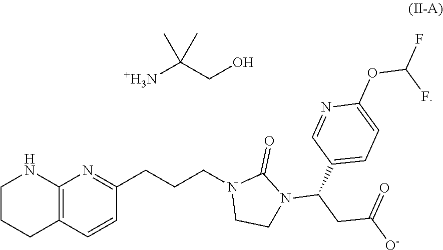

Embodiment no. 3. The formulation of embodiment 1 or 2, wherein the αv integrin antagonist is present in an amount between about 0.05% w/w and about 30.0% w/w.

Embodiment no. 4. The formulation of embodiment 3, wherein the αv integrin antagonist is present in an amount of about 0.5% w/w, about 1.25% w/w, about 2.5% w/w, about 5.0% w/w, or about 7.5% w/w.

Embodiment no. 5. The formulation of embodiment 3, wherein the αv integrin antagonist is present in an amount between about 7.0% w/w and about 8/0% w/w, between about 4.2% w/w and about 5.2% w/w, between about 1.9% w/w and about 2.9% w/w, or between about 0.5% w/w and about 1.5% w/w.

Embodiment no. 6. The formulation embodiment 3, wherein the αv integrin antagonist is present in an amount of greater than about 0.5% w/w.

Embodiment no. 7. The formulation of any one of the preceding embodiments, further comprising a solubility enhancing agent.

Embodiment no. 8. The formulation of embodiment 7, wherein the solubility enhancing agent is a cyclodextrin.

Embodiment no. 9. The formulation of embodiment 8, wherein the cyclodextrin is selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and a mixture thereof.

Embodiment no. 10. The formulation of embodiment 9, wherein the cyclodextrin is a β-cyclodextrin sulfobutyl ether or hydroxypropyl-β-cyclodextrin.

Embodiment no. 11. The formulation of embodiment 10, wherein the cyclodextrin is hydroxypropyl-β-cyclodextrin.

Embodiment no. 12. The formulation any one of embodiments 7-11, wherein the solubility enhancing agent is present in an amount between about 0.5% w/w and about 25.0% w/w.

Embodiment no. 13. The formulation of embodiment 12, wherein the solubility enhancing agent is present in an amount from about 2.5% w/w to about 3.5% w/w, about 7.0% w/w to about 8.0% w/w, about 14.0% w/w to about 15.0% w/w, or about 23.0% w/w to about 24.0% w/w.

Embodiment no. 14. The formulation of embodiment 12, wherein the solubility enhancing agent is present in an amount of greater than about 0.5% w/w.

Embodiment no. 15. The formulation of embodiment 12, wherein weight ratio between the solubility enhancing agent and the αv integrin antagonist is greater than about 1:1.

Embodiment no. 16. The formulation of embodiment 12, wherein weight ratio between the solubility enhancing agent and the αv integrin antagonist is from about 1:1 to about 6:1.

Embodiment no. 17. The formulation of embodiment 12, wherein molar ratio between the solubility enhancing agent and the αv integrin antagonist is greater than about 0.8:1.

Embodiment no. 18. The formulation of embodiment 12, wherein molar ratio between the solubility enhancing agent and the αv integrin antagonist is from about 0.8:1 to about 5:1.

Embodiment no. 19. The formulation of any one of the preceding embodiments, further comprising an excipient selected from the group consisting of a preservative, and a buffering agent, and a mixture thereof.

Embodiment no. 20. The formulation of embodiment 19, wherein the preservative is selected from the group consisting of benzalkonium halide, benzoxonium halide, chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, polyaminopropyl biguanide, and butyl-p-hydroxybenzoate, and sorbic acid, and a mixture thereof.

Embodiment no. 21. The formulation of embodiment 20, wherein the preservative is a benzalkonium halide or benzoxonium halide.

Embodiment no. 22. The formulation of embodiment 21, wherein the benzalkonium halide or benzoxonium halide is benzalkonium chloride or benzoxonium chloride.

Embodiment no. 23. The formulation of any one of embodiments 19-22, wherein the preservative is present in an amount between about 0.001% w/w and about 0.5% w/w.

Embodiment no. 24. The formulation of embodiment 23, wherein the preservative is present in an amount of about 0.005% w/w, about 0.01% w/w, about 0.015% w/w, or about 0.02% w/w.

Embodiment no. 25. The formulation of embodiment 19, wherein the buffering agent is boric acid or a citrate buffer.

Embodiment no. 26. The formulation of embodiment 25, wherein the buffering agent is present in an amount between about 0.01% w/w and about 10% w/w.

Embodiment no. 27. The formulation of embodiment 26, wherein the buffering agent is present in an amount of about 1% w/w.

Embodiment no. 28. The formulation of any one of the preceding embodiments, further comprising an excipient selected from the group consisting of a chelating agent, a tonicity agent, a viscosity/suspending agent, and a pH modifying agent, and a mixture thereof.

Embodiment no. 29. The formulation of embodiment 28, wherein the chelating agent is EDTA.

Embodiment no. 30. The formulation of embodiment 29, wherein chelating agent is present in an amount of about 0.001% w/w to about 1.0% w/w.

Embodiment no. 31. The formulation of embodiment 30, wherein chelating agent is present in an amount of about 0.1% w/w.

Embodiment no. 32. The formulation of embodiment 28, wherein the viscosity/suspending agent is selected from the group consisting of methyl cellulose, ethyl cellulose, hydroxyethylcellulose, polyethylene glycol, polyvinylpyrrolidone, carboxymethyl cellulose, hydroxypropylmethyl cellulose, and cross-linked acrylic acid polymers, and a mixture thereof.

Embodiment no. 33. The formulation of embodiment 32, wherein the viscosity/suspending agent is polyethylene glycol or polyvinylpyrrolidone.

Embodiment no. 34. The formulation of any one of embodiments 7-33, wherein:
(i) the αv integrin antagonists presented in an amount greater than about 0.5% w/w, greater than about 0.6% w/w; and
(ii) the weight ratio between the solubility enhancing agent and the αv integrin antagonists greater than about 1:1.

Embodiment no. 35. The formulation of any one of embodiments 7-33, wherein:
(i) the αv integrin antagonist is presented in an amount greater than about 2.5% w/w; and
(ii) the weight ratio between the solubility enhancing agent and the αv integrin antagonist is greater than about 1:1.

Embodiment no. 36. The formulation of any one of embodiments 7-33, wherein:
(i) the αv integrin antagonist is presented in an amount greater than about 1.0% w/w; and
(ii) the weight ratio between the solubility enhancing agent and the αv integrin antagonist is greater than about greater than about 2:1.

Embodiment no. 37. The formulation of any one of embodiments 7-33, wherein:
(i) the αv integrin antagonist is presented in an amount greater than about 0.5% w/w; and
(ii) the weight ratio between the solubility enhancing agent and the αv integrin antagonist is greater than about 5:1.

Embodiment no. 38. The formulation of any one of embodiments 7-33, wherein:
(i) the αv integrin antagonist is presented in an amount greater than about 0.5% w/w; and
(ii) the solubility enhancing agent is presented in an amount of greater than about 0.5% w/w.

Embodiment no. 39. The formulation of any one of embodiments 7-33, wherein:
(i) the αv integrin antagonist is presented in an amount greater than about 5.0% w/w; and
(ii) the solubility enhancing agent is presented in an amount of greater than about 0.5% w/w.

Embodiment no. 40. The formulation of any one of embodiments 7-33, wherein:
(i) the αv integrin antagonist is presented in an amount greater than about 0.5% w/w; and
(ii) the solubility enhancing agent is presented in an amount of greater than about 5.0% w/w.

Embodiment no. 41. The formulation of any one of embodiments 7-33, wherein:
(i) the solubility enhancing agent is presented in an amount of greater than about 0.5% w/w; and
(ii) the weight ratio between the solubility enhancing agent and the αv integrin antagonist is greater than about 1:1.

Embodiment no. 42. The formulation of any one of embodiments 7-33, wherein:
(i) the solubility enhancing agent is presented in an amount of greater than about 5.0% w/w; and
(ii) the weight ratio between the solubility enhancing agent and the αv integrin antagonist is greater than about 1:1.

Embodiment no. 43. The formulation of any one of embodiments 7-33, wherein:
(i) the solubility enhancing agent is presented in an amount of greater than about 0.5% w/w; and
(ii) the weight ratio between the solubility enhancing agent and the αv integrin antagonist is greater than about 5:1.

Embodiment no. 44. The formulation of any one of embodiments 7-33, wherein:
the αv integrin antagonist is presented in an amount between about 7.0% w/w and about 8.0% w/w; and
the solubility enhancing agent is presented in an amount between about 22.7% w/w and about 23.7% w/w.

Embodiment no. 45. The formulation of any one of embodiments 7-33, wherein:
the αv integrin antagonist is presented in an amount between about 4.2% w/w and about 5.2% w/w; and
the solubility enhancing agent is presented in an amount between about 14.1% w/w and about 15.1% w/w.

Embodiment no. 46. The formulation of any one of embodiments 7-33, wherein:
the αv integrin antagonist is presented in an amount between about 1.9% w/w and about 2.9% w/w; and
the solubility enhancing agent is presented in an amount between about 7.0% w/w and about 8.0% w/w.

Embodiment no. 47. The formulation of any one of embodiments 7-33, wherein:
the αv integrin antagonist is presented in an amount between about 0.5% w/w and about 1.5% w/w; and
the solubility enhancing agent is presented in an amount between about 2.6% w/w and about 3.6% w/w.

Embodiment no. 48. A formulation for topical administration to the eye of a subject in need thereof, comprising:
an αv integrin antagonist having the structure:

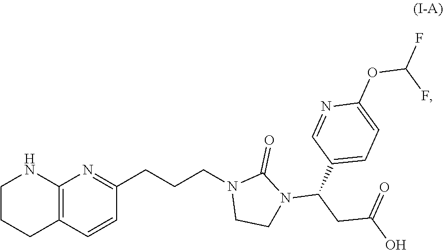 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 7.5% w/w; and
a cyclodextrin in an amount of about 23.25% w/w.

Embodiment no. 49. A formulation for topical administration to the eye of a subject in need thereof, comprising:
an αv integrin antagonist having the structure:

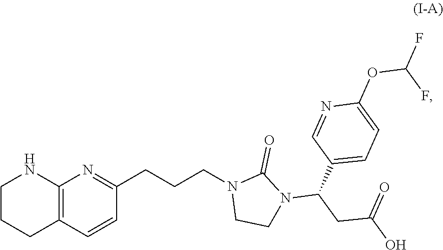 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 7.5% w/w;
a cyclodextrin in an amount of about 23.25% w/w;
benzalkonium chloride in an amount of about 0.02% w/w;
sodium ethylenediaminetetraacetic acid in an amount of about 0.1% w/w; and
a boric acid in an amount of about 1% w/w.

Embodiment no. 50. A formulation for topical administration to the eye of a subject in need thereof, comprising:
an αv integrin antagonist having the structure:

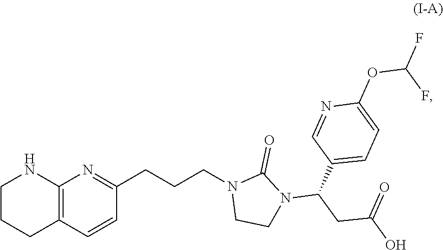 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 4.7% w/w; and
a cyclodextrin in an amount of about 14.6% w/w.

Embodiment no. 51. A formulation for topical administration to the eye of a subject in need thereof, comprising:
an αv integrin antagonist having the structure:

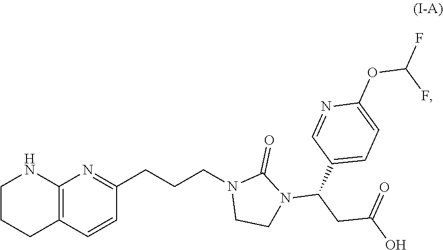 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 4.7% w/w;
a cyclodextrin in an amount of about 14.6% w/w;
benzalkonium chloride in an amount of about 0.02% w/w;
sodium ethylenediaminetetraacetic acid in an amount of about 0.1% w/w; and
a boric acid in an amount of about 1% w/w.

Embodiment no. 52. A formulation for topical administration to the eye of a subject in need thereof, comprising:
an αv integrin antagonist having the structure:

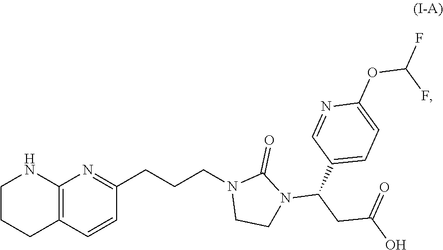 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 2.4% w/w; and
a cyclodextrin in an amount of about 7.5% w/w.

Embodiment no. 53. A formulation for topical administration to the eye of a subject in need thereof, comprising:
an αv integrin antagonist having the structure:

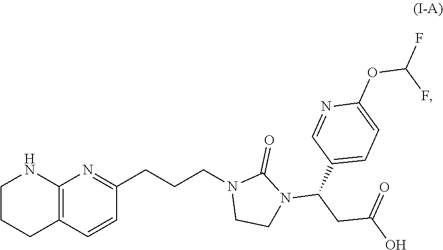 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 2.4% w/w;
a cyclodextrin in an amount of about 7.5% w/w;
benzalkonium chloride in an amount of about 0.02% w/w;
sodium ethylenediaminetetraacetic acid in an amount of about 0.1% w/w; and
a boric acid in an amount of about 1% w/w.

Embodiment no. 54. A formulation for topical administration to the eye of a subject in need thereof, comprising:
an αv integrin antagonist having the structure:

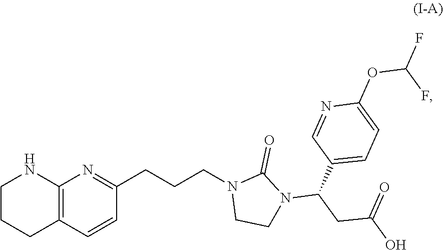 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 1.0% w/w; and
a cyclodextrin in an amount of about 3.1% w/w.

Embodiment no. 55. A formulation for topical administration to the eye of a subject in need thereof, comprising:
an αv integrin antagonist having the structure:

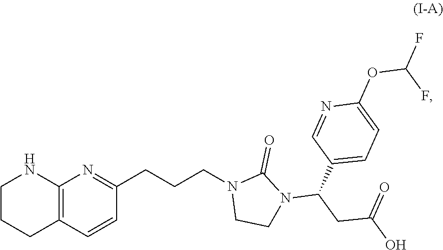 [SMILES: O=C(O)C[C@@H](c1ccc(OC(F)F)nc1)N1CCN(CCCc2ccc3c(n2)NCCC3)C1=O]

or a pharmaceutically acceptable salt or solvate thereof, in an amount of about 1.0% w/w;
a cyclodextrin in an amount of about 3.1% w/w;
benzalkonium chloride in an amount of about 0.02% w/w;
sodium ethylenediaminetetraacetic acid in an amount of about 0.1% w/w; and
a boric acid in an amount of about 1% w/w.

Embodiment no. 56. A method of treating or preventing a disorder mediated by an αv integrin, comprising administering to a subject in need thereof a therapeutically effective amount of the formulation of any one of the preceding embodiments.

Embodiment no. 57. The method of embodiment 56, wherein the αv integrin is an αvβ3 or αvβ5 integrin.

Embodiment no. 58. The method of embodiment 56, wherein the disorder is selected from the group consisting of macular degeneration, diabetic retinopathy (DR), macular edema, diabetic macular edema (DME), and macular edema following retinal vein occlusion (RVO).

Embodiment no. 59. A formulation of any one of embodiments 1-55 for treating or preventing a disorder mediated by an αv integrin.

Embodiment no. 60. A formulation of any one of embodiments 1-55 for use in the manufacture of a medicament for the treatment or prevention a disorder mediated by an αv integrin.

Embodiment no. 61. Use of a formulation of any one of embodiments 1-55 in in the manufacture of a medicament for the treatment or prevention a disorder mediated by an αv integrin.

Embodiment no. 62. An aqueous, ophthalmic solution, comprising:
a. from 4.4% (w/w) to 5.0% (w/w) of a compound of Formula I:

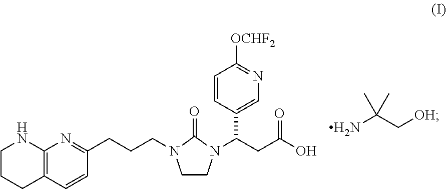

b. from 14% (w/w) to 15% (w/w) of 2-hydroxypropyl-β-cyclodextrin;
c. from 0.5% (w/w) to 1.5% (w/w) of a buffer comprising boric acid;
d. from 0.01% (w/w) to 0.2% (w/w) of a benzalkonium salt; and
e. at least 75% (w/w) water;
wherein the solution has a pH in the range of 7.8 to 8.5.

Embodiment no. 63. An aqueous, ophthalmic solution, comprising:
a. about 4.7% (w/w) of a compound of Formula I:

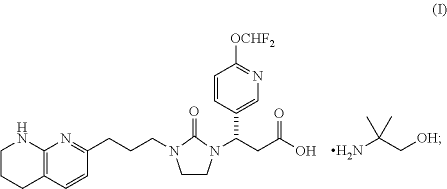

b. about 14.6% (w/w) of 2-hydroxypropyl-β-cyclodextrin;
c. about 1% (w/w) of a buffer comprising boric acid;
d. about 0.02% (w/w) of a benzalkonium salt; and
e. at least 75% (w/w) water;
wherein the solution has a pH in the range of 7.8 to 8.5.

Embodiment no. 64. An aqueous, ophthalmic solution, comprising:
a. 4.7% (w/w) of a compound of Formula I:

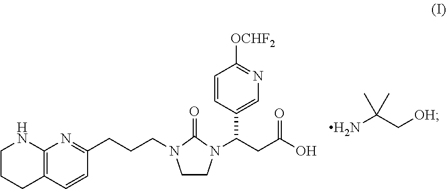

b. 14.6% (w/w) of 2-hydroxypropyl-β-cyclodextrin;
c. 1% (w/w) of a buffer comprising boric acid;
d. 0.02% (w/w) of a benzalkonium salt; and
e. at least 75% (w/w) water;
wherein the solution has a pH in the range of 7.8 to 8.5.

### EXAMPLES

The invention now being generally described, will be more readily understood by reference to the following examples, which are included merely for purposes of illustration of certain aspects and embodiments of the present invention, and are not intended to limit the invention.

### Abbreviations Used in the Following Examples and Elsewhere Herein are

BAC: benzalkonium chloride
HPβCD: hydroxypropyl-β-cyclodextrin
Na-EDTA: sodium ethylenediaminetetraacetic acid or disodium edetate
EDTA: ethylenediaminetetraacetic acid
SWFI: sterile water for injection
PVP: polyvinyl pyrrolidone
PEG: polyethylene glycol
DEXOLVE-7™: sulfobutylether β-cyclodextrin-7, aka SBECD-7
DEXOLVE-4™: sulfobutylether β-cyclodextrin-4, aka SBECD-4.

### Example 1—Preparation of Aqueous Formulation of Compound 1

Aqueous Formulation A containing Compound 1, as set forth in the following table, was prepared. Components in aqueous Formulation A are as set forth in the following table. Aqueous Formulation A had a pH of 8.0. Experimental procedures used to prepare the formulation are described below.

### Aqueous Formulation A

| Component | Amount |
|---|---|
| Compound 1 | 5.0% wt/vol |
| Hydroxypropyl-β-cyclodextrin | 15.5% wt/vol |
| Boric Acid Buffer | 1% wt/vol |
| Sodium ethylene-diamine-tetra- | 0.1% wt/vol |
| acetate | |
| Benzalkonium Chloride | 0.02% wt/vol |
| Hydrochloric Acid (1N in water) | Q.S. to pH 8.0 |
| Potassium Hydroxide (1N in water) | Q.S. to pH 8.0 |
| sterile water for injection | Q.S. |

Compound 1 has the following formula:

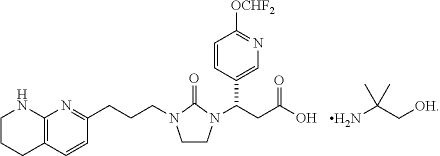

### Preparation Procedures

The following procedure was used to prepare a 12-liter batch of Formulation A:
1. To a tared 20-L stainless-steel vessel, was added sterile water for injection (SWFI) equal to 50% of the target batch size, and stirring was initiated using an overhead mixer.
2. The target amount of hydroxypropyl-beta-cyclodextrin (HPβCD) was weighed and added to the vessel. The mixture was stirred until the HPβCD was dissolved.
3. The target amount of boric acid (powder) was weighed and added to the vessel. The mixture was stirred until the boric acid was dissolved.
4. The target amount of Compound 1 was weighed and added to the vessel. The mixture was blanketed with nitrogen supplied through a 0.20 micron filter and stirred until Compound 1 was dissolved.
5. The target amount of sodium ethylene-diamine-tetra-acetate (in the form of edetate disodium dihydrate) was weighed and added to the vessel. Then, the mixture was stirred until the sodium ethylene-diamine-tetra-acetate was dissolved.
6. Into a beaker fitted with a stir bar was added the target amount of benzalkonium chloride (BAC), NF grade and one half of the remainder (minus circa 5%) of the required sterile water for injection (SWFI). The mixture was stirred until it formed a homogeneous solution.
7. Once the BAC formed a homogeneous solution, the stir bar was removed from the beaker, and the BAC solution was added to the stainless-steel vessel. The BAC beaker was rinsed with the other half of the remainder of the required SWFI, and this SWFI rinse was added to the vessel.
8. A 1N HCL or 1N KOH SWFI solution was titrated into the vessel to adjust the pH of the solution to the target of 8.0 (where permitted pH range is 7.85-8.14).
9. The vessel was weighted, and the weight of the solution was calculated.
10. Additional SWFI was added to bring the final weight of the solution to target amount.
11. The solution was mixed for a minimum of five minutes.
12. Stirring was discontinued and the final weight of the solution was obtained.

Thereafter, the next steps were:
1. The solution was filtered through a course filter (Pall Kleenpak capsule filter with HCD II membrane (1.2 micron) polypropylene (KA1J012P2)). Pressure was monitored to ensure it did not exceed 20 psi.
2. A sample was collected for bioburden testing.
3. The solution was filtered through sterilization 0.2 μm, Kleenpak Fluorodyne® II DFL Membrane filters (2) into a sterile TepoFlex® Biocontainer Assembly, 20 L bag. Pressure was monitored to ensure it did not exceed 20 psi.
4. A bubble point test of both sterilizing filters was conducted.
5. The solution was filled in 3.75-mL portions into pre-sterilized, white opaque low-density polyethylene (LDPE) dropper bottles (Medisize/Adelphi 10 mL (article number 35018W/1 with 0.05 mL LDPE nozzle (Medisize/Adelphi article number 92279) and an HDPE screw cap (Medisize/Adelphi article number 6504.302EP/1) in a Class 100 hood using aseptic technique.

### Example 2—Antimicrobial Analysis for Exemplary Aqueous Formulations of Compound 1 Containing Differing Amounts of Preservative

Twelve aqueous formulations containing hydroxypropyl-β-cyclodextrin (HPβCD), ethylenediaminetetraacetic acid (EDTA), boric acid, benzalkonium chloride (BAC), and in certain instances Compound 1 and/or sodium chloride (NaCl) were prepared and tested for antimicrobial effectiveness. A description of the twelve formulations is provided in the following table.

### Formulations Containing Compound 1

| | | Component Concentrations (% w/w) | | | | | |
|---|---|---|---|---|---|---|---|
| | HPβCD:Compound | Compound | | | Boric | | |
| Formulation | 1 (Molar) | 1 | HPβCD | EDTA | Acid | BAC | NaCl |
| 1 | 1.25:1 | 1.25 | 3.87 | 0.1 | 1 | 0.005 | 0.24 |
| 2 | 1.25:1 | 2.5 | 7.75 | 0.1 | 1 | 0.005 | 0.07 |
| 3 | 1.25:1 | 5.0 | 15.5 | 0.1 | 1 | 0.005 | 0 |
| 4 | n/a | 0 | 15.5 | 0.1 | 1 | 0.005 | 0 |
| 5 | 1.25:1 | 1.25 | 3.87 | 0.1 | 1 | 0.01 | 0.24 |
| 6 | 1.25:1 | 2.5 | 7.75 | 0.1 | 1 | 0.01 | 0.07 |
| 7 | 1.25:1 | 5.0 | 15.5 | 0.1 | 1 | 0.01 | 0 |
| 8 | n/a | 0 | 15.5 | 0.1 | 1 | 0.01 | 0 |
| 9 | 1.25:1 | 1.25 | 3.87 | 0.1 | 1 | 0.02 | 0.24 |
| 10 | 1.25:1 | 2.5 | 7.75 | 0.1 | 1 | 0.02 | 0.07 |
| 11 | 1.25:1 | 5.0 | 15.5 | 0.1 | 1 | 0.02 | 0 |
| 12 | n/a | 0 | 15.5 | 0.1 | 1 | 0.02 | 0 |

Each formulation had a pH of about 8.0. The formulations were prepared based on procedures described in Example 1 above. Formulations were filled in bulk into Type I glass media bottles. After pH and osmolality testing, bulk samples were sent for antimicrobial effectiveness testing (AET). All formulations containing BAC in an amount of 0.02% w/v passed AET (formulations 9-12). In contrast, all formulations containing BAC in an amount of 0.01% w/v or 0.005% w/v failed to pass AET (formulations 1-8). This result demonstrates the benefit of having BAC in an amount of 0.02% w/v in the solution.

Compound 1 has the following formula:

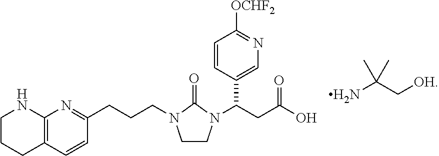

### Example 3—Antimicrobial Analysis for Formulation A from Example 1

Formulation A from Example 1 was evaluated for stability against bacterial growth, yeast growth, and mold growth. Formulation A demonstrated effectiveness in preventing the growth of a microbial challenge in accordance with USP <51> at 7, 14 and 28 days to a standardized inoculum as specified in the compendia method, using USP <61> for microbial enumeration, as follows:
Antimicrobial Effectiveness Testing Bacterial Growth Suppression Effectiveness Requirements:
   NLT a one log reduction from initial calculated count at 7 days;
   NLT a three log reduction from initial calculated count at 14 days; and
   No increase from initial calculated count between 14 and 28 day.
Antimicrobial Effectiveness Testing Yeast and Molds Suppression Effectiveness Requirements:
   No increase from initial count at 7, 14 or 28 days; and
   No increase from initial calculated count at 7, 14 and 28 days.

### Example 4—Evaluating Stability of Exemplary Aqueous Formulations of Compound 1 Containing Different Amounts of Hydroxypropyl-β-cyclodextrin (HPβCD)

The aqueous formulations of Compound 1 in the Tables 1, 2, and 3 were prepared and evaluated for stability to storage at room temperature for a duration of two weeks. Formulations 2 and 3 in Table 1 below were observed to produce a white precipitate during the stability study. The white precipitate was determined to be the following monohydrate compound (hereinafter “Compound B”):

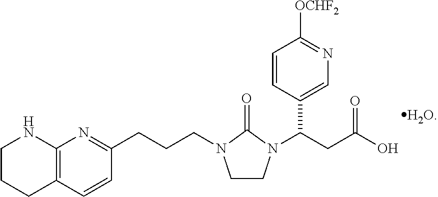 [SMILES: O=C(O)C[C@@H](c1ccc(OC(F)F)nc1)N1CCN(CCCc2ccc3c(n2)NCCC3)C1=O]

The molar ratio of HPβCD to Compound 1 was 1.1:1 for Formulations 2 and 3 in Table 1. In contrast, no white precipitate was observed in Formulations 5 and 6 from Table 1 during the stability study. Formulations 5 and 6 in Table 1 contained a larger amount of HPβCD, i.e., HPβCD in an amount that provides a molar ratio of HPβCD to Compound 1 of 1.25:1. The increased amount of HPβCD in Formulations 5 and 6 in Table 1 improved the stability of Formulations 5 and 6 in Table 1 compared to Formulations 2 and 3 in Table 1.

TABLE 1
AQUEOUS FORMULATIONS OF COMPOUND 1.*

| | Amount of | | Amount of | Amount of Boric | | | Assay, | Precipitate |
|---|---|---|---|---|---|---|---|---|
| | Compound 1 | Mole Ratio | HPβCD | Acid or Other | | Osmolality | 1 week | Observed? |
| No. | (% w/w) | HPβCD:Compound 1 | (% w/w) | Buffer (% w/w) | pH | (mOsm/kg) | (% LC) | (Yes/No) |
| 1 | 2.5 | 1.1:1 | 6.82 | 1 | 8.0 | 267 | 99.4 | No |
| 2 | 5 | 1.1:1 | 13.64 | 1 | 8.0 | 401 | 103.4 | Yes |
| 3 | 7.5 | 1.1:1 | 20.45 | 1 | 8.0 | 628 | 105 | Yes |
| 4 | 2.5 | 1.25:1 | 7.75 | 1 | 8.0 | 279 | 98.6 | No |
| 5 | 5 | 1.25:1 | 15.5 | 1 | 8.0 | 425 | 103.6 | No |
| 6 | 7.5 | 1.25:1 | 23.25 | 1 | 8.0 | 694 | 107.5 | No |

*All formulations contained 0.1% w/w EDTA, 0.02% w/w benzalkonium chloride, and were adjusted to target pH by either HCl or KOH addition.

TABLE 2
AQUEOUS FORMULATIONS OF COMPOUND 1.*

| | Amount of | Mole Ratio | Amount of | Amount of Boric | | | Assay, | Precipitate |
|---|---|---|---|---|---|---|---|---|
| | Compound 1 | HPβCD:Compound | HPβCD | Acid or Other | | Osmolality | 1 week | Observed? |
| No. | (% w/w) | 1 | (% w/w) | Buffer (% w/w) | pH | (mOsm/kg) | (% LC) | (Yes/No) |
| 1 | 2.5 | 2:1 | 12.4 | 1 | 8.0 | 329 | 101.4 | No |
| 2 | 2.5 | 3:1 | 18.6 | 1 | 8.0 | 419 | 102.4 | No |
| 3 | 2.5 | 2:1 | 12.4 | 1 | 8.2 | 331 | 99.7 | No |
| 4 | 2.5 | 3:1 | 18.6 | 1 | 8.2 | 424 | 102.6 | No |
| 5 | 2.5 | 2:1 | 12.4 | 0.49 TRIS | 8.0 | 254 | 99.8 | No |
| 6 | 2.5 | 2:1 | 12.4 | 0.49 TRIS | 8.2 | 246 | 99.7 | No |
| 7 | 2.5 | 2:1 | 12.4 | 0.3 DEA | 8.0 | 236 | 101.2 | No |
| 8 | 2.5 | 2:1 | 12.4 | 0.3 DEA | 8.2 | 224 | 100.9 | No |

*All formulations contained 0.1% w/w EDTA, 0.02% w/w benzalkonium chloride, and were adjusted to target pH by either HCl or KOH addition.

TABLE 3
AQUEOUS FORMULATIONS OF COMPOUND 1.*

| | Amount of | Mole Ratio | Amount of | Amount of Boric | | | Assay, | Precipitate |
|---|---|---|---|---|---|---|---|---|
| | Compound 1 | HPβCD:Compound | HPβCD | Acid or Other | | Osmolality | 1 week | Observed? |
| No. | (% w/w) | 1 | (% w/w) | Buffer (% w/w) | pH | (mOsm/kg) | (% LC) | (Yes/No) |
| 1 | 5 | 2:1 | 24.79 | 1 | 8.0 | 642 | 104.3 | No |
| 2 | 5 | 3:1 | 37.19 | 1 | 8.0 | 1245 | 107.7 | No |
| 3 | 7.5 | 2:1 | 37.19 | 1 | 8.0 | 1432 | 107.4 | No |

*All formulations contained 0.1% w/w EDTA, 0.02% w/w benzalkonium chloride, and were adjusted to target pH by either HCl or KOH addition.

### Example 5—Preparation of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid Monohydrate

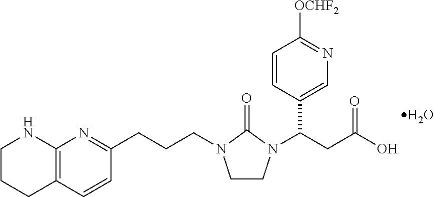

The title compound was prepared according to the following procedures.

### Part 1—Preparation of tert-butyl (S)-3-(6-(difluoromethoxy)pyridin-3-yl)-3-(1-(2,2-dimethoxyethyl)-3-(3-(5,6,7,8-tetrahydro-1,8-naphthyridin-2-yl)propyl)ureido)propanoate

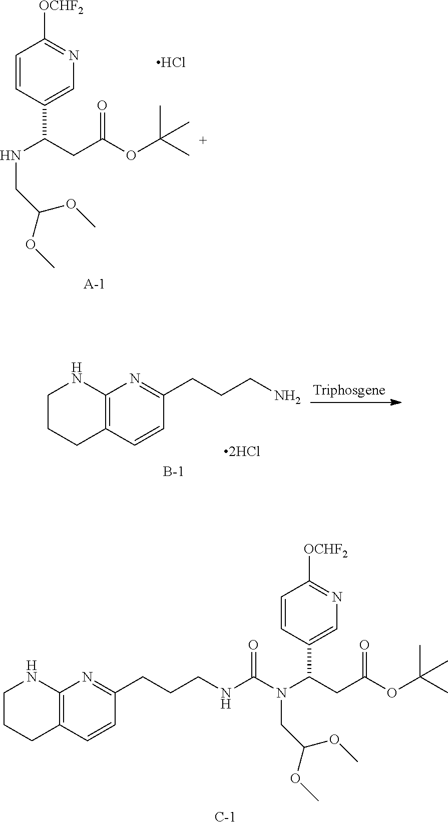

Compound A-1 (1.0 eq.) was mixed with amine B-1 (1.2 eq.), Et₃N (10 eq.), and triphosgene (0.8 eq.) in dichloromethane and the reaction mixture was held at 25° C. to 30° C. for 5 hours. Thereafter, the reaction mixture was cooled to 0-5° C. and then quenched with water. The organic layer from the resulting mixture was isolated and then washed with 5% aq. NaHCO₃ solution followed by a water wash and then concentrated to provide the title compound (reaction yield was 76%).

### Part 2—Preparation of (S)-3-(6-(difluoromethoxy)pyridin-3-yl)-3-(2-oxo-3-(3-(5,6,7,8-tetrahydro-1,8-naphthyridin-2-yl)propyl)-2,3-dihydro-1H-imidazol-1-yl)propanoic Acid

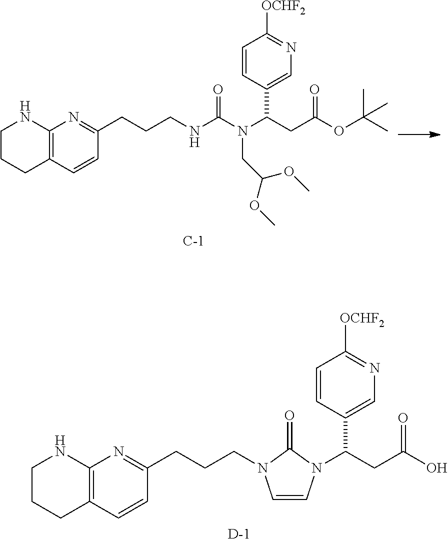

Compound C-1 (1.0 eq.) was treated with concentrated aqueous H₂SO₄ (2M) in tetrahydrofuran and held at a temperature of 25° C. to 30° C. for a duration of 3 hours. Then, the resultant ester compound (1.0 eq.) was treated with concentrated aqueous H₂SO₄ (3.5M) in tetrahydrofuran and held at a temperature of 50-55° C. for 12 hours. Once the reaction was complete, the title compound was isolated from the reaction mixture and purified by column chromatography. Reaction yield was 70%.

### Part 3—Preparation of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid

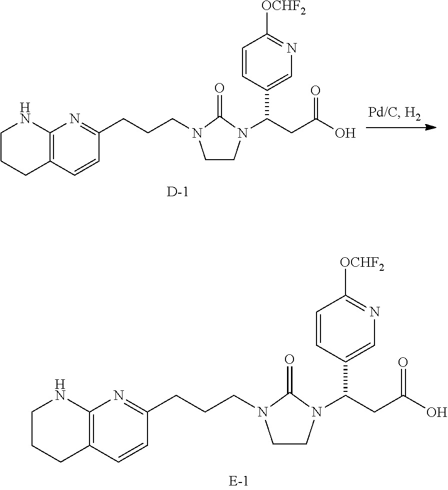

Compound D-1 (1.0 eq.) was treated with 10% Pd/C (20% w/w), methanol, NH₃ (5 kg/cm2), and hydrogen gas at a temperature of 50° C. for a duration of 62-65 hours. Then, the reaction mixture was filtered, and solvent was evaporated from the filtrate to provide the title compound. Reaction yield was 73%.

### Part 4—Preparation of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid Monohydrate

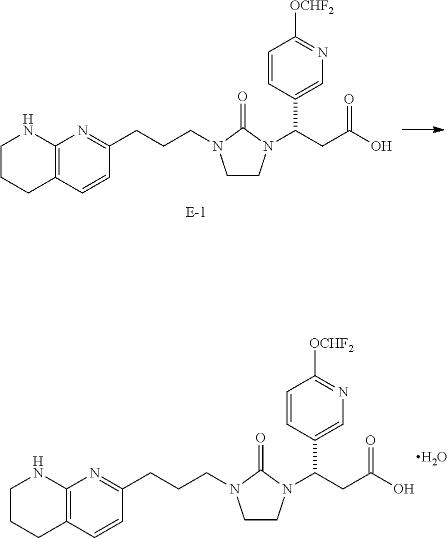

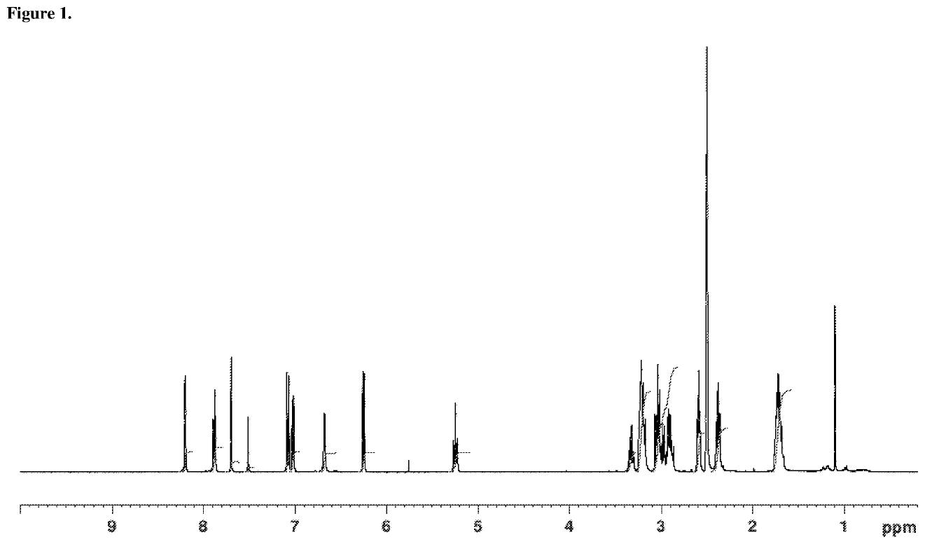

Compound E-1 was dissolved in 5% w/w potassium carbonate solution. The resulting solution was washed with methyl tert-butyl ether (MTBE), and the aqueous layer was adjusted to a pH in the range of 7.0 to 7.5 by the addition of an aqueous 5% w/w formic acid solution. Then, the resulting mixture was extracted with dichloromethane. The organic layer was dried over sodium sulphate and concentrated under reduced pressure to provide crude product. The crude product was stirred with n-heptane, filtered, and dried to provide the title compound. The title compound had a water content of 3.6% by weight. The compound was characterized by ¹H NMR spectroscopy (400 MHz) in deuterated DMSO. The ¹H NMR spectrum is provided in FIG. 1.

### Example 6—Alternative Preparation of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid Monohydrate

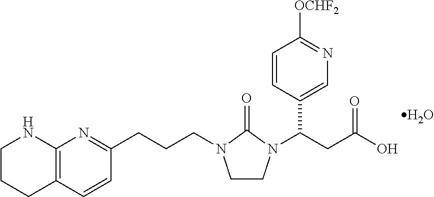

The title compound was prepared according to the following procedure.

### Part 1—Preparation of 2-amino-2-methylpropan-1-ol (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid

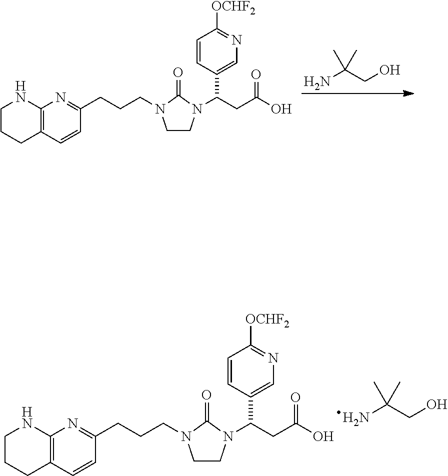

(S)-3-(6-(Difluoromethoxy)pyridin-3-yl)-3-(2-oxo-3-(3-(5,6,7,8-tetrahydro-1,8-naphthyridin-2-yl)propyl)imidazolidin-1-yl)propanoic acid (1 eq.) and 2-amino-2-methylpropan-1-ol (2 eq.) were combined with acetonitrile and held at 25° C. to 30° C., which resulted in the formulation of a white solid. Thereafter, the white solid was collected by filtration under argon, and the collected white solid was dried.

### Part 2—(S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid Monohydrate

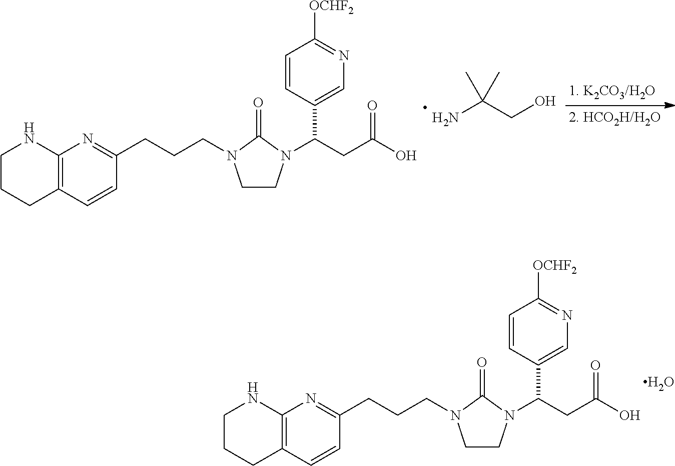

The compound 2-amino-2-methylpropan-1-ol (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid was dissolved in a 5% w/w aqueous potassium carbonate solution. The resulting solution was washed with methyl tert-butyl ether. Then, the aqueous layer was adjusted to a pH in the range of 7.0 to 7.5 by the addition of an aqueous 5% w/w formic acid solution. Then, the resulting mixture was extracted with dichloromethane. The organic layer was dried over sodium sulphate and concentrated under reduced pressure to provide crude product. The crude product was stirred with n-heptane, filtered, and dried to provide the title compound. The title compound produced by this procedure had a water content of 3.6%. The compound was characterized by difference thermogravimetry (DTG). DTG analysis identified a weight loss of approximately 3.86% at a temperature in the range of 25-30° C. Accordingly, the title compound is a monohydrate.

### Example 7—Characterization of Crystalline (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic Acid Monohydrate

A sample of crystalline (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate was analyzed by X-ray crystallography. The crystalline (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate was determined to have an orthorhombic crystal system, with a P2₁2₁2 space group. Experimental details of the X-ray crystallographic analysis are provided below.

A crystal of (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate was mounted on a diffractometer and X-ray data was collected at a temperature of 100 K. The intensities of the reflections were collected by means of a Bruker APEX DUO CCD diffractometer (Cu_{Kα} radiation, λ=1.54178 Å), and equipped with an Oxford Cryosystems nitrogen flow apparatus. The collection method involved 1.0° scans in ω at −30°, −55°, −80°, 30°, 55°, 80° and 115° in 2θ. Data integration down to 0.84 Å resolution was carried out using SAINT V8.37 A (Bruker diffractometer, 2015) with reflection spot size optimization. Absorption corrections were made with the program SADABS (Bruker diffractometer, 2015). The structure was solved by the Intrinsic Phasing methods and refined by least-squares methods again F² using SHELXT-2014 (G. M. Sheldrick, Acta Cryst. 2015. A71, 3-8) and SHELXL-2014 (G. M. Sheldrick, Acta Cryst. 2015, C71, 3-8) with OLEX 2 interface (Dolomanov et al. in J. Appl. Cryst. 2009, vol. 42, 339-341). Non-hydrogen atoms were refined anisotropically, and hydrogen atoms were allowed to ride on the respective atoms. Crystal data as well as details of data collection and refinement are summarized in the table below.

Crystal data

| Chemical formula | C₂₃H₂₉F₂N₅O₅ |
|---|---|
| Mᵣ | 493.47 |
| Crystal system, space group | Orthorhombic, P2₁2₁2 |
| Temperature (K) | 100 |
| a, b, c (Å) | 18.1127 (7), 19.6258 (8), 12.9599 (5) |
| V (Å³) | 4606.9 (3) |
| Z | 8 |
| Radiation type | Cu Kα |
| μ (mm⁻¹) | 0.95 |
| Crystal size (mm) | 0.18 × 0.12 × 0.10 |

Data collection

| | |
|---|---|
| Diffractometer | Bruker D8 goniometer with CCD area detector |
| Absorption correction | Multi-scan |
| | SADABS |
| Tₘᵢₙ, Tₘₐₓ | 0.807, 0.864 |
| No. of measured, independent | 81855, 8147, 7976 |
| and observed [I > 2σ(I)] | |
| reflections | |
| Rᵢₙₜ | 0.040 |
| (sin θ/λ)ₘₐₓ (Å⁻¹) | 0.596 |

Refinement

| | |
|---|---|
| R[F² > 2σ(F²)], wR(F²), S | 0.039, 0.108, 1.08 |
| No. of reflections | 8147 |
| No. of parameters | 696 |
| No. of restraints | 32 |
| H-atom treatment | H atoms treated by a mixture of independent and |
| | constrained refinement |
| Δρₘₐₓ, Δρₘᵢₙ (e Å⁻³) | 0.30, −0.29 |
| Absolute structure | Flack x determined using 3421 quotients |
| | [(I+) − (I−)]/[(I+) + (I−)] (Parsons, Flack and Wagner, Acta Cryst. |
| | B69 (2013) 249-259). |
| Absolute structure parameter | −0.01 (4) |

### Example 8—Osmolality Analysis of Exemplary Aqueous Formulations of Compound 1

Four formulations containing different amounts of Compound 1, HPβCD, and sodium chloride (NaCl) were prepared and tested for osmolality. The four formulations were prepared with the components/constraints listed in Table 1 below, according to the procedure set forth below.

All compounding, filtering, and filling into containers was conducted on an open benchtop. To sterile water for injection (SWFI) were added EDTA, HPβCD, and boric acid, and the mixture was allowed 15 minutes for complete dissolution. Then, sodium chloride (NaCl) and compound 1 were added to mixture, and the resulting mixture was allowed one hour for complete dissolution. Next, benzalkonium chloride (BAC) was added. The pH of the mixture was adjusted using 1N potassium hydroxide (KOH) or 1N hydrochloric acid (HCl). After the final q.s. with SWFI, each formulation was passed through 1.2 μm and 0.2 μm filters in series before being dispensed. Each formulation was dispensed into two translucent eye dropper bottles and two filled clear glass vials (to facilitate observation of formed solids).

TABLE 1
COMPOSITIONS AND OSMOLALITY FOR ADDITIONAL
FORMULATIONS OF COMPOUND 1

| | | | Component Concentrations (% w/w) | | | | | | |
|---|---|---|---|---|---|---|---|---|---|
| | | HPβCD:Compound | Compound | | | Boric | | | Osmolality |
| No. | pH | 1 (mole ratio) | 1 | HPβCD | EDTA | Acid | BAC | NaCl | (mOsm/kg) |
| 1 | 8.0 | 1.25:1 | 1.25 | 3.87 | 0.1 | 1 | 0.02 | 0.19 | 283.2 |
| 2 | 8.0 | 1.25:1 | 2.5 | 7.75 | 0.1 | 1 | 0.02 | 0.07 | 303.2 |
| 3 | 8.0 | 1.25:1 | 5.0 | 15.5 | 0.1 | 1 | 0.02 | 0 | 430.6 |
| 4 | 8.0 | n/a | 0 | 15.5 | 0.1 | 1 | 0.02 | 0 | 326.6 |

### Example 9—Preparation of Additional Exemplary Aqueous Formulations of Compound 1

Three formulations containing Compound 1 were prepared and evaluated as set forth below. Compound 1 has the following formula:

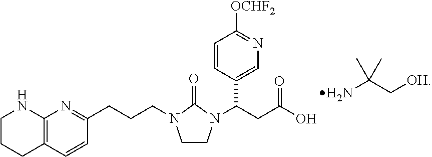

Each formulation (i.e., Formulation 1, Formulation 2, and Formulation 3) was prepared in the amount of 2 mL in volume and then tested to evaluate physical stability. Tests for physical stability of the formulations were conducted at room temperature for 72 hr.

### Preparation of Formulation 1

Boric acid (20 mg), PEG 6000 (20 mg), and HPβCD (100 mg) were dissolved in a Compound 1 stock solution (aq., 200 mg/mL, 1.5 mL). Na-EDTA (20 mg/mL, 100 μL) and BAC (aq., 2 mg/mL, 100 μL) were added and homogenized by stirring. The final pH was adjusted to 8.0 by adding HCl (aq., 1N). The solution was brought to the desired volume of 2.0 mL with purified water.

### Preparation of Formulation 2

Sodium citrate (20 mg), PVP (20 mg), and Dexolve-7 (60 mg) were dissolved in a Compound 1 stock solution (aq., 200 mg/mL, 1.5 mL). Na-EDTA (20 mg/mL, 100 μL) and BAC (aq., 2 mg/mL, 100 μL) were added and homogenized by stirring. The final pH was adjusted to 7.9 by adding HCl (aq., 1N). The solution was brought to the desired volume of 2.0 mL with purified water.

### Preparation of Formulation 3

Sodium citrate (20 mg), PEG 6000 (20 mg), Dexolve-4 (60 mg) were dissolved in a Compound 1 stock solution (aq., 200 mg/mL, 1.5 mL). Na-EDTA (20 mg/mL, 100 L) and BAC (aq., 2 mg/mL, 100 μL) were added and solution was homogenized by stirring. The final pH was adjusted to 7.9 by adding HCl (aq., 1N). The solution was brought to the desired volume of 2.0 mL with purified water.

After being stored at room temperature for 72 hours, none of the above formulations suffered any visible change.

The solubilizing capacity of Formulations 1-3 was tested by adding solid Compound 1 into each of the formulations in 10 mg steps, under vigorous stirring. Each step resulted in a 5 mg/mL increase of the final concentration. No limits were found for the solubilizing potencies of the formulations up to 200 mg/mL Compound 1.

Addition of Compound 1 resulted in pH values of 8.50, 8.56, and 8.42 for Formulations 1, 2, and 3, respectively. Addition of HCl (1N) reduced the pH to the target level without causing precipitation. All formulations were filtered through a 0.22 μm pore size membrane and sterile filtered without difficulty.

### Example 10—Preparation of Additional Aqueous Formulations of Compound 1

Engineering (non-GMP) batches including 0.005% w/w benzalkonium chloride (BAC) were prepared using Compound 1 at a concentration of 0.5%, 2.5%, or 5.0% wt/wt. The formulations described in Table 1 were prepared in batches of 2,000 g each. The batches underwent comprehensive mock release (non-GMP) testing and were placed on supportive stability. The results are summarized in Table 2. All three engineering batches met nearly all GMP release testing acceptance criteria at the time of mock release testing, except the formulation comprising 0.5% w/w Compound 1, which failed to meet USP <51> requirements for growth suppression upon microbial challenge.

TABLE 1
OPHTHALMIC FORMULATIONS (ENGINEERING BATCH) INCORPORATING 0.005% W/W BAC

| | | 0.5% Formulation | | 2.5% Formulation | | 5.0% Formulation | |
|---|---|---|---|---|---|---|---|
| | Compendia | Final | Amount | Final | Amount | Final | Amount |
| Component | Grade | Conc. (w/w) | (g) | Conc. (w/w) | (g) | Conc. (w/w) | (g) |
| Compound 1 | NA | 0.5% | 10.0 | 2.5% | 50.01 | 5.0% | 100 |
| BAC (1 mg/g, | NF^{a} | 0.005% | 100 | 0.005% | 100 | 0.005% | 100 |
| aq.) | | | | | | | |
| HPβCD | NF | 5% | 100.01 | 5% | 100 | 5% | 100 |
| Boric Acid | NF | 1% | 20 | 1% | 20 | 1% | 20 |
| Na-EDTA (20 | USP | 0.10% | 100 | 0.10% | 100 | 0.10% | 100 |
| mg/g, aq.) | | | | | | | |
| HCl (1N) | NF | To adjust | | To adjust | | To adjust | |
| | | to pH 8.0 | | to pH 8.0 | | to pH 8.0 | |
| NaOH (1N) | NF | To adjust | 49.47 | To adjust | | To adjust | |
| | | to pH 8.0 | | to pH 8.0 | | to pH 8.0 | |
| SWFI | USP | q.s to 100% | 1620.53 | q.s to 100% | 1630.3 | q.s to 100% | 1580 |

NA = not applicable;
NF = National Formulary;
USP = United States Pharmacopeia;
— = not used
^{a} = Compendia listed as NF in USP 39/NF 34.
Certificate of analysis from Sigma Aldrich lists as USP grade.

TABLE 2
ANALYSIS OF FORMULATIONS (ENGINEERING BATCH) INCORPORATING 0.005% W/W BAC

| Test | Test | GMP Acceptance | Results | | |
|---|---|---|---|---|---|
| Parameter | Method | Criteria^{d} | 0.5% Formulation | 2.5% Formulation | 5.0% Formulation |
| Compound 1 | PSMET# | 90-110% | 101% | 102% | 100% |
| (% of Label | AS0143^{a} | | | | |
| Claim) | (draft) | | | | |
| Related | PSMET# | Individual | RRT 0.79 = 0.05% | RRT 0.79 = 0.05% | RRT 0.79 = 0.05% |
| Substances | AS0143 | impurities: ≤1.0% | RRT 0.95 = 0.11% | RRT 0.95 = 0.09% | RRT 0.95 = 0.09% |
| (% of Label | (draft) | Report for Each ≥ | RRT 0.96 = 0.21% | RRT 0.96 = 0.18% | RRT 0.96 = 0.17% |
| Claim) | | 0.05% | RRT 1.19 = 0.11% | RRT 1.19 =0.11% | RRT 1.19 = 0.11% |
| | | Total | | | RRT 1.24 = 0.05% |
| | | impurities: ≤3.0% | | | |
| Appearance | PSMET# | Light brown solution | Clear, off-brown | Clear, light brown | Clear, light brown |
| | AS0090 | | solution | solution | solution |
| pH | PSSOP# | 7.0-8.5 | 8.1 | 7.8 | 8.1 |
| | 30019 | | | | |
| Osmolality | PSSOP# | Report Results | 212 | 241 | 290 |
| (mOsm/kg) | 30012 | | | | |
| BAC Assay | PSMET# | 50-120% | 91% | 95% | 90% |
| (% of Label | AS0144 | | | | |
| Claim) | | | | | |
| Antimicrobial | USP | Meets specified | Fail^{e} | Pass^{e} | Pass^{e} |
| Effectiveness | <51> | acceptance criteria^{b} | | | |
| Testing (AET) | | | | | |
| Sterility | USP | No Growth | Pass | Pass | Pass |
| | <71> | | | | |
| Bacterial | USP | Report Results | <0.5 EU/mL | <0.5 EU/mL | <1.0 EU/mL |
| Endotoxins | <85> | | | | |
| Particulate | USP | Meets Criteria^{c} | Pass | Pass | Pass |
| Matter in | <789> | | | | |
| Ophthalmic | | | | | |
| Products | | | | | |

^{a}= PSMET #AS0143 (Draft) LOQ = 0.05%
^{b}= Bacterial Growth Suppression Effectiveness (NLT a one log reduction from initial calculated count at 7 days; NLT a three log reduction from initial calculated count at 14 days and no increase from initial calculated count between 14 and 28 day). Yeast and Molds Suppression Effectiveness (No increase from initial count at 7, 14 or 28 days).
^{c}= >10 μm − NMT 50/mL; ≥25 μm − NMT 5/mL; ≥50 μm − NMT 2/mL
^{d}= acceptance criteria apply only to GMP batches but were utilized for non-GMP engineering batches to characterize and assess acceptance criteria suitability
^{e}= results of antimicrobial effectiveness testing in accordance with USP <51> indicated that the Compound 1 2.5% w/w and 5.0% w/w formulation passed acceptance criteria at 7, 14 and 28 days, Compound 1 at 0.5% w/w formulation failed at day 7 of testing.
RRT = Relative Retention Time

For the formulation comprising 0.5% w/w of Compound 1, the BAC concentration was increased to 0.02% w/w. Engineering batches were prepared at the higher antimicrobial level, underwent comprehensive mock release (non-GMP) testing, and were placed on supportive stability. Batches of the formulations comprising 0.02% w/w BAC (2,000 g each) were prepared as described in Table 3. Results of mock release testing are summarized in Table 4. All three engineering batches comprising 0.02% w/w BAC met the GMP release testing acceptance criteria.

TABLE 3
OPHTHALMIC FORMULATIONS INCORPORATING 0.02% W/W BAC

| | | 1.25% Formulation | | 2.5% Formulation | | 5.0% Formulation | |
|---|---|---|---|---|---|---|---|
| | Compendia | Final Conc. | Amount | Final Conc. | Amount | Final Conc. | Amount |
| Component | Grade | (w/w) | (g) | (w/w) | (g) | (w/w) | (g) |
| Compound 1 | NA | 1.25% | 25.0 | 2.5% | 50.00 | 5.0% | 100.00 |
| BAC | NF^{a} | 0.02% | 0.40 | 0.02% | 0.40 | 0.02% | 0.40 |
| HPβCD | NF | 5% | 100.02 | 5% | 100.01 | 5% | 100.00 |
| Boric Acid | NF | 1% | 20.01 | 1% | 20.00 | 1% | 20.00 |
| Na-EDTA | USP | 0.10% | 2.00 | 0.10% | 2.00 | 0.10% | 2.00 |
| HCl (1N) | NF | To adjust | — | To adjust | — | To adjust | 10.00 |
| | | to pH 8.0 | | to pH 8.0 | | to pH 8.0 | |
| NaOH (1N) | NF | To adjust | 44.09 | To adjust | 21.27 | To adjust | — |
| | | to pH 8.0 | | to pH 8.0 | | to pH 8.0 | |
| SWFI | USP | q.s to 100% | 1805.50 | q.s to 100% | 1817.28 | q.s to 100% | 1785.31 |

NA = not applicable;
NF = National Formulary;
USP = United States Pharmacopeia;
— = not used
^{a} = Compendia listed as NF in USP 39/NF 34.
Certificate of analysis from Sigma Aldrich lists as USP grade.

TABLE 4
ANALYSIS OF FORMULATIONS INCORPORATING 0.02% W/W BAC

| Test | Test | GMP Acceptance | Results | | |
|---|---|---|---|---|---|
| Parameter | Method | Criteria^{d} | 1.25% Formulation | 2.5% Formulation | 5.0% Formulation |
| Compound 1 | PSMET# | 90-110% | 102% | 102% | 102% |
| (% of Label | AS0143^{a} | | | | |
| Claim) | (draft) | | | | |
| Related | PSMET# | Individual | RRT 0.80 = 0.05% | RRT 0.80 = 0.05% | RRT 0.80 = 0.05% |
| Substances | AS0143 | impurities: ≤1.0% | RRT 0.95 = 0.12% | RRT 0.95 = 0.12% | RRT 0.95 = 0.12% |
| (% of Label | (draft) | Report for Each ≥ | RRT 0.96 = 0.21% | RRT 0.96 = 0.20% | RRT 0.96 = 0.19% |
| Claim) | | 0.05% | RRT 1.19 = 0.11% | RRT 1.19 = 0.11% | RRT 1.19 = 0.11% |
| | | Total | Total Impurities = | Total Impurities = | Total Impurities = |
| | | impurities: ≤3.0% | 0.49% | 0.48% | 0.47% |
| Appearance | PSMET# | Light brown | Clear light brown | Clear light brown | Clear brownish |
| | AS0090 | solution | solution | solution | solution |
| pH | PSSOP# | 7.0-8.5 | 8.1 | 8.0 | 8.0 |
| | 30019 | | | | |
| Osmolality | PSSOP# | Report Results | 221 | 245 | 300 |
| (mOsm/kg) | 30012 | | | | |
| BAC Assay | PSMET# | 50-120% | 91% | 92% | 93% |
| (% of Label | AS0144 | | | | |
| Claim) | (draft) | | | | |
| Antimicrobial | USP <51> | Meets specified | Pass | Pass | Pass |
| Effectiveness | | acceptance criteria^{b} | | | |
| Testing | | | | | |
| (AET) | | | | | |
| Sterility | USP <71> | No Growth | Pass | Pass | Pass |
| Particulate | USP | Meets Criteria^{c} | Pass | Pass | Pass |
| Matter in | <789> | | | | |
| Ophthalmic | | | | | |
| Products | | | | | |

ND = not determined
^{a}= PSMET #AS0143 (Draft) LOQ = 0.05%
^{b}= Bacterial Growth Suppression Effectiveness (NLT a one log reduction from initial calculated count at 7 days; NLT a three log reduction from initial calculated count at 14 days; and no increase from initial calculated count between 14 and 28 day). Yeast and Molds Suppression Effectiveness (No increase from initial count at 7, 14 or 28 days).
^{c}= ≥10 μm − NMT 50/mL; ≥25 μm − NMT 5/mL; ≥50 μm − NMT 2/mL
^{d}= acceptance criteria apply only to GMP batches but were utilized for non-GMP engineering batches to characterize and assess acceptance criteria suitability
RRT = Relative Retention Time

No overages were incorporated into the formulations. The formulations of Compound 1 at a concentration of 2.5% w/w or 5.0% w/w were prepared to possess an osmolality of approximately 200-300 mOsm/kg. The pH of the formulation was controlled using a 1.0% wt/wt boric acid buffer. HCl or NaH were used to adjust pH of the formulation during compounding to achieve a pH within the range of 7.0-8.5. Particulate levels evaluated on release and stability met USP <789> requirements for ophthalmic solution formulations.

### Example 11—Preparation of Additional Aqueous Formulations Containing Compound 1

The formulations in tables 1-3 below were prepared.

TABLE 1

| | | 2.5% (wt/wt) | 5.0% (wt/wt) |
|---|---|---|---|
| | | Formulation | Formulation |
| | Compendia | Amount of Component | Amount of Component |
| Ingredient | Grade | (wt/wt) | (wt/wt) |
| Compound 1 | NA | 2.5% | 5.0% |
| BAC | NF^{b} | 0.02% | 0.02% |
| HPβCD | EP | 2.5% | 5.0% |
| Boric Acid | NF | 1.0% | 1.0% |
| Na-EDTA | USP | 0.10% | 0.10% |

| | | |
|---|---|---|
| HCl (1N) | NF | To adjust solution to pH 8.0 |
| NaOH (1N) | NF | To adjust solution to pH 8.0 |
| Water for injection | USP | q.s. to 100% |

NA = not applicable;
NF = National Formulary;
USP = United States Pharmacopeia;
EP = European Pharmacopeia
^{a} = Mohamed, M.A.H. et al., Curr. Eye Res. 36, 208, 2011, with rabbit Draize analysis for tolerability.
^{b}= Compendia listed as NF in USP 39/NF 34. Certificate of analysis from Sigma Aldrich lists as USP grade.

TABLE 2

| | | | Target Amount (g) of Component per Batch | |
|---|---|---|---|---|
| | Quality | | Compound 1 | Compound 1 |
| Component | Standard | Conc. (% wt/wt) | (2.5% wt/wt) | (5.0% wt/wt) |
| Compound 1 | In-House | 2.5 or 5.0 | 112.5 | 225.0 |
| | Standard | | | |
| BAC | NF^{a} | 0.02 | 0.9 | 0.9 |
| HPβCD | EP | 5.0 | 225 | 225 |
| Boric Acid | NF | 1.0 | 45 | 45 |
| Na-EDTA | USP | 0.10 | 4.5 | 4.5 |
| HCl (IN) | NF | To adjust to pH 8.0 | Q.S. to pH 8.0 | Q.S. to pH 8.0 |
| KOH (IN) | NF | To adjust to pH 8.0 | Q.S. to pH 8.0 | Q.S. to pH 8.0 |
| SWFI | USP | q.s. to 100% | q.s. | q.s. |
| Total Batch Size | | | 4,500 g | 4,500 g |

NF = National Formulary;
Q.S = sufficient quantity;
USP = United States Pharmacopeia
^{a}= Compendia listed as NF in USP 39/NF 34. Certificate of analysis from Sigma Aldrich lists as USP
grade.

TABLE 3

| | Formulation 1 | Formulation 2 | Formulation 3 | Formulation 4 |
|---|---|---|---|---|
| | Component Conc. | Component Conc. | Component | Component |
| Component | (% wt/wt) | (% wt/wt) | Conc. (% wt/wt) | Conc. (% wt/wt) |
| Compound 1 | 1.0 | 2.4 | 4.7 | 7.5 |
| HPβCD | 3.06 | 7.5 | 14.6 | 23.25 |
| Na-EDTA | 0.1 | 0.1 | 0.1 | 0.1 |
| BAC | 0.02 | 0.02 | 0.02 | 0.02 |
| Boric Acid | 1.0 | 1 | 1 | 1 |
| NaCl | 0.25 | 0.07 | — | — |
| NaOH (1N) | 0.15% | 0.09 | — | — |
| | (for pH adjustment) | (for pH adjustment) | | |
| SWFI | q.s. to 100% | q.s. to 100% | q.s. to 100% | q.s. to 100% |

### Example 12—Excipient Compatibility with Compound 1

Excipients studied for compatibility in the formulations described herein included BAC (benzoxonium chloride), cyclodextrins (e.g., HPβCD, Dexolve-7™, and Dexolve-4™), boric acid and citrate, EDTA, and PVP or PEG 6000 (viscosity enhancer). HCl and NaOH were used for pH adjustment. No incompatibility was found among the excipients and reagents tested. Both boric acid and citrate buffer systems were physically compatible.

### Example 13—Analysis of Ocular Uptake and Ocular Tissue Distribution of Compound 1 in Dutch Belted Rabbits after Topical Ocular Administration

Male Dutch belted rabbits were subjected to topical ocular administration of two formulations of Compound 1 (5.0%). An aliquot (50 μL) of the formulation was administered in each eye according to the dosing scheme presented in Table 1 below.

TABLE 1

| | | | | | | Sample | |
|---|---|---|---|---|---|---|---|
| | Dose | | Dose | Dose | Dose vol. | collection | |
| Group | route | N= | (mg/eye) | conc. | (μL/eye) | time | Samples collected |
| Formulation A | Ocular | 24 (3 | 2.5 | 5.0% | 50 | 1 hr, 4 hr, | Plasma, aqueous humor, vitreous |
| (with PEG | (topical, | per time | | | | 8 hr, & 12 | humor, cornea, conjunctivas, lens, |
| 6000) | each eye) | point) | | | | hr | iris/ciliary body, sclera, and retina- |
| | | | | | | | choroid-plexus |
| Formulation B | Ocular | 24 (3 | 2.5 | 5.0% | 50 | 1 hr, 4 hr, | Plasma, aqueous humor, vitreous |
| (w/o. PEG | (topical, | per time | | | | 8 hr, & 12 | humor, cornea, conjunctivas, lens, |
| 6000) | each eye) | point) | | | | hr | iris/ciliary body, sclera, and retina- |
| | | | | | | | choroid-plexus |

The only difference between Formulation A and Formulation B is that Formulation B did not contain PEG 6000. All other excipients were common to the two formulations: boric acid, HPβCD, Na-EDTA, and BAC. The study allowed a direct comparison between the formulation used in toxicity studies.

The concentration of Compound 1 in the plasma and the ocular tissues after the single dose administration of Formulation A or B are shown in Tables 2 and Table 3, respectively.

TABLE 2
COMPOUND 1 LEVELS IN PLASMA (NG/ML)

Time Point (hour)

| Group | Statistic | 1 | 4 | 8 | 12 |
|---|---|---|---|---|---|
| Group 1 | Average | 174 | 33.8 | 17.8 | 5.21 |
| Formulation A | SD | 46 | 15 | 17 | 1.8 |
| (n = 12) | % RSD | 27 | 43 | 94 | 34 |
| Group 2 | Average | 269 | 41.3 | 13.0 | 6.87 |
| Formulation B | SD | 175 | 20 | 1.0 | 1.1 |
| (n = 12) | % RSD | 65 | 48 | 8.0 | 17 |

TABLE 3
COMPOUND 1 LEVELS IN OCULAR TISSUE

| | | Time Point (hour) | | | | | | | |
|---|---|---|---|---|---|---|---|---|---|
| | | 1 | | 4 | | 8 | | 12 | |
| Group | Statistic | L | R | L | R | L | R | L | R |

Aqueous Humor (ng/mL)

| | | | | | | | | | |
|---|---|---|---|---|---|---|---|---|---|
| Group 1 Male | Average | 1255 | 1224 | 263 | 370 | 34.8 | 77.4 | 9.74 | 32.0 |
| Formulation A | SD | 507 | 466 | 95 | 270 | 7.6 | 78 | 1.8 | 25 |
| (n = 12) | % RSD | 40 | 38 | 36 | 73 | 22 | 100 | 18 | 77 |
| Group 2 Male | Average | 1857 | 2210 | 212 | 234 | 53.7 | 74.2 | 10.0 | 14.0 |
| Formulation B | SD | 1024 | 391 | 39 | 95 | 18 | 32 | 1.1 | 3.5 |
| (n = 12) | % RSD | 55 | 18 | 18 | 41 | 33 | 43 | 11 | 25 |

Vitreous Humor (ng/mL)

| | | | | | | | | | |
|---|---|---|---|---|---|---|---|---|---|
| Group 1 Male | Average | 112 | 210 | 48.1 | 114 | 85.8 | 64.4 | 51.1 | 42.6 |
| Formulation A | SD | 65 | 69 | 30 | 71 | 99 | 77 | 8.8 | 43 |
| (n = 12) | % RSD | 58 | 33 | 62 | 62 | 116 | 119 | 17 | 101 |
| Group 2 Male | Average | 138 | 557 | 89.7 | 116 | 17.9 | 50.8 | 21.2 | 83.4 |
| Formulation B | SD | 60 | 386 | 70 | 82 | 13 | 50 | 13 | 49 |
| (n = 12) | % RSD | 44 | 69 | 78 | 70 | 73 | 99 | 63 | 59 |

Iris-Ciliary Body (ng/g)

| | | | | | | | | | |
|---|---|---|---|---|---|---|---|---|---|
| Group 1 Male | Average | 3765 | 6253 | 1747 | 2068 | 5232 | 1185 | 510 | 927 |
| Formulation A | SD | 397 | 349 | 540 | 479 | 8251 | 921 | 110 | 753 |
| (n = 12) | % RSD | 11 | 5.6 | 31 | 23 | 157.7 | 78 | 21 | 81 |
| Group 2 Male | Average | 4433 | 6173 | 5309 | 2957 | 2087 | 1332 | 564 | 879 |
| Formulation B | SD | 2581 | 2665 | 7076 | 1562 | 2098 | 774 | 121 | 580 |
| (n = 12) | % RSD | 58 | 43 | 133 | 53 | 101 | 58 | 21 | 66 |

Lens (ng/g)

| | | | | | | | | | |
|---|---|---|---|---|---|---|---|---|---|
| Group 1 Male | Average | 74.6 | 59.2 | 20.5 | 23.9 | 13.3 | 28.5 | 19.3 | 12.2 |
| Formulation A | SD | 71 | 7.1 | 2.1 | 11 | 3.4 | 32 | 17 | 4.2 |
| (n = 12) | % RSD | 95 | 12 | 10 | 46 | 26 | 112 | 89 | 35 |
| Group 2 Male | Average | 48.0 | 56.7 | 12.6 | 19.9 | 11.0 | 26.5 | 10.5 | 21.7 |
| Formulation B | SD | 12 | 24 | 4.0 | 3.3 | 3.5 | 30 | 2.5 | 6.1 |
| (n = 12) | % RSD | 25 | 42 | 31 | 17 | 32 | 112 | 23 | 28 |

Retina-Choroid (ng/g)

| | | | | | | | | | |
|---|---|---|---|---|---|---|---|---|---|
| Group 1 Male | Average | 4120 | 2955 | 2621 | 1735 | 2660 | 1023 | 848 | 1086 |
| Formulation A | SD | 2024 | 777 | 2754 | 662 | 3239 | 1186 | 339 | 993 |
| (n = 12) | % RSD | 49 | 26 | 105 | 38 | 122 | 116 | 40 | 91 |
| Group 2 Male | Average | 2740 | 6136 | 2520 | 2800 | 744 | 1610 | 568 | 933 |
| Formulation B | SD | 835 | 6225 | 3568 | 2559 | 878 | 1839 | 624 | 388 |
| (n = 12) | % RSD | 30 | 101 | 142 | 91 | 118 | 114 | 110 | 42 |

Sclera (ng/g)

| | | | | | | | | | |
|---|---|---|---|---|---|---|---|---|---|
| Group 1 Male | Average | 6111 | 7840 | 8661 | 4045 | 15067 | 7365 | 1325 | 1005 |
| Formulation A | SD | 3708 | 2874 | 11531 | 3876 | 24018 | 11877 | 832 | 342 |
| (n = 12) | % RSD | 61 | 37 | 133 | 96 | 159 | 161 | 63 | 34 |
| Group 2 Male | Average | 7153 | 11680 | 1237 | 7499 | 1352 | 2592 | 1131 | 2560 |
| Formulation B | SD | 5380 | 7102 | 1027 | 8346 | 1487 | 3021 | 1187 | 1385 |
| (n = 12) | % RSD | 75 | 61 | 83 | 111 | 110 | 117 | 105 | 54 |

Conjunctivas (ng/g)

| | | | | | | | | | |
|---|---|---|---|---|---|---|---|---|---|
| Group 1 Male | Average | 8423 | 16213 | 1296 | 15189 | 2860 | 10917 | 7407 | 6427 |
| Formulation A | SD | 8005 | 14314 | 312 | 12527 | 2201 | 16855 | 6940 | 650 |
| (n = 12) | % RSD | 95 | 88 | 24 | 82 | 77 | 154 | 94 | 10 |
| Group 2 Male | Average | 1931 | 84427 | 1857 | 10520 | 1346 | 26253 | 1381 | 17824 |
| Formulation B | SD | 1189 | 68982 | 1432 | 183 | 942 | 33392 | 1218 | 22700 |
| (n = 12) | % RSD | 62 | 82 | 77 | 1.7 | 70 | 127 | 88 | 127 |

Cornea (ng/g)

| | | | | | | | | | |
|---|---|---|---|---|---|---|---|---|---|
| Group 1 Male | Average | 14760 | 13467 | 4556 | 5597 | 3909 | 3272 | 1020 | 2973 |
| Formulation A | SD | 3916 | 2171 | 3021 | 2809 | 5864 | 4787 | 673 | 1739 |
| (n = 12) | % RSD | 27 | 16 | 66 | 50 | 150 | 146 | 66 | 58 |
| Group 2 Male | Average | 18187 | 32653 | 2699 | 5965 | 1028 | 3381 | 303 | 1834 |
| Formulation B | SD | 6082 | 15119 | 1113 | 2513 | 698 | 2641 | 72 | 2.8 |
| (n = 12) | % RSD | 33 | 46 | 41 | 42 | 68 | 78 | 24 | 0.15 |

Rapid exposure to Compound 1 was evident in all animals administered with Formulation A or Formulation B as indicated by plasma and ocular tissue maximum exposure values (Cₘₐₓ) at the first sampling, one hour post-dose. The terminal plasma half-life (T_{1/2,e}) was 3 hr for both formulations with systemic clearances (Cl/F) of 8.7 L/hr/kg for Formulation A and 6.4 L/hr/kg for Formulation B. The apparent volume of distribution (V/F) was 374 L/kg for Formulation A and 287 L/kg for Formulation B.

The rank order of Cₘₐₓ (after 1 hour in most cases) from each formulation and in each dosed eye was as follows:
Formulation A Right Eye—Conjunctivas≈Cornea>>Sclera>Iris/Ciliary Body>Retina-Choroid-Plexus>>Aqueous Humor>>Vitreous Humor>Lens;
Formulation A Left Eye—Sclera≈Cornea>>Conjunctivas>Iris/Ciliary Body>Retina-Choroid-Plexus>Aqueous Humor>Vitreous Humor>>Lens;
Formulation B Right Eye—Conjunctivas>>Cornea>>Sclera>Iris/Ciliary Body>Retina-Choroid-Plexus>Aqueous Humor>Vitreous Humor>>Lens;
Formulation B Left Eye—Cornea>>Sclera>Iris/Ciliary Body>Retina-Choroid-Plexus>Conjunctivas≈Aqueous Humor>>Vitreous Humor>>Lens.

The rank order of the total exposure (AUC_{0-last exposure}) (0-12 hours) from each formulation and dosed eye was as follows:
Formulation A Right Eye—Conjunctivas>Cornea≈Sclera>Iris/Ciliary Body>Retina-Choroid-Plexus>>Aqueous Humor>Vitreous Humor>Lens;
Formulation A Left Eye—Sclera>Cornea>Conjunctivas>Iris/Ciliary Body>Retina-Choroid-Plexus>>Aqueous Humor>Vitreous Humor>Lens;
Formulation B Right Eye—Conjunctivas>Cornea>Sclera>Retina-Choroid-Plexus Iris/Ciliary Body>Aqueous Humor>Vitreous Humor>Lens;
Formulation B Left Eye—Cornea>Iris/Ciliary Body>Sclera>Conjunctivas≈Retina-Choroid-Plexus>Aqueous Humor>Vitreous Humor>Lens.

No substantial differences were found between Formulation A and Formulation B in the systemic and ocular tissue exposures to Compound 1 in male Dutch-belted rabbits after bilateral topical ocular administration at a dose of 2.5 mg/eye.

### Example 14—Container Closure System and Dose Delivery System

A low-density translucent polyethylene dropper bottle (5 mL with a 0.05 mL nozzle) and matching cap are used as a container closure system and dose delivery device. The dropper is designed to deliver a 50 μL drop, which is appropriate for ophthalmic delivery. BAC (0.02% w/v) is added to the formulation as an antimicrobial reagent to support use of the container system for multi-dose delivery.

Every 14 days, each study subject receives a fresh supply of ophthalmic formulations. Each study subject is given a labeled box containing three dropper bottles at the first study visit along with instructions to use one bottle for the first treatment week, a second bottle for the second treatment week, and a third bottle as a spare. Upon the subject's return for the second study visit after 14 days, all three bottles are collected. The subject is provided with another labeled box of three dropper bottles, with instructions to use one bottle for the third treatment week, a second bottle for the fourth treatment week, and a third bottle as a spare.

The labeled dropper bottles may be packaged into a secondary packaging, such as cardboard cartons, which can be further packaged.

ICH compliant stability studies may be performed to assure that the ophthalmic formulations comply with the release specifications for the duration of the prescribed study period (including accelerated temperature conditions and low humidity storage to confirm that shipping and post-dispensing temperature deviations will not adversely affect product potency and purity, and that excessive evaporation of the formulation will not occur).

### Example 15—Antimicrobial Effectiveness Testing

Antimicrobial Effectiveness Testing (AET) in accordance with USP <51> may be conducted for Compound 1 ophthalmic formulations. The USP-specified microbial species and strains include Staphylococcus aureus ATCC 6538, Pseudomonas aeruginosa ATCC 9027, Clostridium sporogenes ATCC 11437, Bacillus subtilis ATCC 6633, Candida albicans ATCC 10231, and Aspergillus brasiliensis ATCC 16404. To support antimicrobial testing, an ophthalmic formulation of Compound 1 is filled into polypropylene multi-dose eye dropper bottle (where the drug product is compounded in, at minimum, a Class 10,000 certified work area, and undergoes sterile filtration using a 0.45 μm bioburden reductive filtration followed by redundant 0.22 μm filtrations; whereafter the drug product is then filled into a pre-sterilized dropper bottle).

For release testing in accordance with USP <71> standards, the product is tested for sterility by a method that is capable of detecting a 100 colony-forming units (CFU) spike level of USP <71> recommended microorganisms. Effectiveness in preventing growth of a microbial challenge is demonstrated as follows:
Bacterial Growth Suppression Effectiveness Requirements
   No less than a 1-log (90%) reduction from the initial calculated count after 7 days, No less than a 3-log (99.9%) reduction from the initial calculated count after 14 days,
   No increase from the initial calculated count between 14 and 28 day;
Yeast and Molds Suppression Effectiveness Requirements
   No increase from the initial count at 7, 14 or 28 days,
   No increase from the initial calculated count at 7, 14 and 28 days.

### Example 16—Storage Stability Analysis of a Compound 1 Ophthalmic Formulation

Aqueous Formulation A from Example 1 above was subjected to stability analysis by storage at the conditions specified below (i.e., 5° C., 25° C. at 40% relative humidity, or 30° C. at 65% relative humidity). The physical appearance of the formulation was evaluated throughout the study, and aliquots of the formulation were analyzed by liquid chromatography using a UV-Vis detector to determine the amount of Compound 1 in the formulation, along with the amount of Related Substances in the formulation. Aliquots of formulation were also tested to determine the pH of the solution and the amount of Particulate Matter. Results are provided in the tables below, which provide (i) results at each of 0, 1, 6, 9, 12, and 18 months for samples of Aqueous Formulation A stored at 5° C. or stored at 25° C. at 40% relative humidity, and (ii) results at each of 0, 1, and 6 months for samples of Aqueous Formulation A stored at 30° C. at 65% relative humidity. The abbreviation RRT refers to relative retention time in the liquid chromatography chromatogram. The results show that Aqueous Formulation A has good stability upon storage at the conditions tested.

TABLE 1
Stability Study Results from Experiment at 5° C.

| | Duration of Storage of Formulation | | | |
|---|---|---|---|---|
| Test Parameter | Initial (0 Months) | 1 Month | 6 Months | 9 Months |
| Area Percent of | 96.5% | 96.7% | 95.2% | 94.8% |
| Compound 1 in | | | | |
| Liquid | | | | |
| Chromatography | | | | |
| Chromatogram | | | | |
| Area Percent of | RRT 0.92: 0.06% | RRT 0.92: 0.06% | RRT 0.92: 0.06% | RRT 0.92: 0.06% |
| Related | RRT 0.96: 0.32% | RRT 0.96: 0.32% | RRT 0.96: 0.31% | RRT 0.96: 0.31% |
| Substances in | RRT 1.04: 0.06% | RRT 1.04: 0.07% | RRT 1.04: 0.06% | RRT 1.04: 0.06% |
| Liquid | RRT 1.106: 0.14% | RRT 1.09: 0.05% | RRT 1.09: 0.05% | RRT 1.09: <0.05% |
| Chromatography | RRT 1.11: 0.09% | RRT 1.106: 0.14% | RRT 1.106: 0.14% | RRT 1.106: 0.13% |
| Chromatogram | RRT 1.12: 0.10% | RRT 1.11: 0.12% | RRT 1.11: 0.12% | RRT 1.11: 0.09% |
| | RRT 1.14: 0.05% | RRT 1.12: 0.13% | RRT 1.12: 0.13% | RRT 1.12: 0.07% |
| | RRT 1.29: 0.06% | RRT 1.14: 0.08% | RRT 1.14: 0.09% | RRT 1.14: <0.05% |
| | Total Related | RRT 1.29: 0.07% | RRT 1.29: 0.09% | RRT 1.29: 0.06% |
| | Substances: 0.88% | Total Related | Total Related | Total Related |
| | | Substances: 1.0% | Substances: 1.0% | Substances: 0.77% |
| Formulation | Colorless solution | Clear, light brown | Clear, very slightly | Clear, light brown |
| Appearance | | solution | brown solution | solution |
| pH of | 8.0 | 8.0 | 8.0 | 8.0 |
| Formulation | | | | |
| Particulate | Particles ≥ 10 μm: | Particles ≥ 10 μm: | Particles ≥ 10 μm: | Particles ≥ 10 μm: 0 |
| Matter in | 2 particles/mL | 3 particles/mL | 2 particles/mL | particles/mL |
| Formulation | Particles ≥ 25 μm: | Particles ≥ 25 μm: | Particles ≥ 25 μm: | Particles ≥ 25 μm: 0 |
| | 0 particles/mL | 0 particles/mL | 1 particle/mL | particles/mL |
| | Particles ≥ 50 μm: | Particles ≥ 50 μm: | Particles ≥ 50 μm: | Particles ≥ 50 μm: 0 |
| | 0 particles/mL | 0 particles/mL | 0 particles/mL | particles/mL |

Duration of Storage of Formulation

| Test Parameter | 12 Months | 18 Months |
|---|---|---|
| Area Percent of Compound 1 in | 95.8% | 96.5% |
| Liquid Chromatography | | |
| Chromatogram | | |
| Area Percent of Related | RRT 0.92: 0.06% | RRT 0.92: 0.06% |
| Substances in Liquid | RRT 0.96: 0.31% | RRT 0.96: 0.32% |
| Chromatography | RRT 1.04: 0.06% | RRT 1.04: 0.06% |
| Chromatogram | RRT 1.09: <0.05% | RRT 1.09: <0.05% |
| | RRT 1.106: 0.13% | RRT 1.106: 0.15% |
| | RRT 1.11: 0.09% | RRT 1.11: 0.09% |
| | RRT 1.12: 0.08% | RRT 1.12: 0.08% |
| | RRT 1.14: <0.05% | RRT 1.29: 0.07% |
| | RRT 1.29: 0.06% | Total Related Substances: 0.83% |
| | Total Related Substances: 0.79% | |
| Formulation Appearance | Clear, light brown | Clear, light brown |
| | solution | solution |
| pH of Formulation | 8.0 | 8.0 |
| Particulate | Particles ≥ 10 μm: 3 particles/mL | Particles ≥ 10 μm: 1 particle/mL |
| Matter in | Particles ≥ 25 μm: 1 particle/mL | Particles ≥ 25 μm: 0 particles/mL |
| Formulation | Particles ≥ 50 μm: 0 particles/mL | Particles ≥ 50 μm: 0 particles/mL |

TABLE 2
Stability Study Results from Experiment at 25° C. at 40% Relative Humidity.

| | Duration of Storage of Formulation | | | |
|---|---|---|---|---|
| Test Parameter | Initial (0 Months) | 1 Month | 6 Months | 9 Months |
| Area Percent of | 96.5% | 96.4% | 95.4% | 95.2% |
| Compound 1 in | | | | |
| Liquid | | | | |
| Chromatography | | | | |
| Chromatogram | | | | |
| Area Percent of | RRT 0.92: 0.06% | RRT 0.92: 0.06% | RRT 0.92: 0.06% | RRT 0.92: 0.06% |
| Related | RRT 0.96: 0.32% | RRT 0.96: 0.32% | RRT 0.96: 0.33% | RRT 0.96: 0.33% |
| Substances in | RRT 1.04: 0.06% | RRT 1.04: 0.07% | RRT 1.04: 0.06% | RRT 1.04: 0.06% |
| Liquid | RRT 1.106: 0.14% | RRT 1.083: 0.05% | RRT 1.083: 0.11% | RRT 1.076: 0.11% |
| Chromatography | RRT 1.11: 0.09% | RRT 1.09: 0.05% | RRT 1.09: <0.05% | RRT 1.083: not |
| Chromatogram | RRT 1.12: 0.10% | RRT 1.106: 0.15% | RRT 1.106: 0.14% | detected |
| | RRT 1.14: 0.05% | RRT 1.11: 0.11% | RRT 1.11: 0.12% | RRT 1.09: <0.05% |
| | RRT 1.29: 0.06% | RRT 1.12: 0.12% | RRT 1.12: 0.12% | RRT 1.106: 0.12% |
| | Total Related | RRT 1.14: 0.07% | RRT 1.14: 0.07% | RRT 1.11: 0.10% |
| | Substances: | RRT 1.29: 0.07% | RRT 1.29: 0.09% | RRT 1.12: 0.07% |
| | 0.88% | Total Related | Total Related | RRT 1.14: <0.05% |
| | | Substances: 1.1% | Substances: 1.1% | RRT 1.29: 0.06% |
| | | | | Total Related |
| | | | | Substances: |
| | | | | 0.92% |
| Formulation | Colorless solution | Clear, light brown | Clear, very slightly | Clear, light brown |
| Appearance | | solution | brown | solution |
| | | | solution | |
| pH of | 8.0 | 8.0 | 8.0 | 8.0 |
| Formulation | | | | |
| Particulate | Particles ≥ 10 μm: | Particles ≥ 10 μm: | Particles ≥ 10 μm: | Particles ≥ 10 μm: |
| Matter in | 2 particles/mL | 2 particles/mL | 1 particle/mL | 1 particle/mL |
| Formulation | Particles ≥ 25 μm: | Particles ≥ 25 μm: | Particles ≥ 25 μm: | Particles ≥ 25 μm: |
| | 0 particles/mL | 1 particle/mL | 0 particles/mL | 0 particles/mL |
| | Particles ≥ 50 μm: | Particles ≥ 50 μm: | Particles ≥ 50 μm: | Particles ≥ 50 μm: |
| | 0 particles/mL | 0 particles/mL | 0 particles/mL | 0 particles/mL |

| | Duration of Storage of Formulation | |
|---|---|---|
| Test Parameter | 12 Months | 18 Months |
| Area Percent of Compound 1 in | 97.0% | 98.1% |
| Liquid Chromatography | | |
| Chromatogram | | |
| Area Percent of Related | RRT 0.92: 0.06% | RRT 0.91: 0.06% |
| Substances in Liquid | RRT 0.96: 0.34% | RRT 0.92: 0.06% |
| Chromatography | RRT 1.04: 0.05% | RRT 0.96: 0.35% |
| Chromatogram | RRT 1.076: 0.11% | RRT 1.04: 0.05% |
| | RRT 1.083: not detected | RRT 1.076: 0.12% |
| | RRT 1.09: <0.05% | RRT 1.083: not detected |
| | RRT 1.106: 0.12% | RRT 1.09: <0.05% |
| | RRT 1.11: 0.12% | RRT 1.106: 0.13% |
| | RRT 1.12: 0.08% | RRT 1.11: 0.14% |
| | RRT 1.14: <0.05% | RRT 1.12: 0.08% |
| | RRT 1.24: 0.06% | RRT 1.14: <0.05% |
| | RRT 1.29: 0.06% | RRT 1.24: 0.07% |
| | Total Related | RRT 1.29: 0.07% |
| | Substances: 1.0% | Total Related Substances: 1.1% |
| Formulation Appearance | Clear, light brown | Clear, light brown |
| | solution | solution |
| pH of Formulation | 8.0 | 8.0 |
| Particulate | Particles ≥ 10 μm: | Particles ≥ 10 μm: |
| Matter in | 3 particles/mL | 1 particle/mL |
| Formulation | Particles ≥ 25 μm: | Particles ≥ 25 μm: |
| | 1 particle/mL | 0 particles/mL |
| | Particles ≥ 50 μm: | Particles ≥ 50 μm: |
| | 0 particles/mL | 0 particles/mL |

TABLE 3
Stability Study Results from Experiment at 30° C. at 65% Relative Humidity

| | Duration of Storage of Formulation | | |
|---|---|---|---|
| Test Parameter | Initial (0 Months) | 1 Month | 6 Months |
| Area Percent of | 96.5% | 96.7% | 95.4% |
| Compound 1 in Liquid | | | |
| Chromatography | | | |
| Chromatogram | | | |
| Area Percent of Related | RRT 0.92: 0.06% | RRT 0.92: 0.06% | RRT 0.92: 0.06% |
| Substances in Liquid | RRT 0.96: 0.32% | RRT 0.96: 0.33% | RRT 0.96: 0.33% |
| Chromatography | RRT 1.04: 0.06% | RRT 1.04: 0.06% | RRT 1.04: 0.05% |
| Chromatogram | RRT 1.106: 0.14% | RRT 1.083: 0.07% | RRT 1.083: 0.12% |
| | RRT 1.11: 0.09% | RRT 1.09: 0.05% | RRT 1.09: <0.05% |
| | RRT 1.12: 0.10% | RRT 1.106: 0.14% | RRT 1.12: 0.12% |
| | RRT 1.14: 0.05% | RRT 1.11: 0.11% | RRT 1.14: 0.07% |
| | RRT 1.29: 0.06% | RRT 1.12: 0.12% | RRT 1.24: 0.06% |
| | Total Related | RRT 1.14: 0.07% | RRT 1.29: 0.09% |
| | Substances: 0.88% | RRT 1.29: 0.07% | Total Related Substances: |
| | | Total Related Substances: | 1.2% |
| | | 1.1% | |
| Formulation Appearance | Colorless solution | Clear, light brown | Clear, very slightly brown |
| | | solution | solution |
| pH of Formulation | 8.0 | 8.0 | 8.0 |
| Particulate | Particles ≥ 10 μm: 2 | Particles ≥ 10 μm: 1 | Particles ≥ 10 μm: 1 |
| Matter in | particles/mL | particle/mL | particle/mL |
| Formulation | Particles ≥ 25 μm: 0 | Particles ≥ 25 μm: 0 | Particles ≥ 25 μm: 0 |
| | particles/mL | particles/mL | particles/mL |
| | Particles ≥ 50 μm: 0 | Particles ≥ 50 μm: 0 | Particles ≥ 50 μm: 0 |
| | particles/mL | particles/mL | particles/mL |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. An aqueous, ophthalmic solution, comprising:
a. from 4.7% (w/v) to 5.3% (w/v) of a compound of Formula I: 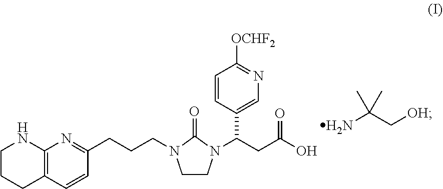
b. from 14% (w/v) to 18% (w/v) of a cyclodextrin;
c. from 0.1% (w/v) to 5% (w/v) of a buffer;
d. from 0.01% (w/v) to 1% (w/v) of a preservative; and
e. at least 75% (w/v) water;
wherein the solution has a pH in the range of 7.5 to 8.7.

2. The solution of claim 1, wherein the cyclodextrin is 2-hydroxypropyl-β-cyclodextrin.

3. The solution of claim 1 or 2, wherein the solution comprises from 15% (w/v) to 18% (w/v) of the cyclodextrin.

4. The solution of claim 1 or 2, wherein the solution comprises from 15% (w/v) to 17% (w/v) of the cyclodextrin.

5. The solution of claim 1 or 2, wherein the solution comprises from 15% (w/v) to 16% (w/v) of the cyclodextrin.

6. The solution of claim 1 or 2, wherein the solution comprises from 15.2% (w/v) to 15.8% (w/v) of the cyclodextrin.

7. The solution of claim 1 or 2, wherein the solution comprises from 15.3% (w/v) to 15.7% (w/v) of the cyclodextrin.

8. The solution of claim 1 or 2, wherein the solution comprises from 15.4% (w/v) to 15.6% (w/v) of the cyclodextrin.

9. The solution of claim 1 or 2, wherein the solution comprises 15.5% (w/v) of the cyclodextrin.

10. The solution of any one of claims 1-9, wherein the buffer comprises an organic acid

11. The solution of any one of claims 1-9, wherein the buffer comprises boric acid.

12. The solution of any one of claims 1-9, wherein the buffer is a mixture of boric acid and an alkali metal borate.

13. The solution of any one of claims 1-9, wherein the buffer is a mixture of boric acid and sodium borate.

14. The solution of any one of claims 1-13, wherein the solution comprises from 0.1% (w/v) to 2.5% (w/v) of the buffer.

15. The solution of any one of claims 1-13, wherein the solution comprises from 0.25% (w/v) to 2.5% (w/v) of the buffer.

16. The solution of any one of claims 1-13, wherein the solution comprises from 0.5% (w/v) to 1.5% (w/v) of the buffer.

17. The solution of any one of claims 1-13, wherein the solution comprises from 0.75% (w/v) to 1.25% (w/v) of the buffer.

18. The solution of any one of claims 1-13, wherein the solution comprises from 0.8% (w/v) to 1.2% (w/v) of the buffer.

19. The solution of any one of claims 1-13, wherein the solution comprises from 0.9% (w/v) to 1.1% (w/v) of the buffer.

20. The solution of any one of claims 1-13, wherein the solution comprises 1% (w/v) of the buffer.

21. The solution of any one of claims 1-20, wherein the preservative comprises a benzalkonium salt.

22. The solution of any one of claims 1-20, wherein the preservative comprises a benzalkonium halide.

23. The solution of any one of claims 1-20, wherein the preservative comprises benzalkonium chloride.

24. The solution of any one of claims 1-23, wherein the solution comprises from 0.01% (w/v) to 0.1% (w/v) of the preservative.

25. The solution of any one of claims 1-23, wherein the solution comprises from 0.01% (w/v) to 0.05% (w/v) of the preservative.

26. The solution of any one of claims 1-23, wherein the solution comprises from 0.012% (w/v) to 0.05% (w/v) of the preservative.

27. The solution of any one of claims 1-23, wherein the solution comprises from 0.015% (w/v) to 0.05% (w/v) of the preservative.

28. The solution of any one of claims 1-23, wherein the solution comprises from 0.012% (w/v) to 0.03% (w/v) of the preservative.

29. The solution of any one of claims 1-23, wherein the solution comprises from 0.012% (w/v) to 0.025% (w/v) of the preservative.

30. The solution of any one of claims 1-23, wherein the solution comprises from 0.015% (w/v) to 0.025% (w/v) of the preservative.

31. The solution of any one of claims 1-23, wherein the solution comprises from 0.012% (w/v) to 0.02% (w/v) of the preservative.

32. The solution of any one of claims 1-23, wherein the solution comprises from 0.015% (w/v) to 0.02% (w/v) of the preservative.

33. The solution of any one of claims 1-23, wherein the solution comprises 0.02% (w/v) of the preservative.

34. The solution of any one of claims 1-33, further comprising a chelating agent.

35. The solution of any one of claims 1-33, further comprising from 0.001% (w/v) to 2% (w/v) of a chelating agent.

36. The solution of any one of claims 1-33, further comprising from 0.01% (w/v) to 1% (w/v) of a chelating agent.

37. The solution of any one of claims 1-33, further comprising from 0.01% (w/v) to 0.5% (w/v) of a chelating agent.

38. The solution of any one of claims 1-33, further comprising from 0.05% (w/v) to 0.5% (w/v) of a chelating agent.

39. The solution of any one of claims 1-33, further comprising from 0.05% (w/v) to 0.1% (w/v) of a chelating agent.

40. The solution of any one of claims 1-33, further comprising from 0.01% (w/v) to 0.1% (w/v) of a chelating agent.

41. The solution of any one of claims 1-33, further comprising 0.1% (w/v) of a chelating agent.

42. The solution of any one of claims 34-41, wherein the chelating agent comprises ethylenediaminetetraacetic acid or a salt thereof.

43. The solution of any one of claims 34-41, wherein the chelating agent is sodium ethylenediaminetetraacetate.

44. The solution of any one of claims 1-43, wherein the solution comprises at least 77% (w/v) water.

45. The solution of any one of claims 1-43, wherein the solution comprises at least 78% (w/v) water.

46. The solution of any one of claims 1-45, wherein the solution has a pH in the range of 7.5 to 8.5.

47. The solution of any one of claims 1-45, wherein the solution has a pH in the range of 7.8 to 8.5.

48. The solution of any one of claims 1-45, wherein the solution has a pH in the range of 7.8 to 8.2.

49. The solution of any one of claims 1-45, wherein the solution has a pH in the range of 7.9 to 8.1.

50. The solution of any one of claims 1-45, wherein the solution has a pH of 8.0.

51. The solution of any one of claims 1-50, wherein the cyclodextrin has a molecular weight in the range of from about 1200 g/mol to about 1600 g/mol.

52. The solution of any one of claims 1-50, wherein the cyclodextrin has a molecular weight in the range of from about 1300 g/mol to about 1500 g/mol.

53. The solution of any one of claims 1-50, wherein the cyclodextrin has a molecular weight in the range of from about 1350 g/mol to about 1450 g/mol.

54. The solution of any one of claims 1-50, wherein the cyclodextrin has a molecular weight of about 1400 g/mol.

55. The solution of any one of claims 1-54, wherein the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.85.

56. The solution of any one of claims 1-54, wherein the cyclodextrin is a 2-hydroxypropyl-β-cyclodextrin in which the mole ratio of 2-hydroxylpropyl substituents per glucose moiety in the β-cyclodextrin is in the range of from about 0.5 to about 0.8.

57. The solution of any one of claims 1-54, wherein the cyclodextrin is a 2-hydroxypropyl-β-cyclodextrin in which the mole ratio of 2-hydroxylpropyl substituents per glucose moiety in the β-cyclodextrin is in the range of from about 0.55 to about 0.77.

58. The solution of any one of claims 1-54, wherein the cyclodextrin is a 2-hydroxypropyl-β-cyclodextrin in which the mole ratio of 2-hydroxylpropyl substituents per glucose moiety in the β-cyclodextrin is in the range of from about 0.59 to about 0.73.

59. The solution of any one of claims 1-58, further comprising a tonicity modifier.

60. The solution of any one of claims 1-58, further comprising about 0.01% (w/w) to about 5% (w/w) of a tonicity modifier.

61. The solution of any one of claims 1-58, further comprising about 0.1% (w/w) to about 2% (w/w) of a tonicity modifier.

62. An aqueous, ophthalmic solution, comprising:
a. from 4.9% (w/v) to 5.1% (w/v) of a compound of Formula I: 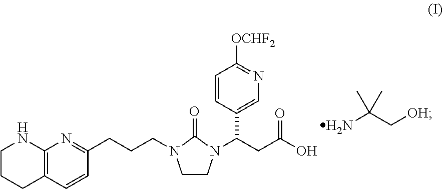
b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.2% (w/v) of a benzalkonium salt; and
e. at least 75% (w/v) water;
wherein the solution has a pH in the range of 7.8 to 8.5.

63. An aqueous, ophthalmic solution, comprising:
a. 5.5% (w/v) of a compound of Formula I: 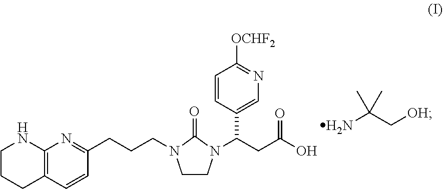
b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water;
wherein the solution has a pH in the range of 7.8 to 8.5.

64. An aqueous, ophthalmic solution, comprising:
a. 5.5% (w/v) of a compound of Formula I: 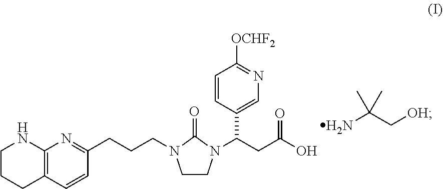
b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water;
wherein the solution has a pH in the range of 7.8 to 8.5.

65. The solution of any one of claims 1-65, wherein the solution contains less than 1% (w/w) of a precipitate that is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid hydrate.

66. The solution of any one of claims 1-65, wherein the solution contains less than 0.5% (w/w) of a precipitate that is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid hydrate.

67. The solution of any one of claims 1-65, wherein the solution contains less than 0.1% (w/w) of a precipitate that is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid hydrate.

68. An aqueous, ophthalmic solution, consisting of:
a. from 4.9% (w/v) to 5.1% (w/v) of a compound of Formula I: 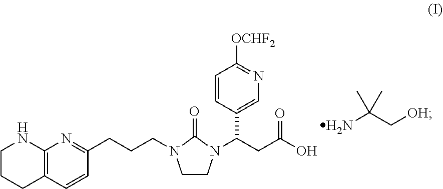
b. from 15% (w/v) to 16% (w/v) of 2-hydroxypropyl-β-cyclodextrin;
c. from 0.5% (w/v) to 1.5% (w/v) of a buffer comprising boric acid;
d. from 0.01% (w/v) to 0.5% (w/v) of a benzalkonium salt;
e. at least 75% (w/v) water; and
f. one or more excipients independently selected from the group consisting of a pH adjuster and a tonicity modifier;
wherein the solution has a pH in the range of 7.8 to 8.5.

69. An aqueous, ophthalmic solution, consisting of:
a. 5.5% (w/v) of a compound of Formula I: 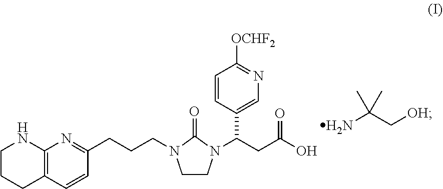
b. about 15.5% (w/v) of 2-hydroxypropyl-β-cyclodextrin;
c. about 1% (w/v) of a buffer comprising boric acid;
d. about 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

70. An aqueous, ophthalmic solution, consisting of:
a. 5.5% (w/v) of a compound of Formula I: 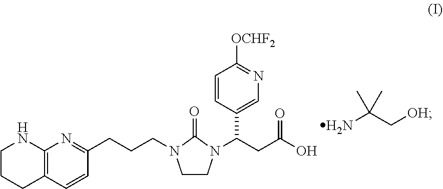
b. 15.5% (w/v) of 2-hydroxypropyl-β-cyclodextrin;
c. 1% (w/v) of a buffer comprising boric acid;
d. 0.02% (w/v) of a benzalkonium salt; and
e. at least 75% (w/v) water; and
f. one or more excipients independently selected from the group consisting of a pH adjuster and a tonicity modifier;
wherein the solution has a pH in the range of 7.8 to 8.5.

71. The solution of any one of claims 68-70, wherein the one or more excipients is a pH adjuster.

72. The solution of any one of claims 62-71, wherein the benzalkonium salt is a benzalkonium halide.

73. The solution of any one of claims 62-71, wherein the benzalkonium salt is benzalkonium chloride.

74. The solution of any one of claims 62-73, wherein the 2-hydroxypropyl-β-cyclodextrin has a molecular weight in the range of from about 1300 g/mol to about 1500 g/mol.

75. The solution of any one of claims 62-73, wherein the 2-hydroxypropyl-β-cyclodextrin has a molecular weight in the range of from about 1350 g/mol to about 1450 g/mol.

76. The solution of any one of claims 62-73, wherein the 2-hydroxypropyl-β-cyclodextrin has a molecular weight of about 1400 g/mol.

77. The solution of any one of claims 62-76, wherein the mole ratio of 2-hydroxylpropyl substituents per glucose moiety in the 2-hydroxypropyl-β-cyclodextrin is in the range of from about 0.5 to about 0.85.

78. The solution of any one of claims 62-76, wherein the mole ratio of 2-hydroxylpropyl substituents per glucose moiety in the 2-hydroxypropyl-β-cyclodextrin is in the range of from about 0.5 to about 0.8.

79. The solution of any one of claims 62-76, wherein the mole ratio of 2-hydroxylpropyl substituents per glucose moiety in the 2-hydroxypropyl-β-cyclodextrin is in the range of from about 0.55 to about 0.77.

80. The solution of any one of claims 62-76, wherein the mole ratio of 2-hydroxylpropyl substituents per glucose moiety in the 2-hydroxypropyl-β-cyclodextrin is in the range of from about 0.59 to about 0.73.

81. The solution of any one of claims 62-80, wherein the solution has a pH in the range of 7.8 to 8.2.

82. The solution of any one of claims 62-80, wherein the solution has a pH of 8.0.

83. The solution of any one of claims 1-82, wherein less than 1% of the compound of Formula I degrades upon storage at 25° C. for 2 weeks.

84. The solution of any one of claims 1-82, wherein less than 0.5% of the compound of Formula I degrades upon storage at 25° C. for 2 weeks.

85. The solution of any one of claims 1-82, wherein less than 0.1% of the compound of Formula I degrades upon storage at 25° C. for 2 weeks.

86. The solution of any one of claims 1-85, wherein less than 1% of the compound of Formula I degrades upon storage at 25° C. for 24 weeks.

87. The solution of any one of claims 1-85, wherein less than 0.5% of the compound of Formula I degrades upon storage at 25° C. for 24 weeks.

88. The solution of any one of claims 1-87, wherein less than 1% of the compound of Formula I degrades upon storage at 40° C. for 2 weeks.

89. The solution of any one of claims 1-87, wherein less than 0.5% of the compound of Formula I degrades upon storage at 40° C. for 2 weeks.

90. The solution of any one of claims 1-87, wherein less than 0.1% of the compound of Formula I degrades upon storage at 40° C. for 2 weeks.

91. The solution of any one of claims 1-90, wherein less than 1% of the compound of Formula I degrades upon storage at 40° C. for 24 weeks.

92. The solution of any one of claims 1-90, wherein less than 0.5% of the compound of Formula I degrades upon storage at 40° C. for 24 weeks.

93. The solution of any one of claims 1-92, wherein storage of the solution at 25° C. for 2 weeks results in a formulation containing less than 1% (w/w) of any solid precipitate that forms from the solution.

94. The solution of any one of claims 1-92, wherein storage of the solution at 25° C. for 24 weeks results in a formulation containing less than 1% (w/w) of any solid precipitate that forms from the solution.

95. The solution of any one of claims 1-94, wherein storage of the solution at 40° C. for 2 weeks results in a formulation containing less than 1% (w/w) of any solid precipitate that forms from the solution.

96. The solution of any one of claims 1-95, wherein storage of the solution at 40° C. for 24 weeks results in a formulation containing less than 1% (w/w) of any solid precipitate that forms from the solution.

97. The solution of any one of claims 1-92, wherein after storage of the solution at 25° C. for 2 weeks, there is no solid precipitate that forms from the solution.

98. The solution of any one of claims 1-92, wherein after storage of the solution at 25° C. for 24 weeks, there is no solid precipitate that forms from the solution.

99. The solution of any one of claims 1-92, wherein after storage of the solution at 40° C. for 2 weeks, there is no solid precipitate that forms from the solution.

100. The solution of any one of claims 1-92, wherein after storage of the solution at 40° C. for 24 weeks, there is no solid precipitate that forms from the solution.

101. The solution of any one of claims 93-100, wherein the solid precipitate comprises (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

102. The solution of any one of claims 93-100, wherein the solid precipitate is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

103. An aqueous, ophthalmic solution, comprising:
a. from about 4% (w/v) to about 6% (w/v) of a compound of Formula II or a pharmaceutically acceptable salt thereof: 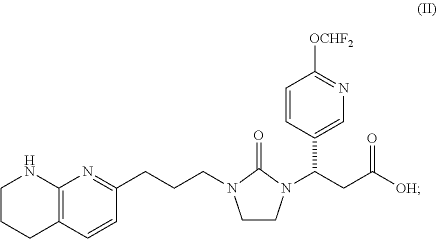 [SMILES: O=C(O)C[C@@H](c1ccc(OC(F)F)nc1)N1CCN(CCCc2ccc3c(n2)NCCC3)C1=O]
b. from 14% (w/v) to 18% (w/v) of a cyclodextrin;
c. a buffer;
d. a preservative; and
e. at least 75% (w/v) water;
wherein the solution has a pH in the range of 7.5 to 8.7.

104. The solution of claim 103, wherein the cyclodextrin is 2-hydroxypropyl-β-cyclodextrin.

105. The solution of claim 103 or 104, wherein the solution comprises from 15% (w/v) to 18% (w/v) of the cyclodextrin.

106. The solution of claim 103 or 104, wherein the solution comprises from 15% (w/v) to 17% (w/v) of the cyclodextrin.

107. The solution of claim 103 or 104, wherein the solution comprises from 15% (w/v) to 16% (w/v) of the cyclodextrin.

108. The solution of claim 103 or 104, wherein the solution comprises from 15.2% (w/v) to 15.8% (w/v) of the cyclodextrin.

109. The solution of claim 103 or 104, wherein the solution comprises 15.5% (w/v) of the cyclodextrin.

110. The solution of any one of claims 103-109, wherein the buffer comprises boric acid.

111. The solution of any one of claims 103-110, wherein the preservative comprises a benzalkonium salt.

112. A method of treating a disorder mediated by an αv integrin, comprising topically administering to an eye of a subject in need thereof a therapeutically effective amount of a solution any one of claims 1-111 to treat the disorder.

113. The method of claim 112, wherein the αv integrin is an αvβ3 or αvβ5 integrin.

114. The method of claim 112, wherein the disorder is macular degeneration, diabetic retinopathy, macular edema, diabetic macular edema, or macular edema following retinal vein occlusion.

115. The method of claim 112, wherein the disorder is diabetic retinopathy.

116. The method of any one of claims 112-115, wherein the subject is an adult human.

117. The compound (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid hydrate.

118. The compound of claim 117, wherein the compound is (S)-3-(6-(difluoromethoxy)pyridin-3-yl)-3-(2-oxo-3-(3-(5,6,7,8-tetrahydro-1,8-naphthyridin-2-yl)propyl)imidazolidin-1-yl)propanoic acid monohydrate.

119. The compound of claim 117 or 118, wherein the compound is in crystalline form.

120. A method of preparing an aqueous, ophthalmic solution, the method comprising:
a. providing a first mixture comprising water, a cyclodextrin, and a compound of Formula I: 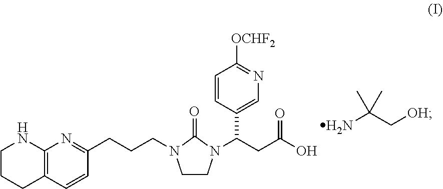
b. admixing a preservative and the first mixture, to thereby provide the aqueous, ophthalmic solution.

121. The method of claim 120, wherein the aqueous, ophthalmic solution has a pH in the range of 7.5 to 8.7.

122. The method of claim 120 or 121, wherein the preservative is benzalkonium halide.

123. The method of claim 120 or 121, wherein the preservative is benzalkonium chloride.

124. The method of claim 120 or 121, wherein the first mixture further comprises a buffer.

125. The method of claim 124, wherein the buffer comprises an organic acid.

126. The method of claim 124, wherein the buffer comprises boric acid.

127. The method of any one of claims 120-126, wherein solution comprises at least 75% w/w water.
